(19) United States
(12) Patent Application Publication
(10) Pub. No.: US 2007/0142635 A1
(43) Pub. Date: Jun. 21, 2007

(54) Process for preparing hexahydropyrimido[1,2-a]azepine-2-carboxylates and related compounds

(76) Inventors: David Askin, Warren, NJ (US); David Conlon, Plainsboro, NJ (US); Yoshinori Kohmura, Okazaki (JP); Jaemoon Lee, Edison, NJ (US); Brenda Pipik, Edison, NJ (US); Yong-Li Zhong, Edison, NJ (US)

Correspondence Address: MERCK AND CO., INC, P O BOX 2000, RAHWAY, NJ, 07065-0907, US

(21) Appl. No.: 10/582,414

(22) PCT Filed: Dec. 8, 2004
(86) PCT No.: PCT/US04/41115

§ 371 Date: Jun. 8, 2006

Related U.S. Application Data
(60) Provisional application No. 60/528,704, filed on Dec. 12, 2003.

(51) Int. Cl. C07F 9/6512; C07D 487/04

(52) U.S. Cl. 544/244; 544/281

(57) ABSTRACT

Processes for preparing 10-amino-3-hydroxy-4-oxo-4,6,7,8,9,10-hexahydropyrimido[1,2-a]azepine-2-carboxylates and related compounds are disclosed. The preparation of carboxamide derivatives from these carboxylates is also disclosed. The carboxamides are HIV integrase inhibitors and are useful for treating HIV infection and AIDS.

### FIELD OF THE INVENTION

The present invention is directed to processes for preparing 10-amino-3-hydroxy4-oxo-4,6,7,8,9,10-hexahydropyrimido[1,2-a]azepine-2-carboxylates and related compounds and to a class of substituted hydroxypyrimidinone carboxylates that can be employed as reactants in these processes. The hexahydropyriniidoazepine carboxylates and related compounds are useful as intermediates in the preparation of pharmacologically active compounds.

### BACKGROUND OF THE INVENTION

A class of hexahydropyrimido[1,2-a]azepine-2-carboxamides and related compounds are inhibitors of the HIV integrase enzyme. The compounds of Formulas XII, XIII and XIV as defined and described below are representative of this class. These compounds and pharmaceutically acceptable salts thereof are useful for preventing or treating infection by HIV and for treating or delaying the onset of AIDS. One approach to making these compounds is to prepare the oxime of a protected aminoazacycloalkanone (e.g., a Boc-protected aminoazepanone oxime), then conduct a Michael addition with the oxime using a suitable dialkylacetylene dicarboxylate and heat the resulting butenedioate product to cyclize the pyrimidine ring, and obtain thereby a carboxylate precursor which can then be converted to the desired carboxamide. The following Scheme A for preparing a hexahydropyrimido[1,2-α]azepine carboxamide illustrates this approach, wherein the Boc protecting group in P5 is subsequently removed (e.g., by treatment with acid) to give the desired carboxamide, whose unprotected amino group can optionally be derivatized by treatment with acylating agents, alkylating agents, and the like. 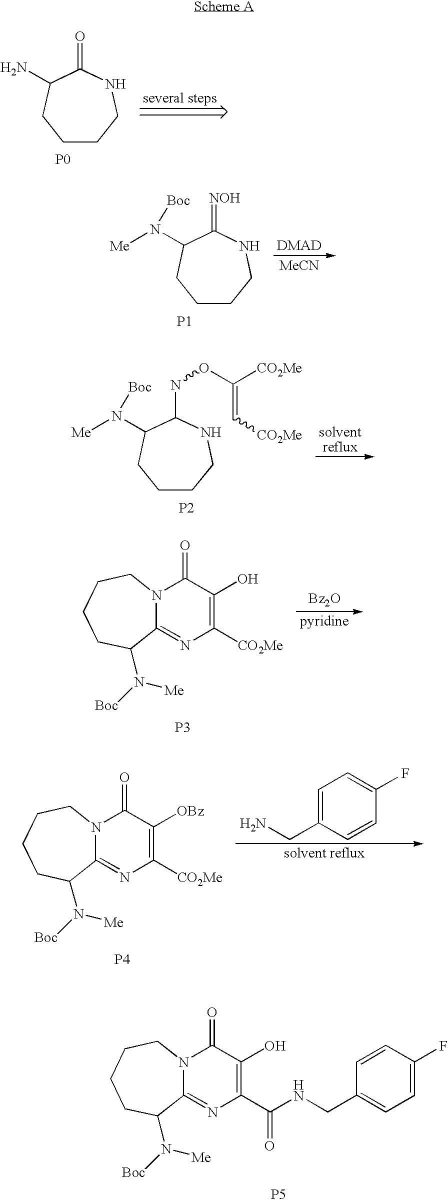
Unfortunately, the cyclization of the pyrimidine ring can be accompanied by the formation of significant by-product due to a competing second Michael addition; e.g., in Scheme A, the yield of P3 can be significantly and adversely affected by the formation of by-product P3′: 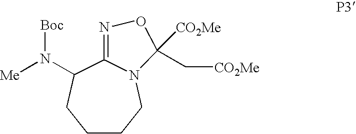
Furthermore, the preparation of the oxime (e.g., P1 in Scheme A) from the starting aminoazacycloalkanone (e.g., P0 in Scheme A) typically requires several steps which can have a low overall yield, and the starting aminoazacyclolalkanone is typically either expensive or unavailable commercially, in which case its synthesis from readily available starting materials is required, further reducing the overall yield. Accordingly, there is a need for an alternative less costly and/or higher yielding synthesis of the hexahydropyrimido[1,2-a]azepine-2-carboxylate intermediates and the corresponding carboxamide derivatives.

### SUMMARY OF THE INVENTION

The present invention is directed to processes for preparing 10-anino-3-hydroxy-4-oxo-4,6,7,8,9,10-hexahydropyrimido[1,2-a]azepine-2-carboxylates and related compounds and to processes for preparing carboxamide derivatives thereof. More particularly, the present invention includes a process for preparing a compound of Formula X or Formula XI: 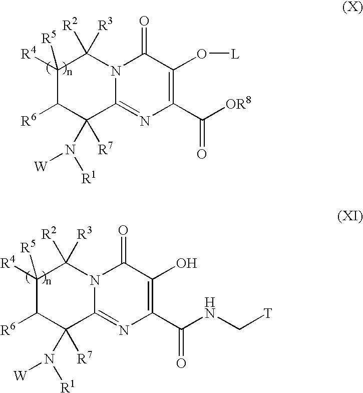
which comprises:

(H) contacting a compound of Formula VIII: 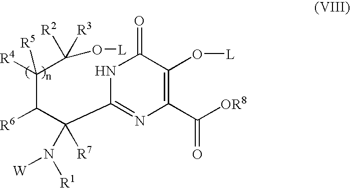
or a compound of Formula IX: 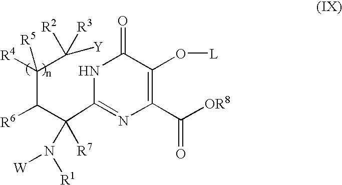
with a strong base to obtain Compound X; or

(H-1) contacting a compound of Formula VIII-1: 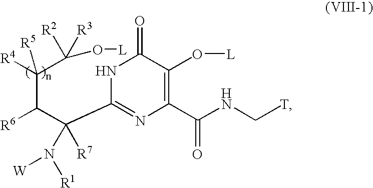
a compound of Formula VIII-2: 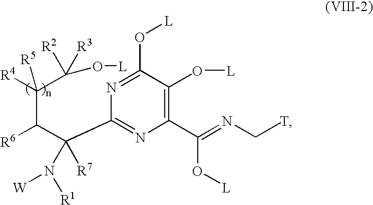
a compound of Formula VIII-3: 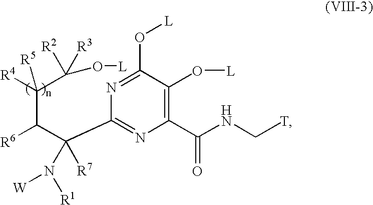
or a compound of Formula IX-1: 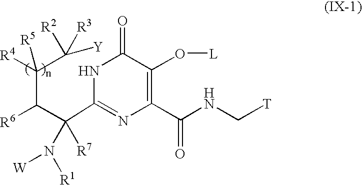
with a strong base to obtain Compound XI; wherein:
W is an amine protective group;
L is a hydroxy activating group;
Y is halo;
R¹ is:

(1) H,

(2) C₁₋₆ alkyl,

(3) C₁₋₆ alkyl substituted with O—C₁₋₆ alkyl, C₃₋₈ cycloalkyl, or aryl, wherein the cycloalkyl is optionally substituted with from 1 to 3 C₁₋₆ alkyl groups and the aryl is optionally substituted with from 1 to 5 substituents each of which is independently C₁₋₆ alkyl, O—C₁₋₆ alkyl, CF₃, OCF₃, halo, CN, or NO₂, or

(4) aryl which is optionally substituted with from 1 to 5 substituents each of which is independently C₁₋₆ alkyl, O—C₁₋₆ alkyl, CF₃, OCF₃, halo, CN, or NO₂;
R², R³, each R⁴, each R⁵, R⁶, and R⁷ are independently:

(1) H,

(2) C₁₋₆ alkyl, or

(3) C₁₋₆ alkyl substituted with O—C₁₋₆ alkyl, C₃₋₈ cycloalkyl, or aryl, wherein the cycloalkyl is optionally substituted with from 1 to 3 C₁₋₆ alkyl groups and the aryl is optionally substituted with from 1 to 5 substituents each of which is independently C₁₋₆ alkyl, O—C₁₋₆ alkyl, CF₃, OCF₃, halo, CN, or NO₂;
R⁸ is (i) a mixture of R^{A} and R^{B}, wherein R^{A} and R^{B} are different C₁₋₆ alkyl groups, or is (ii) R^{C}, wherein R^{C} is a C₁₋₆ alkyl;
each aryl is independently phenyl or naphthyl;
n is an integer equal to zero, 1, 2 or 3;
T is 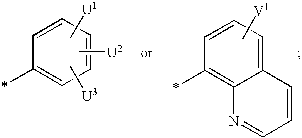
U¹, U² and U³ are each independently selected from the group consisting of H, halo, C₁₋₆ alkyl, O—C₁₋₆ alkyl, C₁₋₆ fluoroalkyl, SO₂-C₁₋₆ alkyl, C(═O)—NH(—C₁₋₆ alkyl), C(═O)—N(—C₁₋₆ alkyl)₂, and HetA;
V¹ is H, halo, C₁₋₆ alkyl, or C₁₋₆ fluoroalkyl; and
each HetA is independently a 5- or 6-membered heteroaromatic ring containing from 1 to 4 heteroatoms independently selected from N, O and S, wherein the heteroaromatic ring is optionally substituted with 1 or 2 C₁₋₆ alkyl groups.

The processes of the present invention can provide the bicyclic carboxylates of Formula X and bicyclic carboxamides of Formula XI in a significantly higher yield than the cyclization process described in the Background, which process is illustrated by the formation of P3 or P5 from P2 in Scheme A. Furthermore, the compounds of Formula VIII, IX, VIII-1, VIII-2, VIII-3 and IX-1 employed as reactants in the process of the invention can be prepared in relatively high yield from unsaturated cyclic ethers which themselves are either commercially available at a relatively cheap cost or which can be prepared in relatively high yield. Accordingly, the overall yield of Compound X or XI and derivatives thereof can be substantially higher than that of the process described in the Background. The advantages of the present invention are illustrated by a comparison of Scheme A in the Background with the following Scheme B representing an embodiment of the present invention: 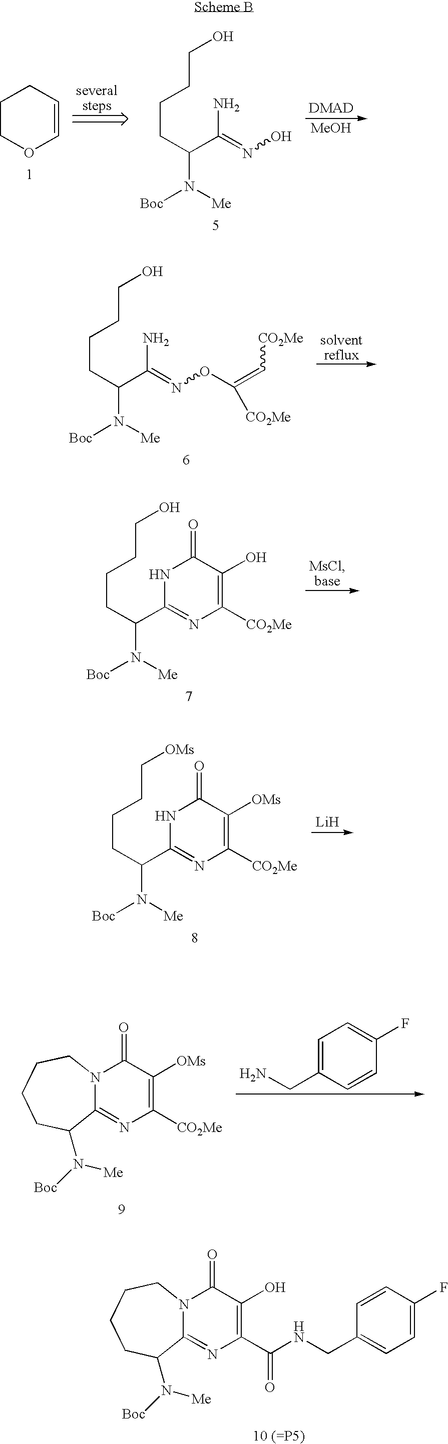
The cyclization in Scheme B (i.e., the formation of 9 from 8) has a higher yield than the corresponding cyclization in Scheme A (i.e., P3 from P2), at least in part because the Scheme B cyclization has no by-product due to a second Michael addition. The overall process of Scheme B (i.e., 1 to 9 or 10) has a significantly higher yield than that of Scheme A (P0 to P4 or P5). In addition, in contrast to P0 in Scheme A, the dihydropyran starting material 1 in Scheme B is a relatively cheap commodity chemical.

The present invention also provides an alternative one-pot synthesis for formation of 10 from 7 (1. amidation; 2. mesylation; and 3. cyclization) as outlined in the following Scheme C, where the amount of MsCl in the mesylation step does not need to be controlled to avoid mesylation of all hydroxyl groups: 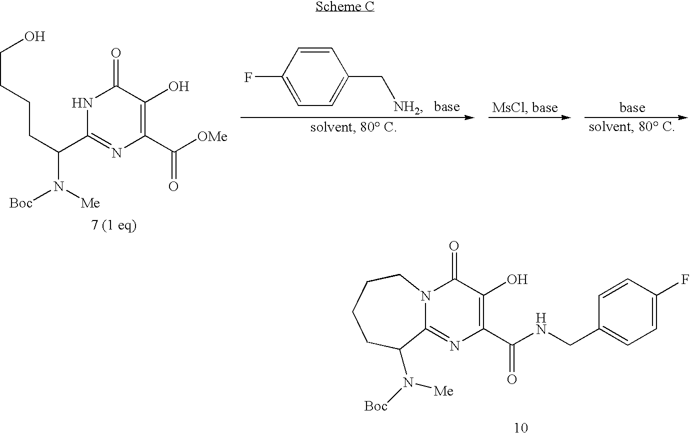
In the process outlined in Scheme B, when all hydroxyl groups are mesylated, the phenolic anion cannot be generated by anhydrous basic conditions (Scheme D). When the cyclization step is carried out under aqueous basic conditions, methyl ester is also hydrolyzed to give acid which is difficult to extract from aqueous layer. Hydrolysis does not occur if the amidation step is carried out before the cyclization step.

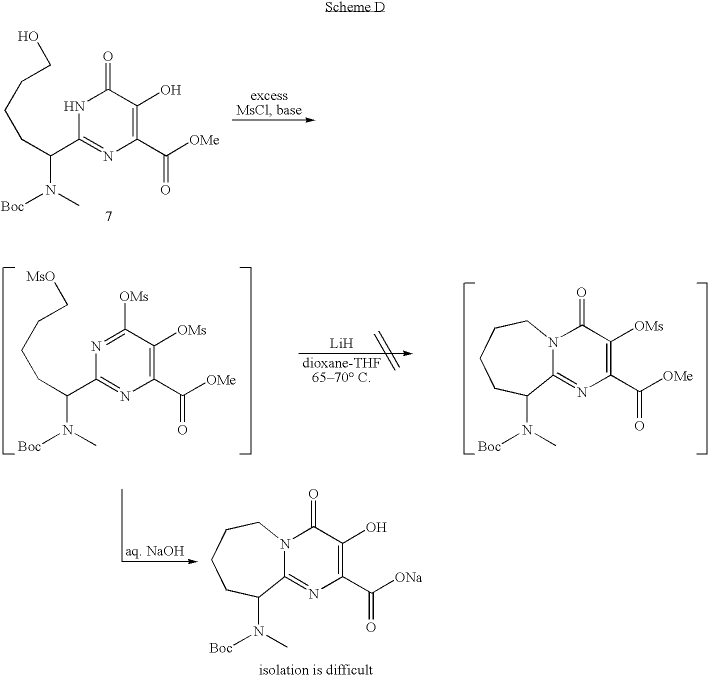

The present invention also includes a class of substituted hydroxypyrimidinone carboxylates and carboxamides that can be employed as reactants in the process set forth above. Additional classes of compounds encompassed by this invention are described below.

Various embodiments, aspects and features of the present invention are either described in or will be apparent from the ensuing description, example, and appended claims.

### DETAILED DESCRIPTION OF THE INVENTION

The present invention includes the processes set forth above in the Summary of the Invention, in which a compound of Formula X is prepared from either a compound of Formula VIII or a compound of Formula IX, or a compound of Formula XI is prepared from a compound of Formula VIII-1, a compound of Formula VIII-2, a compound of Formula VIII-3 or a compound of Formula IX-1. A compound of Formula X is alternatively referred to herein more simply as “Compound X”. Similarly, compounds of Formula VIII and IX are alternatively and respectively referred to as “Compound VIII” and “Compound IX”. Analogous nomenclature is employed for other compounds described herein.

Compounds VIII, IX, and X and compounds VIII-1, VIII-2, VIII-3 and IX-1 each contain one or more L groups, wherein L is a hydroxy activating group which, as described below, can be formed by treatment of the corresponding OH-containing precursors with a hydroxy activating agent. As used herein, the term “hydroxy activating agent” is a chemical reagent (e.g., a sulfonyl halide, a phosphinyl halide, etc.) that will form a derivatized hydroxy group (e.g., sulfonate, phosphinate, etc.) that is either (i) more reactive than hydroxy per se or (ii) confers reactivity where hydroxy per se is not reactive in the cyclization reaction in Step H or Step H-1. Correspondingly, a “hydroxy activating group” is a derivatized hydroxy group that provides either reactivity or improved reactivity with respect to the hydroxy group per se in Step H or Step H-1. While not wishing to be bound by any particular theory, the cyclization in Step H is believed to occur by nucleophilic attack of the deprotonated pyrimidinyl nitrogen on the aliphatic carbon substituted with the derivative OH group, wherein the derivatized hydroxy group is a better leaving group in nucleophilic substitution than hydroxy per se.

Compounds VIII, IX, and X and compounds VIII-1, VIII-2, VIII-3 and IX-1 also contain a group W, which is an amine protective group. The amine protective group W in these compounds can be any amine protective group that is stable with respect to the cyclization conditions employed in Step H or Step H-1 and any subsequent processing to a desired derivative (e.g., the coupling of Compound X with an amine in Step I to give a carboxarnide of Formula XI, as described below) and labile enough to be removed (cleaved) either from Compound X directly or from a subsequent derivative (e.g., the carboxamide of Formula XI) via contact with a suitable amine deprotecting agent to give the free amine with little or no degradation of any other functional groups present in the compound. Amine protective groups are known in the art and are described, for example, in Protective Groups in Organic Chemistry, edited by J. F. W. McOmie, Plenum Press, New York, 1973, pp. 43-74; and in T. W. Greene and P. G. M. Wuts, Protective Groups in Organic Synthesis, 2^{nd} edition, John Wiley, New York, 1991, pp. 309-385, the disclosures of which are herein incorporated by reference. Furthermore, the amine protective group W is typically also stable with respect to the reaction conditions encountered in Steps C to G described below for the preparation of precursors of Compound X or XI (i.e., “pre-steps” with respect to Step H or Step H-1), and accordingly the description below of the pre-steps refers only to group W. In the event a pre-step requires a different amine protective group W′, the overall process for preparing Compound X or XI incorporating the pre-step would additionally include protecting and deprotecting steps to add and later remove W′, with a subsequent protecting step to incorporate W prior to Step H or Step H-1. Further description of suitable amine protective groups for Step H or Step H-1 follows just below, and description of the formation and removal of such groups is provided further below, for example, in the descriptions of Step B and Step J.

An embodiment of the process of the invention is the process as set forth above wherein L is a sulfonate or a phosphinate; and all other variables are as originally defined (i.e., as defined in the Summary of the Invention).

Another embodiment of the process of the invention is the process as originally described above, wherein L is hydrocarbylsulfonyl, dihydrocarbylphosphinyl, or dihydrocarbyloxyphosphinyl; and all other variables are as originally defined.

Another embodiment of the process of the invention is the process as originally described, wherein L is:

(1) SO₂R^{I},

(2) P(O)(R^{J})₂, or

(3) P(O)(OR^{K})₂;
wherein
   R^{I} is (i) C₁₋₆ alkyl, (ii) C₁₋₆ haloalkyl, (iii) C₁₋₆ alkyl substituted with aryl, (iv) aryl, or (v) camphoryl;
   each R^{J} is independently (i) C₁₋₆ alkyl, (ii) C₁₋₆ haloalkyl, (iii) C₁₋₆ alkyl substituted with aryl, or (iv) aryl; and
   each R^{K} is independently (i) C₁₋₆ alkyl or (ii) C₁₋₆ alkyl substituted with aryl; and
   wherein any aryl defined in R^{I}, R^{J}, and R^{K} is optionally substituted with from 1 to 5 substituents each of which is independently halogen, —C₁₋₄ alkyl, —O—C₁₋₄ alkyl, CF₃, OCF₃, CN, or nitro;
      and all other variables are as originally defined.

Another embodiment of the process of the invention is the process as originally described, wherein L is SO₂R^{I}, wherein R^{I} is C₁₋₃ alkyl, CF₃, CF₂CF₃, CH₂CF₃, CH₂-aryl, aryl, or 10-camphoryl; wherein the aryl is optionally substituted with from 1 to 3 substituents each of which is independently F, Cl, Br, —C₁₋₄ alkyl, —O—C₁₋₄ alkyl, CF₃, OCF₃, or nitro; and all other variables are as originally defined.

In an aspect of the preceding embodiment, L is p-toluenesulfonyl, benzenesulfonyl, methanesulfonyl, trifluoromethanesulfonyl, p-nitrobenzenesulfonyl, naphthalenesulfonyl, or 10-camphorsulfonyl. In another aspect of the preceding embodiment L is methanesulfonyl.

Another embodiment of the process of the invention is the process as originally described, wherein the group formed by the 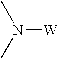
moiety in Compound X is a carbamate, an amide, or a tertiary amine; and all other variables are as originally defined or as defined in any one of the preceding embodiments. The term “carbamate” here refers to a group of formula 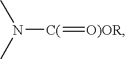
the term “amide” refers to a group of formula 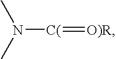
and the term “tertiary amine” refers to 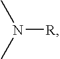
wherein in each case R independently represents an organic group which is chemically stable under reaction conditions employed in Step H and which can subsequently be cleaved selectively to afford the unprotected amine. Description of suitable R groups is provided below.

Another embodiment of the process of the invention is the process as originally described, wherein W is an amine protective group selected from the group consisting of:
(1) C₁₋₆ alkyl substituted with aryl, where the aryl is optionally substituted with from 1 to 5 substituents each of which is independently halo, —NO₂, —C₁₋₄ alkyl, or —O—C₁₋₄ alkyl,
(2) C(═O)—C₁₋₄ alkyl,
(3) C(═O)—C₁₋₄ haloalkyl,
(4) C(═O)—C₁₋₄ alkylene-aryl, where the aryl is optionally substituted with from 1 to 5 substituents each of which is independently halo, —NO₂, —C₁₋₄ alkyl, or —O—C₁₋₄ alkyl,
(5) C(═O)—O—C₁₋₄ alkyl,
(6) C(═O)—O—(CH₂)₀₋₁—CH═CH₂, and
(7) C(═O)—O—C₁₋₄ alkylene-aryl, where the aryl is optionally substituted with from 1 to 5 substituents each of which is independently halo, —NO₂, —C₁₋₄ alkyl, or —O—C₁₋₄ alkyl;
   and all other variables are as originally defined or as defined in any of the foregoing embodiments.

Still another embodiment of the process of the invention is the process as originally described, wherein W is an amine protective group selected from the group consisting of:
(1) —CH₂-phenyl, where the phenyl is optionally substituted with from 1 to 3 substituents each of which is independently halo, —NO₂, —C₁₋₄ alkyl, or —O—C₁₋₄ alkyl,
(2) —C(═O)—C₁₋₄ alkyl,
(3) —C(═O)—CF₃,
(4) —C(═O)—CCl₃,
(5) —C(═O)—CH₂-phenyl, where the phenyl is optionally substituted with from 1 to 3 substituents each of which is independently halo, —NO₂, —C₁₋₄ alkyl, or —O—C₁₋₄ alkyl,
(6) —C(═O)—O—C₁₋₄ alkyl,
(7) —C(═O)—O—CH₂—CH═CH₂, and
(8) —C(═O)—O—CH₂-phenyl, where the phenyl is optionally substituted with from 1 to 3 substituents each of which is independently halo, —NO₂, —C₁₋₄ alkyl, or —O—C₁₋₄ alkyl;
   and all other variables are as originally defined or as defined in any of the foregoing embodiments.

In an aspect of the preceding embodiment, W is t-butyloxycarbonyl (i.e., Boc), benzyloxycarbonyl (Cbz), allyloxycarbonyl (Alloc), p-nitrobenzyloxycarbonyl, p-methoxybenzyloxycarbonyl, p-bromobenzyloxycarbonyl, p-chlorobenzyloxycarbonyl, or 2,4-dichlorobenzyloxycarbonyl. In another aspect of the preceding embodiment, W is Boc.

Another embodiment of the process of the invention is the process as originally described, wherein R², R³, each R⁴, each R⁵, R⁶, and R⁷ are independently H or C₁₋₄ alkyl; and all other variables are as originally defined or as defined in any of the foregoing embodiments.

Another embodiment of the process of the invention is the process as originally described, wherein R², R³, each R⁴, each R⁵, R⁶, and R⁷ are all H; and all other variables are as originally defined or as defined in any of the foregoing embodiments.

Another embodiment of the process of the invention is the process as originally described, wherein R⁸ is R^{C} and R^{C} is a C₁₋₄ alkyl; and all other variables are as originally defined or as defined in any of the foregoing embodiments. In an aspect of the preceding embodiment, R⁸ is R^{C} and R^{C} is methyl.

Another embodiment of the process of the invention is the process as originally described, wherein n is an integer equal to 1 or 2; and all other variables are as originally defined or as defined in any of the foregoing embodiments. In an aspect of this embodiment, n is 1. In another aspect, n is 2.

Another embodiment of the process of the invention is the process as originally described, wherein T is 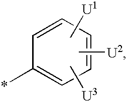
wherein U¹, U² and U³ are each independently H, halo, C₁₋₆ alkyl or C₁₋₆ fluoroalkyl; and all other variables are as originally defined or as defined in any of the foregoing embodiments. In an aspect of this embodiment, U¹, U² and U³ are each independently H or halo.

It is understood that the definition of any one of L, W, Y, R¹, R², R³, R⁴, R⁵, R⁶, R⁷, R⁸, R^{A}, R^{B}, R^{C}, R^{I}, R^{J}, R^{K}, T and n as originally set forth or as defined in any of the foregoing embodiments of the process, or aspects thereof, can be combined with the definition of any one or more of the others of L, W, Y, R¹, R², R³, R⁴, R⁵, R⁶, R⁷, R⁸, R^{A}, R^{B}, R^{C}, R^{I}, R^{J}, R^{K}, T and n as originally set forth or as defined in one of the foregoing embodiments or aspects thereof. Each such possible combination not expressly described above can be incorporated into the process of the invention, and each represents an additional embodiment of the process of the present invention.

Step H can be conducted in a solvent H. Step H-1 can be conducted in a solvent H-1. Suitable solvents for use as solvent H in Step H or solvent H-1 in Step H-1 include those selected from the group consisting of halogenated alkanes, alcohols, ethers, esters, tertiary amines, tertiary amides, N-alkylpyrrolidones, pyridines, sulfoxides, and nitriles. A class of solvents suitable for use as solvent H in Step H or solvent H-1 in Step H-1 consists of the solvents selected from the group consisting of C₁₋₁₀ linear and branched halogenated alkanes, C₁₋₆ alkyl alcohols, C₅₋₇ cycloalkyl alcohols, dialkyl ethers wherein each alkyl is independently a C₁₋₆ alkyl, C₁₋₆ linear and branched alkanes substituted with two —O—C₁₋₆ alkyl groups (which are the same or different), C₄-C₈ cyclic ethers and diethers, phenyl C₁₋₄ alkyl ethers, diethylene glycol di(C₁₋₄ alkyl) ethers, C₁₋₆ alkyl esters of C₁₋₆ alkylcarboxylic acids, tri-(C₁₋₆ alkyl)amnines, N,N-di-(C₁₋₆ alkyl)-C₁₋₆ alkylamides, N-(C₁₋₆ alkyl)pyrrolidones, pyridine, (mono- and di- and tri-C₁₋₆ alkyl)pyridines, di-(C₁₋₆ alkyl)sulfoxides, and C₂-C₆ aliphatic nitriles.

Representative examples of solvents suitable for use in Step H or Step H-1 include carbon tetrachloride, chloroform, methylene chloride, 1,2-dichloroethane, 1,1,2-trichloroethane, 1,1,2,2-tetrachloroethane, methanol, ethanol, isopropanol, n-butanol, t-butyl alcohol, cyclohexanol, cyclopentanol, ethyl ether, MTBE, THF, dioxane, 1,2-dimethoxyethane, anisole, phenetole, diglyme, methyl acetate, ethyl acetate, isopropyl acetate, triethylamine, tri-n-propylamine, diethylisopropylamine, diisopropylethylamine, DMF, DMAC, N-methylpyrrolidone, N-ethylpyrrolidone, pyridine, 2- or 3- or 4- picoline, 2,4,6-collidine, DMSO, acetonitrile, and propionitrile.

The contacting in Step H or Step H-1 is conducted in the presence of a strong base. While not wishing to be bound by any particular theory, it is believed that the base deprotonates the pyrimidinyl nitrogen so as to permit nucleophilic attack at the carbon bearing the aliphatic OH group which results in formation of the ring. Suitable bases include those selected from the group consisting of the alkali metals, alkali metal and alkaline earth metal halides, Group 2b transition metal halides, alkali metal salts and alkaline earth metal salts of di-C₁-C₆ alkylamines and C4-C8 cyclic secondary amiines, alkali metal salts and alkaline earth metal salts of bis(tri-C₁₋₄ alkylsilyl)amines, alkali metal and alkaline earth metal hydrides, C₁₋₆ alkyllithiums, aryllithiums, mono- and di-(C₁₋₆ alkyl)aryllithiums, C₁₋₆ alkylmagnesium halides, arylmagnesium halides, alkali metal amides, C₁₋₆ alkoxides of alkali and alkaline earth metals, alkali metal carbonates and bicarbonates, alkali metal phosphates, and alkali metal and alkaline earth metal hydroxides.

A class of suitable bases for use in Step H or Step H-1 consists of bases selected from the group consisting of alkali metal hydrides, alkaline earth metal hydrides, alkali metal amides, alkali metal C₁₋₆ alkoxides, alkaline earth metal di-C₁₋₆ alkoxides, alkali metal salts of bis(tri-C₁₋₄ alkylsilyl)amines, alkaline earth metal salts of bis(tri-C₁₋₄ alkylsilyl)amines, alkali metal carbonates, alkali metal bicarbonates, alkali metal and alkaline earth metal hydroxides. A sub-class of bases particularly suitable for use in Step H consists of the alkali metal hydrides and the alkaline earth metal hydrides (e.g., LiH, NaH, KH, MgH₂, and CaH₂). A sub-class of bases particularly suitable for use in Step H-1 consists of the alkali metal hydroxides and the alkaline earth metal hydroxides (e.g., LiOH, NaOH, KOH, Mg(OH)₂, and Ca(OH)₂).

Exemplary strong bases suitable for use in Step H or Step H-1 include lithium metal, methyllithium, n-butyllithium, tert-butyllithium, sec-butyllithium, phenyllithium, phenyl sodium, phenyl potassium, lithium amide, sodium amide, potassium amide, lithium tetramethylpiperidide, lithium diisopropylamide (LDA), lithium diethylamide, lithium dicyclohexylamide, sodium hexamethyldisilazide, lithium hexamethyldisilazide (LHDMS), sodium hydride, potassium hydride, magnesium hydride, lithium methoxide, sodium methoxide, potassium methoxide, lithium ethoxide, sodium ethoxide, potassium ethoxide, magnesium dimethoxide, magnesium dimethoxide, ethylmagnesium chloride, isopropylmagnesium chloride, phenylmagnesium chloride, ethylmagnesium bromide, isopropylmagnesium bromide, phenylmagnesium bromide, Na₂CO₃, K₂CO₃, Cs₂CO₃, KHCO₃, K₃PO₄, Na₃PO₄, Cs₃PO₄, LiOH, NaOH, KOH, Mg(OH)₂, and Ca(OH)₂.

The strong base can be employed in Step H in any proportion with respect to Compound VIII (or Compound IX) which will result in the formation of at least some of Compound X but it is typically employed in an amount that can optimize conversion of Compound VIII (or IX) and formation of Compound X. The strong base can be suitably employed in Step H in an amount of at least about 0.5 equivalent (e.g., from about 0.5 to 50 equivalents) per equivalent of Compound VIII. In one embodiment, the base is employed in an amount in a range of from about 0.8 to about 50 equivalents per equivalent of Compound VIII. The base is typically employed in an amount of at least about 1 equivalent (e.g., from about 1 to about 10 equivalents) per equivalent of Compound VIII, and is more typically employed in an amount in a range of from about 1.05 to about 2 equivalents (e.g., from about 1.2 to about 2 equivalents) per equivalent of Compound VIII.

The strong base can be employed in Step H-1 in any proportion with respect to Compound VIII-1, Compound VIII-2 and/or Compound VIII-3 (and/or IX-1) which will result in the formation of at least some of Compound XI, but it is typically employed in an amount that can optimize conversion of Compound VIII-1, VIII-2 and/or VIII-3 (and/or IX-1) and formation of Compound XI. The strong base can be suitably employed in Step H in an amount of at least about 0.5 equivalent (e.g., from about 0.5 to 50 equivalents) per equivalent of Compound VIII-1 and/or VIII-2 and/or VIII-3. In one embodiment, the base is employed in an amount in a range of from about 0.8 to about 50 equivalents per equivalent of Compound VIII-1 and/or VIII-2 and/or VIII-3. The base is typically employed in an amount of at least about 1 equivalent (e.g., from about 1 to about 10 equivalents) per equivalent of Compound VIII-1 and/or VIII-2 and/or VIII-3, and is more typically employed in an amount in a range of from about 4 to about 8 equivalents (e.g., from about 5 to about 8 equivalents) per equivalent of Compound VIII-1 and/or VIII-2 and/or VIII-3.

The contacting in Step H of Compound VIII or IX with the strong base can be conducted at any temperature at which the reaction (cyclization) forming Compound X can be detected. The reaction can suitably be conducted at a temperature in a range of from about −50 to about 200° C., and is typically conducted at a temperature in a range of from about −50 to about 120° C. In one embodiment, the temperature is in a range of from about −30 to about 100° C. (e.g., from about zero to about 80° C. or from about 25 to about 80° C.).

The contacting in Step H-1 of Compound VIII-1, VIII-2, VIII-3 or IX-1 with the strong base can be conducted at any temperature at which the reaction (cyclization) forming Compound XI can be detected. The reaction can suitably be conducted at a temperature in a range of from about −50 to about 200° C., and is typically conducted at a temperature in a range of from about −50 to about 120° C. In one embodiment, the temperature is in a range of from about −30 to about 100° C. (e.g., from about zero to about 90° C. or from about 25 to about 90° C.).

In a particularly suitable embodiment of Step H, the contacting is conducted in an ether solvent (e.g., THF or dioxane), the strong base is an alkali metal hydride (e.g., LiH, NaH, or KH), the temperature is in a range of from about 0 to about 80° C. (e.g., from about 25 to about 80° C.), and the base is employed in an amount of at least about 1 equivalent (e.g., from about 1.05 to about 2 equivalents) per equivalent of Compound VIII.

In a particularly suitable embodiment of Step H-1, the contacting is conducted in an aqueous environment (e.g., DMAC-H₂O), the strong base is an alkali metal hydroxide (e.g., LiOH, NaOH, or KOH), the temperature is in a range of from about 0 to about 100° C. (e.g., from about 25 to about 90° C.), and the base is employed in an amount of in a range of from about 4 to about 8 equivalents (e.g., from about 5 to about 8 equivalents) per equivalent of Compound VIII-1 and/or VIII-2 and/or VIII-3.

The reaction of Step H or Step H-1 can be conducted by forming a mixture (typically a solution) of Compound VIII (or IX) or Compound VIII-1 (VIII-2, VIII-3 or IX-1), respectively, in a suitable organic solvent at a temperature below the desired reaction temperature, charging the strong base thereto, and then bringing the resulting mixture to reaction temperature and maintaining the mixture at reaction temperature (optionally with agitation such as stirring) until the reaction is complete or the desired degree of conversion of the reactants is achieved. The reaction time can vary widely depending upon, inter alia, the reaction temperature and the choice and relative amounts of reactant and base, but the reaction time for complete conversion is typically in a range of from about 1 to about 24 hours (e.g., from about 2 to about 18 hours). Compound X or XI can subsequently be isolated (alternatively referred to as recovered) from the reaction mixture using conventional procedures, such as crystallization from a suitable organic solvent or chromatography.

The present invention includes a process for preparing a compound of Formula X which comprises Step H or preparing a compound of Formula XI which comprises Step H-1 as described above; and which further comprises:
(F1) treating a compound of Formula VIII: 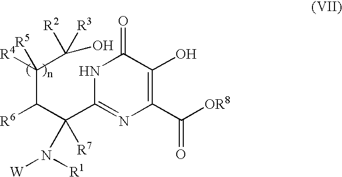
with a hydroxy activating agent to form a product which is (i) the compound of Formula VIII, (ii) a compound of Formula VIIIa: 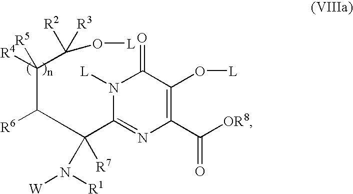
or (iii) a mixture of Compound VIII and Compound VIIIa;

(F2) then:

(1) when the product is (i) Compound VIII, proceeding directly to Step G or to Step H;

(2) when the product is (ii) Compound VIIIa, contacting the product with (a) a primary or secondary amine or (b) an alcohol, water, or an alcohol-water mixture in the presence of a base, to form Compound VIII; and

(3) when the product is (iii) a mixture of Compounds VIII and VIIIa, optionally contacting the product with (a) a primary or secondary amine or (b) an alcohol, water, or an alcohol-water mixture in the presence of a base, to form additional Compound VIII; and

(G) optionally reacting Compound VIII from Step F2 with a halide salt to form the compound of Formula IX.

The present invention also includes a process for preparing a compound of Formula XI which comprises Step H-1 as described above; and which further comprises:
(F1-1) reacting a compound of Formula VIII with an amine of formula T-CH₂NH₂ to obtain a compound of Formula VIII-1:

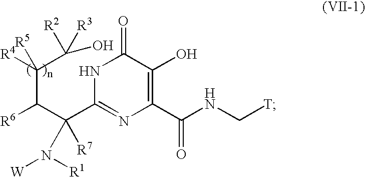

(F1-2) treating a compound of Formula VIII-1 with a hydroxy activating agent to form a product which is (i) a compound of Formula VIII-1, (ii) a compound of Formula VIII-2, (iii) a compound of Formula VIII-3, (iv) a compound of Formula VIII-1a, or (v) a mixture of two to four components selected from the group consisting of Compound VIII-1, Compound VIII-2, Compound VIII-3 and Compound VIII-1a;

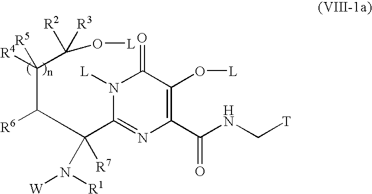

(F2-1) then:

(1) when the product is (i) a compound of Formula VIII-1, (ii) a compound of Formula VIII-2, (iii) a compound of Formula VIII-3, or a mixture thereof, proceeding directly to Step G-1 or to Step H-1;

(2) when the product is (iv) Compound VIII-1a, contacting the product with (a) a primary or secondary amine or (b) an alcohol, water, or an alcohol-water mixture in the presence of a base, to form Compound VIII-1; and

(3) when the product is the mixture (v) containing Compound VIII-1a, optionally contacting the product with (a) a primary or secondary amine or (b) an alcohol, water, or an alcohol-water mixture in the presence of a base, to form additional Compound VIII-1; and

(G-1) optionally reacting Compound VIII-1 from Step F2-1 with a halide salt to form a compound of Formula IX-1.

Suitable hydroxy activating agents for use in Step F1 or Step F1-2 include those selected from the group consisting of sulfonylating agents and phosphinating agents, wherein each of the resulting O-L groups in Compound VIII, VIII-1, VIII-2, or VIII-3 is respectively a sulfonate or a phosphinate. Treatment with a sulfonylating agent or a phosphinating agent is typically conducted in the presence of a base. A class of suitable activating agents includes agents of formula L-X, wherein L is hydrocarbylsulfonyl, dihydrocarbylphosphinyl, or dihydrocarbyloxyphosphinyl, and X is halogen. A sub-class of the preceding class of suitable activating agents includes agents of formula L-X, wherein L is R^{I}SO₂, (R^{J})₂P(O), or (R^{K}O)₂P(O); X is halogen; and R^{I}, each R^{J}, and each R^{K} are each as defined above in the description of Step H. Another sub-class of suitable agents includes agents of formula R^{I}SO₂X wherein X is halogen, and R^{I} is as defmed above in the description of Step H or Step H-1. Still another sub-class of suitable agents includes consists of p-toluenesulfonyl halides, benzenesulfonyl halides, methanesulfonyl halides, trifluoromethanesulfonyl halides, p-nitrobenzenesulfonyl halides, naphthalenesulfonyl halides, and 10-camphorsulfonyl halides.

Representative examples of suitable hydroxy activating agents of formula L-X are p-toluenesulfonyl chloride, benzenesulfonyl chloride, methanesulfonyl chloride, trifluoromethanesulfonyl chloride, p-nitrobenzenesulfonyl chloride, naphthalenesulfonyl chloride, 10-camphorsulfonyl chloride, methanesulfonyl bromide, and p-toluenesulfonyl bromide.

The treatment of Compound VIII in Step F1 or Compound VIII-1 in Step F1-2 can be conducted in a solvent F1 or F1-2 which is an aprotic solvent. Suitable solvents include those selected from the group consisting of alkanes, cycloalkanes, halogenated alkanes, halogenated cycloalkanes, aromatic hydrocarbons, alkylated aromatic hydrocarbons, halogenated aromatic hydrocarbons, alkylated and halogenated aromatic hydrocarbons, ethers, esters, tertiary amides, sulfoxides, and nitriles. A class of solvents suitable for use as solvent F1 in Step F1 or as solvent F1-2 in Step F1-2 consists of the solvents selected from the group consisting of C₁₋₁₀ linear and branched alkanes, C₁₋₁₀ linear and branched halogenated alkanes, C₅₋₁₀ cycloalkanes, halogenated C₅₋₁₀ cycloalkanes, benzene, naphthalene, mono- and di- and tri-C₁₋₆ alkyl substituted benzenes, halogenated benzenes, halogenated mono- and di- and tri-C₁₋₆ alkyl substituted benzenes, dialkyl ethers wherein each alkyl is independently a C₁₋₆ alkyl, C₁₋₆ linear and branched alkanes substituted with two —O—C₁₋₆ alkyl groups (which are the same or different), C₄-C₈ cyclic ethers and diethers, phenyl C₁₋₄ alkyl ethers, diethylene glycol di(C₁₋₄ alkyl) ethers, C₁₋₆ alkyl esters of C₁₋₆ alkylcarboxylic acids, N,N-di-(C₁₋₆ alkyl)-C₁₋₆ alkylamides, di-(C₁₋₆ alkyl)sulfoxides, and C₂-C₆ aliphatic nitriles.

Representative examples of solvents suitable for use in Step F1 or Step F1-2 include exemplary halogenated alkanes, ethers, esters, tertiary amides, sulfoxides and nitriles listed above in the discussion of solvents for Step H or Step H-1, and also include the following: pentane (individual isomers and mixtures thereof), hexane (individual isomers and mixtures thereof), heptane (individual isomers and mixtures thereof), cyclopentane, cyclohexane, cycloheptane, chlorocyclopentane, chlorocyclohexane, benzene, toluene, o- and m- and p-xylene, xylene mixtures, ethylbenzene, chlorobenzene, bromobenzene, o-chlorotoluene, 2,4-dichlorotoluene, and 2,4,6-trichlorotoluene.

The treatment in Step F1 or Step F1-2 can be conducted in the presence of a base, wherein the role of the base is to neutralize the acid by-product (e.g., HX such as HCl) caused by the derivatization (e.g., sulfonylation or phosphination with an L-X agent as described above) of the OH groups. Suitable bases included those selected from the group consisting of tertiary alkyl amines, tertiary cyclic amines, and diazabicycloalkenes. Representative examples of suitable bases include TEA, DIPEA, NMM, DBU, DBN, DABCO, tri-n-propylamine, tri-isopropylamine, or tri-n-butylamine.

In a particularly suitable embodiment, Step F1 is conducted in a solvent as described above and in the presence of a base as described above.

In another particularly suitable embodiment, Step F1-2 is conducted in a solvent as described above and in the presence of a base as described above.

The hydroxy activating agent can be employed in Step F1 in any proportion with respect to Compound VII which will result in the formation of at least some of Compound VIII and/or VIIIa, but it is typically employed in an amount that can optimize conversion to Compound VIII and/or VIIIa. The hydroxy activating agent is suitably employed in an amount of at least about 0.5 equivalent per equivalent of Compound VII, and is typically employed in an amount of at least about 1 equivalent (e.g., from about 1 to about 50 equivalents) per equivalent of Compound VII. The hydroxy activating agent is more typically employed in an amount in a range of from about 1.5 to about 5 equivalents (e.g., from about 2 to about 4 equivalents) per equivalent of Compound VII.

The hydroxy activating agent can be employed in Step F1-2 in any proportion with respect to Compound VII-1 which will result in the formation of at least some of Compound VIII-1, VIII-2, VIII-3 and/or VIII-1a, but it is typically employed in an amount that can optimize conversion to Compound VIII-1, VIII-2, VIII-3 and/or VIII-1a. The hydroxy activating agent is suitably employed in an amount of at least about 0.5 equivalent per equivalent of Compound VII-1, and is typically employed in an amount of at least about 1 equivalent (e.g., from about 1 to about 50 equivalents) per equivalent of Compound VII-1. The hydroxy activating agent is more typically employed in an amount in a range of from about 1.5 to about 8 equivalents (e.g., from about 4 to about 8 equivalents) per equivalent of Compound VII-1.

The treatment in Step F1 or Step F1-2 can be conducted at any temperature at which the reaction to form the desired products can be detected. The temperature is suitably in a range of from about 45 to about 200° C., and is typically in a range of from about −30 to about 100° C. (e.g., from about −15 to about 50° C.), and is more typically in a range of from about −5 to about 30° C.

When base is employed in Step F1 or Step F1-2, it is suitably employed in an amount of at least one equivalent per equivalent of hydroxy activating agent, is typically employed in an amount of from about 1 to about 2 equivalents per equivalent of hydroxy activating agent, and is more typically employed in a ratio of about 1 equivalent per equivalent of hydroxy activating agent.

When the product of Step F1 is Compound Via or is a mixture of Compound VIII and Compound VIIIa or the product of Step F1-2 is Compound VIII-1a or a mixture containing Compound VIII-1a, the product is or can be contacted in Step F2 or Step F2-1, respectively, with either (i) a primary or secondary amine or (ii) an alcohol, water, or an alcohol-water mixture (e.g., a mixture comprising from about 1 to about 99 vol. % water based on the total volume of alcohol and water) in the presence of base, in order to convert some or all of the Compound VIIIa to Compound VIII or Compound VIII-1a to Compound VIII-1 for use in optional Step G and in Step H. When an amine is employed, it is suitably a C₁₋₆ alkylamine or a di-C₁₋₆ alkylamine. When an alcohol, water, or an alcohol-water mixture is employed, it is suitable to use a C₁₋₆ alkyl alcohol (e.g., methanol, ethanol, or isopropanol) in the presence of an alkali metal carbonate, an alkali metal hydroxide, or an alkaline earth metal hydroxide.

The amine is suitably employed in Step F2 or Step F2-1 in an amount of at least about 0.5 equivalent per equivalent of Compound VII or VII-1, respectively, and is more typically employed in an amount in a range of from about 1 to about 10 equivalents per equivalent of Compound VII or VII-1, respectively.

When the alcohol-base combination is employed, the base is suitably employed in Step F2 or Step F2-1 in a catalytic amount or an amount in excess of a catalytic amount. Accordingly, the base can be employed in amount of in a range of from about 0.05 to about 10 equivalents per equivalent of Compound VII or VII-1. When water or an alcohol-water combination is employed, the base is suitably employed in an amount of at least about 0.5 equivalent per equivalent of Compound VII or VII-1, and is typically employed in an amount in a range of from about 1 to about 10 equivalents per equivalent of Compound VII or VII-1. Although at least about 0.5 equivalent of alcohol and/or water per equivalent of Compound VII or VII-1 is suitably employed in Step F2 or Step F2-1, and at least about 1 equivalent of alcohol and/water per equivalent of Compound VII or VII-1 is typically employed, the alcohol and/or water is more typically present in substantial excess and can be employed as the solvent.

The contacting in Step F2 or Step F2-1 can be conducted at any temperature at which the reaction to convert Compound VIIIa to Compound VIII or Compound VIII-1a to Compound VIII-1 can be detected. The temperature is suitably in a range of from about −50 to about 200° C., and is typically in a range of from about −10 to 40° C., and is more typically in a range of from about zero to about 30° C.

The treatment in Step F1 or Step F1-2 can be conducted by charging Compound VIII or VII-1 and a suitable solvent to a suitable reaction vessel, followed by the slow addition of the hydroxy activating agent and base (if employed), bringing the resulting mixture to reaction temperature, and maintaining the mixture at reaction temperature (optionally with agitation such as stirring) until the reaction is complete or the desired degree of conversion to Compounds VIII and/or VIIIa or to Compounds VIII-1, VIII-2, VIII-3 and/or VIII-1a is achieved. The reaction time can vary widely depending upon, inter alia, the reaction temperature and the choice and relative amounts of reactant, activating agent, and base, but the reaction time for complete conversion is typically in a range of from about 0.5 to about 24 hours (e.g., from about 1 to about 12 hours).

In Step F2 or Step F2-1, the primary/secondary amine or the alcohol (or water or water+alcohol)-base combination can be added directly to the reaction vessel containing the product which is Compound VIIIa or the mixture of Compounds VIII and VIIIa, or the product which is Compound VIII-1a or the mixture containing Compound VM-la, and the admixture maintained at reaction temperature until the desired degree of conversion of VIIIa to VIII or VIII-1a to VIII-1 is achieved. Alternatively, the Step F1 or Step F2-1 product can be isolated using conventional procedures such as chromatography or crystallization from solvent, and redissolved in a suitable solvent F2 or F2-1 (e.g., an ether, a nitrile, or an ester) or the product can be concentrated and solvent switched from a solvent F1 (or solvent F1-2) to a solvent F2 (solvent F2-1) without isolation, followed by addition of the amine or the alcohol (or water or water+alcohol)-base combination, and then aging of the mixture at a suitable temperature. In one embodiment, the Step F1 or Step F2-1 product is solvent switched to the alcohol of the alcohol-base combination, followed by addition of the base, and then aging of the mixture at a suitable reaction temperature. In each of the foregoing procedures, the aging time can vary widely depending upon, initer alia, the aging temperature and the choice and relative amounts of reagent, but the reaction time for complete conversion is typically in a range of from about 0.5 to about 100 hours (e.g., from about 1 to about 12 hours). After aging, the Compound VIII (or VIII-1) product from Step F2 (or Step F2-1, respectively), can be isolated using conventional procedures such as chromatography or solvent crystallization, or solvent switched for use in Step G (or G-1) and/or Step H (or H-1). Alternatively, the reaction mixture containing Compound VIII in solvent F2 or Compound VIII-1 in solvent F2-1, after suitable washing and other treatment to remove impurities and unreacted reactant or reagent, can be employed directly in optional Step G or optional Step G-1, or Step H or Step H-1.

Step F1-1 concerns with the coupling of Compound VII with an amine of formula T-CH₂NH₂ to obtain Compound VII-1. The coupling reaction is suitably conducted in solvent at a temperature in the range of from about 40 to about 200° C., and is typically conducted at a temperature in the range of from about 50 to about 160° C. In one embodiment, the coupling reaction is conducted at a temperature in the range of from about 70 to about 90° C. In another embodiment, the coupling reaction is conducted at solvent reflux at atmospheric pressure, wherein the solvent is chosen to provide the desired reflux temperature. Solvents suitable for use in Step F1-1 include those selected from the group consisting of alkanes, cycloalkanes, aromatic hydrocarbons, halogenated alkanes, halogenated cycloalkanes, alcohols, esters, ethers, nitrites and tertiary amides. Further description of these solvent classes is set forth above in the discussion of solvents suitable for use in Step F1, Step H-1, and other steps. These earlier descriptions are applicable here, and are herein incorporated. A class of solvents suitable for use in Step F1-1 consists of those selected from the group consisting of alcohols, esters, ethers and tertiary amides. A sub-class of this class consists of the solvents selected from the group consisting of C₁-C₆ alkyl alcohols, dialkyl ethers wherein each alkyl is independently a C₁-C4 alkyl, C₄-C₅ cyclic ethers, C₁-C4 alkyl esters of C₁-C₄ alkylcarboxylic acids, and C₁-C₄ alkyl amides of C₁-C₄ alkylcarboxylic acids. Another sub-class of this class is a solvent selected from the group consisting of methanol, ethanol, n-propanol, isopropanol, t-butyl alcohol, diethylether, 1,2-dimethoxyethane, THF, methyl acetate, ethyl acetate, isopropyl acetate and N,N′dimethylacetamide.

The amine of formula T-CH₂NH₂ can be employed in Step F1-1 in any proportion which will result in the formation of at least some of Compound VII-1. Typically, however, the reactants are employed in proportions which can optimize conversion of at least one of the reactants, and usually the amine is employed in an amount that can optimize the conversion of Compound VII. The amine can be suitably employed in an amount of at least about 0.5 equivalent (e.g., in a range of from about 0.5 to about 10 equivalents) per equivalent of Compound VII. It is preferred to use an excess of amine in order to increase the degree of conversion and/or shorten the reaction time. Accordingly, the amine is typically employed in an amount of at least about 1.05 equivalents per equivalent of Compound VII, and is more typically employed in an amount in a range of from about 1.1 to about 10 equivalents, or from about 1.1 to about 5 equivalents, or from about 1.1 to about 2 equivalents (e.g., about 1.1 to 1.7 equivalents), per equivalent of Compound VII.

Step F1-1 can be conducted in the presence or absence of a base. Suitable bases included those selected from the group consisting of tertiary alkyl amines, tertiary cyclic amines, and diazabicycloalkenes. Representative examples of suitable bases include TEA, DIPEA, NMM, DBU, DBN, DABCO, tri-n-propylamine, tri-isopropylamine, or tri-n-butylamime.

The reaction of Step F1-1 can be suitably conducted by adding the amine of formula T-CH₂NH₂ to a solution or suspension of Compound VII in the selected solvent and then heating the mixture to reaction temperature and maintaining at reaction temperature until the reaction is complete or the desired degree of conversion of the reactants is achieved. Isolation of the amide product VII-1 can be accomplished using conventional procedures, and the isolated product can be re-dissolved for use in Step F1-2. Alternatively the reaction mixture containing product VII-1 can be used directly in Step F1-2.

Amines of formula T-CH₂NH₂ can be prepared using the methods described in Richard Larock, Comprehensive Organic Transformations, VCH Publishers Inc, 1989, pp 385-438, or as described in Morrison and Boyd,Organic Chemistry, 4^{th} edition, Allyn and Bacon, 1983, pp. 893-897, or routine variations thereof.

Step G is an optional step in which Compound VIII resulting from Step F2 can be converted by reaction with a halide salt to the halide compound IX. Step G-1 is an optional step in which Compound VIII-1 resulting from Step F2-1 can be converted by reaction with a halide salt to the halide compound IX-1.

Suitable halide salts for use in Step G or Step G-1 include salts selected from the group consisting of alkali metal halide salts, alkaline earth metal halide salts, and quaternary ammonium halide salts. A class of suitable halide salts consists of salts selected from the group consisting of LiBr, LiCl, Lil, NaBr, NaCl, Nal, KBr, KCl, KI, MgBr₂, MgCl₂, and quaternary ammonium halide salts of formula (C₁₋₄ alkyl)₄N-halide in which the halide is chloride, bromide, or iodide.

Step G or Step G-1 can be conducted in a solvent G or G-1, respectively. Suitable solvents for Step G or G-1 include those selected from the group consisting of esters, nitriles, tertiary amides, sulfoxides, and ketones. The esters, nitrites, tertiary amides, and sulfoxides described above as suitable for use as solvent H in Step H are also suitable for use as solvents in Step G or Step G-1, and accordingly the earlier description of those solvent classes is incorporated herein by reference. Ketones, not heretofore described, are also suitable as solvents in Step G or Step G-1. More particularly, suitable ketones include di-C₂₋₁₀ alkanones and C₄₋₈ cycloalkanones. Representative examples of ketone solvents suitable for use in Step G include acetone, ethyl ketone, methyl ethyl ketone, methyl isoproypl ketone, methyl isobutyl ketone, 2-pentanone, cyclopentanone, and cyclohexanone.

The halide salt can be employed in Step G or Step G-1 in any proportion with respect to Compound VIII or VIII-1 which will result in the formation of at least some of Compound IX or IX-1, but it is typically employed in an amount that can optimize conversion to Compound IX or IX-1. The halide salt is suitably employed in an amount of at least about 0.5 equivalent per equivalent of Compound VIII or VIII-1, and is typically employed in an amount of at least about 1 equivalent (e.g., from about 1 to about 50 equivalents) per equivalent of Compound VIII or VIII-1. The halide salt is more typically employed in an amount in a range of from about 1 to about 10 equivalents (e.g., from about 2 to about 5 equivalents) per equivalent of Compound VIII or VIII-1.

The reaction of optional Step G or optional Step G-1 can be conducted at any temperature at which formation of Compound IX or IX-1 can be detected. The temperature is suitably in a range of from about −45 to about 200° C., and is typically in a range of from about −10 to about 100° C. (e.g., from about 20 to about 80° C.), and is more typically in a range of from about 40 to about 60° C.

The reaction of optional Step G or optional Step G-1 can be conducted by forming a mixture (typically a solution) of Compound VIII or VIII-1 in a suitable organic solvent at a temperature below the desired reaction temperature, charging the halide salt thereto, and then bringing the resulting mixture to reaction temperature and maintaining the mixture at reaction temperature (optionally with agitation such as stirring) until the reaction is complete or the desired degree of conversion of Compound VIII or VIII-1 is achieved. The reaction time can vary widely depending upon, iizter alia, the reaction temperature and the choice and relative amounts of reactant and base, but the reaction time for complete conversion is typically in a range of from about 1 to about 24 hours (e.g., from about 2 to about 12 hours). Compound IX or IX-1 can subsequently be isolated (alternatively referred to as recovered) from the reaction mixture using conventional procedures and then redissolved for use in Step H or Step H-1, or alternatively the reaction mixture containing Compound IX or IX-1 can be concentrated and solvent switched for use in Step H or Step H-1, respectively.

The present invention includes a process for preparing a compound of Formula X which comprises Steps F1, F2, G and H as described above; and which further comprises:

(E) heating (i) a mixture of compounds of Formula VIa and VIb or (ii) a compound of Formula VIc: 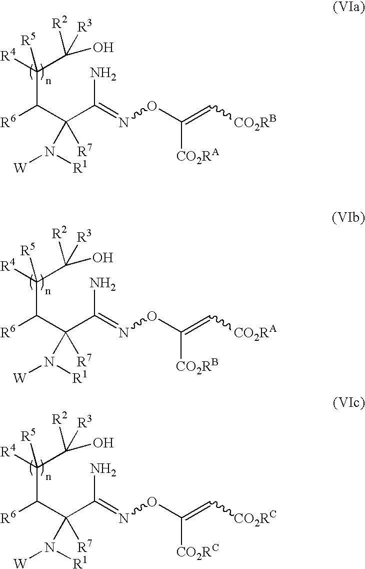
to obtain Compound VII.

The present invention includes a process for preparing a compound of Formula XI which comprises Steps F1-1, F1-2, F2-1, G-1 and H-1 as described above; and which further comprises Step E as described above.

Step E can be conducted in a solvent E. Suitable solvents include those selected from the group consisting of alcohols, esters, ethers, tertiary amides, nitriles, aromatic hydrocarbons, halogenated aromatic hydrocarbons, alkylated aromatic hydrocarbons, and halogenated and alkylated aromatic hydrocarbons. A class of solvents suitable for use as solvent E in Step E consists of the solvents selected from the group consisting of C₁₋₁₀ alkyl alcohols, C₅₋₁₀ cycloalkyl alcohols, C₁₋₆ alkyl esters of C₁₋₆ alkylcarboxylic acids, dialkyl ethers wherein each alkyl is independently a C₁₋₁₀ alkyl, C₁₋₁₀ linear and branched alkanes substituted with two —O—C₁₋₁₀ alkyl groups (which are the same or different), C₄₋₈ cyclic ethers and diethers, phenyl C₁₋₄ alkyl ethers, N,N-di-C₁₋₆ alkyl)-C₁₋₆ alkylamides, C₂₋₆ aliphatic nitriles, benzene, naphthalene, mono- and di- and tri-C₁₋₆ alkyl substituted benzenes, halogenated benzenes, halogenated mono- and di- and tri-C₁₋₆ alkyl substituted benzenes, and diethylene glycol di(C₁₋₄ alkyl) ethers.

Representative examples of solvents in the above solvent classes suitable for use in Step E are the same as those listed earlier in the description of solvents suitable as solvent H or H-1 in Step H or H-1 and/or suitable as solvent F1 in Step F1 or as solvent F1-2 in Step F1-2, and are herein incorporated by reference.

The reaction of Step E can be conducted at any temperature at which formation of Compound VII can be detected. The temperature is suitably at least about 80° C. (e.g., in a range of from about 80 to about 200° C.), and is typically at least about 90° C. (e.g., in a range of from about 100 to about 200° C.), and is more typically at least about 100° C. (e.g., in a range of from about 110 to about 160° C.). When a solvent is employed in Step E, the heating can be conducted under atmospheric pressure at the reflux temperature of the solvent. Alternatively, if a low-boiling solvent is employed, the heating can be conducted under pressure to achieve the desired temperature. It is typically preferred, however, to conduct Step E at atmospheric pressure.

It is particularly suitable to employ a solvent E in Step E which has a boiling point of at least about 90° C., and it is preferred to employ a solvent E in Step E which has a boiling point of at least about 110° C. A suitable class of solvents having a boiling point at or above 90° C. includes those selected from the group consisting of C4-10 alkyl alcohols, a C₅₋₁₀ cycloalkyl alcohols, C₃₋₆ alkyl esters of C₁₋₆ alkylcarboxylic acids, C₁₋₆ alkyl esters of C₃₋₆ alkylcarboxylic acids, phenyl C₁₋₄ alkyl ethers, C₃₋₆ aliphatic nitriles, C₇₋₁₀ alkylbenzenes, monohalobenzenes, dihalobenzenes, trihalobenzenes, (halo)-(C₁₋₄ alkyl)-benzenes, (dihalo)-(C₁₋₄ alkyl)-benzenes, (di-C₁₋₄ alkyl)-(halo)-benzenes, diethylene glycol di(C₁₋₄ alkyl) ethers, C₆₋₈ cyclic ethers, C₅₋₈ cyclic diethers, or (di-C₄₋₆ alkyl) ethers.

Representative examples of solvents suitable for use in Step E and having a boiling point of 90° C. or more include n-propanol, n-butanol, sec-butyl alcohol, n-decyl alcohol, n-octyl alcohol, cyclohexanol, cyclopentanol, cycloheptanol, anisole, phenetole, toluene, o-xylene, m-xylene, p-xylene, mesitylene, ethylbenzene, cumene, n-propylbenzene, n-butylbenzene, isobutylbenzene, p-cymene, t-butylbenzene, sec-butylbenzene, bromobenzene, bromomethylbenzenes (individual isomers or mixtures thereof), bromodimethylbenzenes (individual isomers or mixtures thereof), chlorobenzene, chlorodimethylbenzenes (individual isomers or mixtures thereof), chloromethylbenzenes (individual isomers or mixtures thereof), diglyme, dioxane, oxepane, di-n-butyl ether, di-sec-butyl ether, DMF, DMAC, isopropyl acetate, isobutyl acetate, n-propylacetate, ethyl n-buyrate, or propionitrile. Of the foregoing solvents, those that boil at or above 110° C. are preferred.

The reaction of Step E can be conducted by mixing (typically dissolving) Compounds VIa and/or VIb or by dissolving Compound VIc in the selected solvent, and then bringing the resulting mixture (typically a solution) to reaction temperature (either under pressure in an autoclave or at atmospheric pressure) and maintaining the mixture at reaction temperature (optionally with agitation such as stirring) until the reaction is complete or the desired degree of conversion is achieved. The reaction time can vary widely depending upon, inter alia, the reaction temperature and the selected reactant and solvent, but the reaction time for complete conversion is typically in a range of from about 2 to about 48 hours (e.g., from about 6 to about 18 hours). Compound VII can subsequently be isolated and redissolved for use in Step F, or the reaction mixture containing Compound VII can be concentrated and then solvent switched for use in Step F.

The present invention includes a process for preparing a compound of Formula X which comprises Steps E, F1, F2, G and H as described above; and which further comprises:

(D) reacting an amidine of Formula (V): 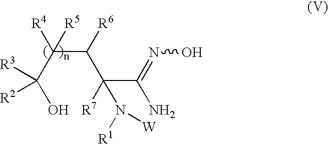
with (i) a mixed dialkyl acetylene dicarboxylate of formula: R^{A}O₂C—≡—CO₂R^{B} to obtain a mixture of compounds of Formula VIa and VIb, or (ii) with a dialkyl acetylene dicarboxylate of formula R^{C}O₂C—≡—CO₂R^{C} to obtain a compound of Formula VIc.

The present invention includes a process for preparing a compound of Formula XI which comprises Steps E, F1-1, F1-2, F2-1, G-1 and H-1 as described above; and which further comprises Step D as described above.

Step D can be conducted in a solvent D. Suitable solvents include those selected from the group consisting of alcohols, ethers, esters, and nitrites. A description of these solvent classes is provided above in the discussion of solvents suitable for use as solvent H in Step H. This description is applicable here with respect to solvents suitable for use as solvent D and is incorporated herein by reference.

The reaction of Step D can be conducted at any temperature at which formation of Compounds VIa, VIb, or VIc can be detected. The temperature is suitably in a range of from about −45 to about 200° C., is typically in a range of from about −10 to about 150° C., and is more typically in a range of from about zero to about 100° C. (e.g., from about 10 to about 50° C.).

The acetylene dicarboxylate can be employed in Step D in any proportion with respect to Compound V which will result in the formation of at least some of Compound VIa, VIb, and/or VIcIX, but it is typically employed in an amount that can optimize conversion to desired compound. The acetylene dicarboxylate is suitably employed in an amount of at least about 0.5 equivalent per equivalent of Compound V, is typically employed in an amount of at least about 0.8 equivalent (e.g., in a range of from about 0.8 to about 30 equivalents, or in a range of from about 0.9 to about 5 equivalents) per equivalent of Compound V, and is more typically employed in an amount of at least about 1 equivalent (e.g., in a range of from about 1 to about 1.5 equivalents per equivalent of Compound V).

The reaction of Step D can be conducted by forming a mixture (typically a solution) of arnidine V in a suitable organic solvent at a temperature below or at the desired reaction temperature, then adding the acetylene dicarboxylate thereto, and then bringing the resulting mixture to reaction temperature and/or maintaining the nixture at reaction temperature (optionally with agitation such as stirring) until the reaction is complete or the desired degree of conversion of amidine V is achieved. The reaction time can vary widely depending upon, inter alia, the reaction temperature and the choice and relative amounts of amidine V and acetylene dicarboxylate, but the reaction time for complete conversion is typically in a range of from about 1 to about 48 hours (e.g., from about 2 to about 24 hours). The Compound VI product can subsequently be isolated from the reaction mixture using conventional procedures and then redissolved for use in Step E, or the reaction mixture containing the compound(s) of Formula VI can be concentrated and then solvent switched for use in Step E.

The present invention includes a process for preparing a compound of Formula X which comprises Steps D, E, F1, F2, G and H as described above; and which further comprises:

(C) reacting hydroxylamine or an acid salt thereof with a protected aminonitrile of Formula IV: 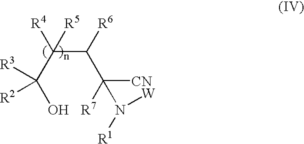
to obtain the amidine of Formula V.

The present invention includes a process for preparing a compound of Formula XI which comprises Steps D, E, F1-1, F1-2, F2-1, G-1 and H-1 as described above; and which further comprises Step C as described above.

The hydroxylamine or its acid salt can suitably be employed in Step C in the form of an aqueous solution, such as a 50% aqueous solution of hydroxylamine. Suitable acid salts include the acid halide salts, such as the hydrochloride or hydrobromide salt of hydroxylamine. The hydroxylamine or its acid salt can be employed in Step C in any proportion with respect to Compound IV which will result in the formation of at least some of Compound V, but it is typically employed in an amount that can optimize conversion to desired compound. The hydroxylamine or its acid salt is suitably be employed in an amount of at least about 0.5 equivalent per equivalent of Compound V, is typically employed in an amount of at least about 0.8 equivalent (e.g., in a range of from about 0.8 to about 100 equivalents) per equivalent of Compound IV, and is more typically employed in an amount of at least about 1 equivalent (e.g., in a range of from about 1 to about 10 equivalents per equivalent of Compound IV, or in a range of from about 1.1 to about 2 equivalents per equivalent of Compound IV).

Step C can be conducted in a solvent C. Suitable solvents include those selected from the group consisting of alcohols and ethers. A description of these solvent classes is provided above in the discussion of solvents suitable for use as solvent H in Step H or as solvent H-1 in Step H-1. This description is applicable here with respect to solvents suitable for use as solvent C and is incorporated herein by reference.

Solvent C can also be a polar organic solvent optionally in admixture with water as a co-solvent. The water can suitably comprise from about 1 to about 90 volume percent of the solvent based on the total volume of solvent. When water is employed as a co-solvent, it is typically employed in an amount in a range of from about 5 to about 50 volume percent based on the total volume of solvent, and is more typically employed in an amount of from about 5 to about 25 vol. % (e.g., from about 5 to about 15 vol. %). The source of co-solvent water can be the hydroxylamine reagent which, as noted above, is suitably employed in the form of an aqueous solution (e.g., 50% hydroxylamine). In one embodiment, solvent C comprises a C₁₋₆ alkyl alcohol and optionally water as a co-solvent. In an aspect of this embodiment, co-solvent water is employed in an amount of from about 5 to about 25 vol. % based on the total volume of solvent. In another aspect of this embodiment, the alcohol is methanol, ethanol, n-propanol, isopropanol, n-butanol, sec-butanol, or isobutanol. In a feature of the preceding aspect, the solvent includes water as a co-solvent in an amount of from about 5 to about 25 vol. % (e.g., from about 5 to about 15 vol. %).

The reaction of Step C can be conducted at any temperature at which formation of amidine V can be detected. The temperature is suitably in a range of from about −10 to about 180° C., is typically in a range of from about zero to about 100° C., and is more typically in a range of from about 30 to about 80° C.

The reaction of Step C can be conducted by forming a mixture (typically a solution) of protected arninonitrile IV in a suitable organic solvent at a temperature below the desired reaction temperature, adding the hydroxylamine thereto, and then bringing the resulting mixture to reaction temperature and maintaining the mixture at reaction temperature (optionally with agitation such as stirring) until the reaction is complete or the desired degree of conversion of aminonitrile IV is achieved. The reaction time can vary widely depending upon, inter alia, the reaction temperature and the relative amounts of aminonitrle IV and hydroxylamine, but the reaction time for complete conversion is typically in a range of from about 0.5 to about 24 hours (e.g., from about 1 to about 12 hours). Amidine V can subsequently be isolated from the reaction mixture using conventional procedures (e.g., distillation or chromatography) and then redissolved for use in Step D, or the reaction mixture containing amidine V can be concentrated and then solvent switched for use in Step D.

The present invention includes a process for preparing a compound of Formula X which comprises Steps C, D, E, F1, F2, G and H as described above; and which further comprises:

(A) treating a cyclic ether of Formula I: 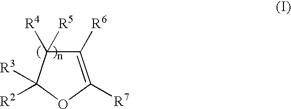
with an aqueous solution of a protonic acid to form an aqueous product mixture comprising a ketohydroxy compound of Formula II: 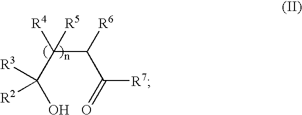
neutralizing the aqueous product mixture and then contacting the neutralized product mixture with an amine of formula R¹NH₂, or an acid salt thereof, and a cyanide reagent to obtain the aminonitrile of Formula (III):

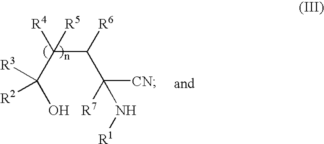

(B) treating the aminonitrile of Formula III with an amiine protecting agent to obtain the protected arninonitrile of Formula IV.

The present invention includes a process for preparing a compound of Formula XI which comprises Steps C, D, E, F1-1, F1-2, F2-1, G-1 and H-1 as described above; and which further comprises Steps A and B as described above.

The cyclic ethers of Formula I employed in Step A above can be prepared in accordance with procedures set forth in, for example, Kukovinets et al., Russ J. Org. Chem. 2001, 37: 235-237; Paquette et al., J. Org. Chem. 1996, 61: 1119-1121; and Wang et al., Tetrahedron Lett. 1993,34: 4881-4884.

The ketohydroxy compound II can be in an equilibrium in the aqueous product mixture with a compound of Formula IIa: 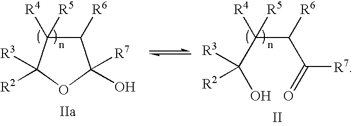
Accordingly, it is understood that Step A of the process of the invention includes the case where the aqueous product mixture comprises Compound II alone or in a mixture with Compound IIa. Any reference herein to Compound II can alternatively be read as a reference to a mixture of Compound II and IIa.

The protonic acid employed in Step A can be a mineral acid or an organic acid. Suitable mineral acids include sulfuric acid, the hydrohalic acids (i.e., HCl, HBr, HI, and HF), nitric acid, phosphoric acid, perchloric acid, periodic acid, and pyrophosphoric acid. Suitable organic acids include carboxylic acids and sulfonic acids, such as C₁₋₆ alkylcarboxylic acids, C₁₋₆ haloalkylcarboxylic acids, C₁₋₆ alkylsulfonic acids, C₁₋₆ haloalkylsulfonic acids, and arylsulfonic acids. Representative examples of organic acids suitable for use in Step A include acetic acid, trifluoroacetic acid (TFA), trichloroacetic acid, toluenesulfonic acid, benzenesulfonic acid, methanesulfonic acid, and trifluoromethanesulfonic acid.

The protonic acid is suitably employed in Step A in a catalytic amount. Accordingly, the amount of catalyst employed in Step A can suitably be an sub-stoichiometric amount in a range of from about 0.001 to less than 1 mole (e.g., from about 0.005 to about 0.5 mole) per mole of cyclic ether I, or an amount in a range of from about 0.01 to about 0.3 mole (e.g., from about 0.05 to about 0.2 mole) per mole of cyclic ether I.

The protonic acid can also be employed in an amount in excess of a catalytic amount or in a range covering catalytic to excess amounts of acid. Accordingly, the protonic acid can suitably be employed in an amount in a range of from about 0.001 to about 150 equivalents per equivalent of cyclic ether I. The protonic acid is typically employed in an amount in a range of from about 0.01 to about 5 equivalents per equivalent of cyclic ether I, and is more typically employed in an amount in a range of from about 0.05 to about 0.5 equivalents per equivalent of cyclic ether I.

The acid treatment in Step A can be conducted at any temperature at which formation of the ketohydroxy compound IE can be detected. The temperature is suitably in a range of from about zero to about 180° C., is typically in a range of from about zero to about 150° C. (e.g., in a range of from about 10 to about 100° C.), and is more typically in a range of from about 10 to about 50° C. (e.g., in a range of from about 20 to about 50° C.).

The aqueous product mixture containing ketohydroxy compound II can be neutralized (i.e., adjusted to a pH in a range of from about 5 to about 10, preferably to a pH in a range of from about 6 to about 8, and more preferably to a pH of about 7) by addition of a suitable proportion of an inorganic or organic base. An objective of the neutralization is to avoid the formation of HCN upon the subsequent addition of the cyanide reagent. Suitable inorganic bases include ammonium hydroxide and metal hydroxides, particularly alkali metal hydroxides such as NaOH and KOH. Suitable organic bases include alkoxides such as alkali metal alkoxides (e.g., alkali metal salts of C₁₋₆ alkyl alcohols such as the methoxides, ethoxides, n-propoxides, and isopropoxides of Li, Na, and K). Primary, secondary, and tertiary amines (e.g., tri-C₁₋₆ alkylamines) are also suitable organic bases. In one embodiment, the aqueous product mixture is neutralized with R¹NH₂; i.e., the same amine subsequently employed in Step A in the conversion of Compound II to aminonitrile III (the Strecker reaction).

The neutralization can be conducted at any temperature at which the neutralization can be detected, is suitably conducted at a temperature in a range of from about −10 to about 50° C., and is typically conducted at a temperature in a range of from about zero to about 30° C.

The neutralized product mixture is contacted with an amine of formula R¹NH₂, or an acid salt thereof, and a cyanide reagent to form aminonitrile III. The variable R¹ is as defined and described above in the discussion of Step H. Acid salts of the amine suitable for use in Step A include mineral acid salts such as salts of the hydrohalic acids, sulfuric acid, nitric acid, and phosphoric acid.

Cyanide reagents suitable for use in Step A include those selected from the group consisting of alkali metal cyanides and trihydrocarbylsilyl cyanides. A class of suitable cyanide reagents consists of reagents selected from the group consisting of LiCN, NaCN, KCN, and trialkylsilyl cyanides of formula (Rˆ)₃SiCN, wherein each Rˆ is independently C₁₋₆ alkyl. Representative examples of trialkylsilyl cyanides suitable for use in Step A include trimethylsilyl cyanide (TMSCN), triethylsilyl cyanide, and tri-n-propylsilyl cyanide.

The cyanide reagent can be employed in Step C in any proportion with respect to Compound I which will result in the formation of at least some of Compound III, but it is typically employed in an amount that can optimize conversion to the desired compound. The cyanide reagent is suitably be employed in an amount of at least about 0.5 equivalent (e.g., in a range of from about 0.5 to about 20 equivalents) per equivalent of Compound III, is typically employed in an amount of at least about 0.8 equivalent (e.g., in a range of from about 0.8 to about 3 equivalents) per equivalent of Compound III, and is more typically employed in an amount of at least about 0.9 equivalent (e.g., in a range of from about 0.95 to about 2 equivalents) per equivalent of Compound in. It is particularly suitable to employ the cyanide reagent in an amount of at least about 1 equivalent (e.g., in a range of from about 1 to about 1.5 equivalents) per equivalent of Compound III.

The amine of formula R¹NH₂ or its acid salt is suitably employed in a molar amount equal to or in excess of the cyanide reagent, is typically employed in an amount of from about 1 to about 20 moles per mole of the cyanide reagent, and is more typically employed in an amount of from about 1 to about 10 moles (e.g., from about 1 to about 5 moles) per mole of the cyanide reagent. (Note: Reference is made here only to the amount of amine involved in the reaction with the cyanide reagent. An additional amount of the amine could be used in the prior neutralization step.) The reaction of the cyanide reagent and the amine of formula R¹NH₂ with the neutralized product mixture can be conducted at any temperature at which formation of aminonitrile III can be detected. The temperature is suitably in a range of from about −10 to about 120° C., is typically in a range of from about zero to about 150° C., is more typically in a range of from about 10 to about 100° C., and is even more typically in a range of from about 20 to about 60° C.

Step A can be conducted by adding the cyclic ether I (either neat or in a suitable solvent such as an alcohol or a halogenated alkane) to the protonic acid (e.g., an aqueous solution of a mineral acid such as sulfuric acid), bringing the resulting mixture to the desired reaction temperature and maintaining the mixture at reaction temperature (optionally with agitation such as stirring) until the reaction is complete or the desired degree of conversion to Compound II is achieved. The reaction time can vary depending upon, inter alia, the reaction temperature and the relative amount of acid employed, but the reaction time for complete conversion is typically in a range of from about 0.5 to about 12 hours. The acidic aqueous product mixture containing ketohydroxy compound HI can then be neutralized by bringing the mixture to a temperature suitable for neutralization, and then slowly adding the selected base to the product mixture (optionally with agitation such as stirring) while maintaining the mixture at the neutralization temperature until the product mixture attains a pH in a range of from about 5 to about 10 (preferably from from about 6 to 8, and more preferably about 7). The pH of the mixture can be monitored during the addition of the base with a pH meter or pH paper. Following neutralization, the cyanide reagent and the R¹NH₂ amine can then be added to the neutralized product mixture, and the resulting admixture aged at a suitable reaction temperature until the reaction to aminonitrile is completed. The reaction time can vary depending upon, inter alia, the reaction temperature and the choice and relative amounts of reactants, but the reaction time for complete conversion is typically in a range of from about 2 to about 96 hours. Aminonitrile m can subsequently be isolated from the reaction mixture using conventional procedures (e.g., distillation or chromatography) and then redissolved for use in Step B, or the reaction mixture containing aminonitrile III can be extracted with a suitable organic solvent (e.g., an ester) and the extract concentrated for use in Step B.

In Step B the aminonitrile of Formula III is treated with an amine protecting agent to obtain the protected aminonitrile of Formula IV. As indicated above in the description of Step H, the amine protective group W in Compound IV can be any amine protective group that is sufficiently stable to survive the reactions set forth in Steps C to H and labile enough to be removed (cleaved) from Compound X or derivatives thereof (e.g., Compound XI as described below) via contact with a suitable amine deprotecting agent to give the free amino group with little or no degradation of other functional groups which may be present. Amine protecting agents suitable for use in Step B include the agents selected from the group consisting of:

(i) compounds of formula W-Q, wherein Q is halide and W is:

(1) C₁₋₆ alkyl substituted with aryl,
(2) C(═O)—C₁₋₄ alkyl,
(3) C(═O)—C₁₋₄ haloalkyl,
(4) C(═O)—C₁₋₄ alkylene-aryl,
(5) C(═O)—O—C₁₋₄ alkyl,
(6) C(═O)—O—(CH₂)₀₋₁—CH═CH₂, or
(7) C(═O)—O—C₁₋₄ alkylene-aryl; and

(ii) anhydrides of formula (W)₂O, wherein W is —C(═O)—O—C₁₋₄ alkyl, —C(═O)—O—C₁₋₄ alkylene-aryl, or —C(═O)—O—CH₂)₀₋₁CH═CH₂;

wherein any aryl in a group defmed in (i) or (ii) is optionally substituted with from 1 to 5 substituents each of which is independently halo, —NO₂, —C₁₋₄ alkyl, or —O—C₁₋₄ alkyl; and

wherein the treatment results in the attachment of group W to aminonitrile III to obtain Compound IV.

A class of amine protecting agents suitable for use in Step B consists of i) agents of formula W-Q, wherein Q is: (1) —CH₂-phenyl, (2) —C(═O)—C₁₋₄ alkyl, (3) —C(═O)—CF₃, (4) —C(═O)—CCl₃, (5) —C(═O)—CH₂-phenyl, (6) —C(═O)—O—C₁₋₄ alkyl, (7) —C(═O)—O—CH₂—CH═CH₂, and (8) —C(═O)—O—CH₂-phenyl; wherein any phenyl in a group defined above is optionally substituted with from 1 to 3 substituents each of which is independently halo, —NO₂, —C₁₋₄ alkyl, or —O—C₁₋₄ alkyl; and ii) agents of formula (W)₂O, wherein W is BOC, CBZ, or ALLOC. A sub-class of this class consists of amine protecting agents selected from BOC-Q and (BOC)₂O.

Representative examples of amine protecting agents suitable for use in Step B includes BOC—Cl, CBZ-Cl, (CBZ)₂O, (ALLOC)₂O, allyl chloroformate, and (BOC)₂O.

Further description of the foregoing agents and of other amine protecting agents suitable for use in Step B can be found in Protective Groups in Organic Chemistry, edited by J. F. W. McOmie, Plenum Press, New York, 1973, pp. 43-74; and in T. W. Greene and P. G. M. Wuts, Protective Groups in Organic Synthesis, 2^{nd} edition, John Wiley, New York, 1991, pp. 309-385.

The amine protecting agent is typically employed in an amount that can optimize conversion of aminonitrile III to protected aminonitrile IV. The amine protecting agent is suitably employed in an amount in a range of from about 0.9 to about 10 equivalents per equivalent of aminonitrile III, and is typically employed in an amount in a range of from about 0.9 to about 3 (e.g., from about 1.05 to about 3) equivalents per equivalent of aminonitrile III.

The treatment in Step B can be conducted at any temperature at which the reaction to form Compound IV can be detected. The temperature is suitably in a range of from about −20 to about 100° C., and is typically in a range of from about −20 to about 60° C. (e.g., from about −5 to about 50° C.).

Step B can be conducted in solvent B. Suitable solvents include aromatic hydrocarbons, halogenated alkanes, halogenated cycloalkanes, alcohols, esters, ethers, and nitriles. Further description of these solvent classes is set forth above in the discussion of solvents suitable for use in Step F1, Step H, and other steps. These earlier descriptions are applicable here, and are incorporated herein by reference.

Step B can be conducted by adding the amnine protecting agent to a mixture (typically a solution) of aminonitrile III and solvent, bringing the resulting mixture to the desired reaction temperature and maintaining the mixture at reaction temperature (optionally with agitation such as stirring) until the reaction is complete. The reaction time can vary depending upon, inter alia, the reaction temperature and the relative amount of amine protecting agent employed, but the reaction time for complete conversion is typically in a range of from about 0.5 to about 12 hours. The protected aminonitrile IV can subsequently be isolated from the reaction mixture using conventional procedures and then redissolved for use in Step C, or the reaction mixture containing IV can be concentrated and then solvent switched for use in Step C.

The present invention also includes a process for preparing a compound of Formula XII: 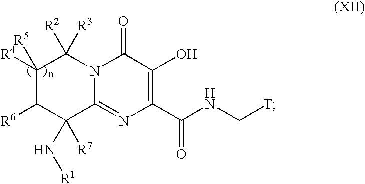
which comprises conducting Step H as described above, and which further comprises:

(I) reacting an amine of formula T-CH₂NH₂ with a compound of Formula X to obtain a compound of Formula XI; and

(J) treating the carboxarnide XI with an amine deprotecting agent to remove group W and obtain a compound of Formula XII; further optionally comprises:

(I^{a}) (i) reacting a compound of Formula XI with a hydroxy activating agent to form a racemic compound of Formula XIa:

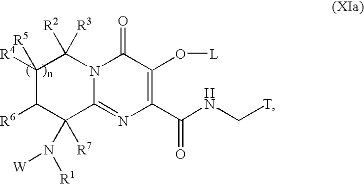

(ii) treating a compound of Formula XIa with an amine deprotecting agent to remove group W and obtain a compound of Formula XIIa:

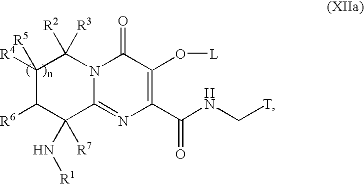

(iii) converting a racemic compound of Formula XIIa to an enantiomerically-enriched form wherein the amount of (S)-Compound XIIa is greater than the amount of (R)-Compound XIIa, and

(iv) removing the L group from the enantiomerically-enriched form of Compound XIIa; or (J^{a}) converting a racemic compound of Formula XII to an enantiomerically-enriched form wherein the amount of (S)-Compound XII is greater than the amount of (R)-Compound XII.

The present invention also includes a process for preparing a compound of Formula XII which comprises conducting Step H-1 as described above; and which further comprises conducting optional Step I^{a}, Step J, and optional Step J^{a}.

Step I concerns the coupling of Compound X with an amine of formula T-CH₂NH₂ to obtain Compound XI. The coupling reaction is suitably conducted in solvent at a temperature in the range of from about 40 to about 200° C., and is typically conducted at a temperature in the range of from about 50 to about 160° C. In one embodiment, the coupling reaction is conducted at solvent reflux at atmospheric pressure, wherein the solvent is chosen to provide the desired reflux temperature. Solvents suitable for use in Step I include those selected from the group consisting of alkanes, cycloalkanes, aromatic hydrocarbons, halogenated alkanes, halogenated cycloalkanes, alcohols, esters, ethers, and nitrites. Further description of these solvent classes is set forth above in the discussion of solvents suitable for use in Step F1, Step H, and other steps. These earlier descriptions are applicable here, and are incorporated herein by reference. A class of solvents suitable for use in Step I consists of those selected from the group consisting of alcohols, esters and ethers. A sub-class of this class consists of the solvents selected from the group consisting of C₁-C₆ alkyl alcohols, dialkyl ethers wherein each alkyl is independently a C₁-C₄ alkyl, C₄-C₅ cyclic ethers, and C₁-C₄ alkyl esters of C₁-C₄ alkylcarboxylic acids. Another sub-class of this class is a solvent selected from the group consisting of methanol, ethanol, n-propanol, isopropanol, t-butyl alcohol, diethylether, 1,2-dimethoxyethane, THF, methyl acetate, ethyl acetate, and isopropyl acetate.

The amine of formula T-CH₂NH₂ can be employed in Step I in any proportion which will result in the formation of at least some of Compound XI. Typically, however, the reactants are employed in proportions which can optimize conversion of at least one of the reactants, and usually the amine is employed in an amount that can optimize the conversion of Compound X. The amine can be suitably employed in an amount of at least about 0.5 equivalent (e.g., in a range of from about 0.5 to about 10 equivalents) per equivalent of Compound X. It is preferred to use an excess of amine in order to increase the degree of conversion and/or shorten the reaction time. Accordingly, the amine is typically employed in an amount of at least about 1.05 equivalents per equivalent of Compound X, and is more typically employed in an amount in a range of from about 1.1 to about 10 equivalents, or from about 2 to about 10 equivalents, or from about 2 to about 5 equivalents, or from about 2.5 to about 3.5 equivalents (e.g., about 3 equivalents), per equivalent of Compound X.

The reaction of Step I can be suitably conducted by adding the amine of formula T-CH₂NH₂ to a solution or suspension of Compound X in the selected solvent and then heating the mixture to reaction temperature and maintaining at reaction temperature until the reaction is complete or the desired degree of conversion of the reactants is achieved. Isolation of the amide product XI can be accomplished using conventional procedures, and the isolated product can be re-dissolved for use in Step J. Alternatively the reaction mixture containing product XI can be used directly in Step J.

In Step J, the carboxamide of Formula XI is treated with an amine deprotecting agent that can remove W to obtain a carboxamide of Formula XII. Suitable W groups have already been described above. (see, e.g., the description of Step B and Step H), and include alkyloxycarbonyls (e.g., BOC), arylmethyloxycarbonyls (e.g., CBZ), and allyloxycarbonyl (ALLOC). These W groups can be formed in the manner described above in the description of Step B. In most instances the W groups can be removed by treatment with acids including mineral acids, Lewis acids, and organic acids. Suitable mineral acids include hydrogen halides (HCl, HBr, and HF, as a gas or in aqueous solution), sulfuric acid, and nitric acid. Suitable organic acids include carboxylic acids, alkylsulfonic acids and arylsulfonic acids. Exemplary organic acids include trifluoroacetic acid (TFA), toluenesulfonic acid, benzenesulfonic acid, methanesulfonic acid, and trifluoromethanesulfonic acid. Suitable Lewis acids include BF₃.Et₂O, SnCl₄, ZnBr₂, Me₃SiI, Me₃SiCl, Me₃SiOTf, and AlCl₃. Cleavage conditions (e.g., temperature, choice and concentration of acid) can vary from mild to harsh depending upon the lability of the amino protective group. Suitable solvents include AcOEt, MeOH and AcOEt/MeOH. In one embodiment the temperature is in a range of from about 15 to about 110° C., and the acid is present in an amount of at least about 1 equivalent (e.g., in a range of from about 1 to about 10 equivalents) per equivalent of Compound XI. Although acid treatment is typically effective, other means can often be employed. Removal of CBZ or ALLOC, for example, is typically accomplished via hydrogenolysis (e.g., hydrogenation with a Pd catalyst). Further description of amine deprotecting agents and deprotection treatments suitable for use in Step J can be found in Protective Groups in Organic Chemistry, edited by J. F. W. McOmie, Plenum Press, New York, 1973, pp. 43-74; and in T. W. Greene and P. G. M. Wuts, Protective Groups in Organic Synthesis, 2^{nd} edition, John Wiley, New York, 1991, pp. 309-385. After removal of the protective group, Compound XII can be isolated using conventional techniques.

It is noted that under the reaction conditions employed in Step I, the L group is typically removed (cleaved) to afford a hydroxy group. In particular, when L is a sulfonyl or phosphinyl ester group, it is generally removed during the amine coupling of Step I to afford Compound XI. In the event the L group is chemically stable during amine coupling in Step I, then L can be removed separately or concurrently with the removal of group W in Step J to obtain Compound XII. Generally speaking, a chemical treatment can be employed in Step J which is suitable both for the removal of group W (e.g., hydrogenolysis or acid hydrolysis as described above) and of any residual L.

Optional Step I^{a} and optional Step J^{a} relate to optical resolution of racemic forms of Compounds XII. Generally, racemates of the present invention may be resolved into enantiomerically-enriched forms, typically with more than 50% enantiomeric excess (“ee”), more typically with more than 70% ee, and most typically with more than 90% ee, where the amount of one enantiomer is greater than that of the other enantiomer (e.g., the amount of (S)-Compound XII is greater than the amount of (R)-Compound XII). Such resolution/conversion can be realized by techniques known to one skilled in the art. Examples of such techniques include resolution by means of diastereomeric salts, enzymes as resolving agents, high-performance liquid chromatography using chiral stationary phases, and ligand-exchange capillary electrophoresis using chiral selectors. In optional Step la, the hydroxy group of the racemnic Compound XI is first converted to —OL before the amine protecting group W is removed. The resulting racemic Compound XIIa is then undergone optical resolution. The L group of Compound XIIa may be removed by methods described above for removal of L groups. In Step J^{a}, the racemic Compound XII is converted to enantiomerically-enriched forms by optical resolution. Suitable enantiomerically pure chiral resolving agents include di-p-toluoyl-D-tartaric acid (D-DTTA) and di-p-toluoyl-L-tartaric acid (L-DTTA). Suitable solvents used in the optical resolution process include DMF. It should be noted that analogous optical resolution steps may be incorporated into other appropriate steps of the present processes to obtain enantiomerically pure compounds of this invention.

Embodiments of the process for preparing Compound XII include the process as described above and further comprising one or more of the pre-steps described above for preparing Compound X or XI. Thus, embodiments of the process include the process comprising Steps H, I, J and optional I^{a} or J^{a}; and (1) further comprising Steps F1, F2 and optional Step G, or (2) further comprising Steps E, F1, F2 and optional Step G, or (3) further comprising Steps D, E, F1, F2 and optional Step G, or (4) further comprising Steps C, D, E, F1, F2 and optional Step G, or (5) further comprising Steps A, B, C, D, E, F1, F2 and optional Step G. Other embodiments of the process include the process comprising Steps H-1, J and optional I^{a}or J^{a}; and (1) further comprising Steps F1-1, F1-2, F2-1 and optional Step G-1, or (2) further comprising Steps E, F1-1, F1-2, F2-1 and optional Step G-1, or (3) further comprising Steps D, E, F1-1, F1-2, F2-1 and optional Step G-1, or (4) further comprising Steps C, D, E, F1-1, F1-2, F2-1 and optional Step G-1, or (5) further comprising Steps A, B, C, D, E, F1-1, F1-2, F2-1 and optional Step G-1.

The present invention also includes a process for preparing a compound of Formula XIII: 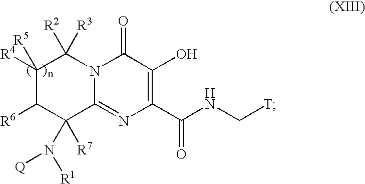
which comprises conducting Step H, Step I and Step J as described above; and which further comprises:

(K) treating the compound of Formula XII with Q-Z to obtain the compound of Formula XIII; wherein Q is:
(1) C(═O)R^{D},
(2) SO₂R^{D},
(3) C(═O)OR^{E}, or
(4) R^{E}, provided that Z is halo,
wherein
   R^{D} is C₁₋₆ alkyl, C₁₋₆ fluoroalkyl, aryl, HetB, or —C₁₋₄ alkylene-NR^{M}R^{N};
   R^{E} is C₁₋₆ alkyl;
   HetB is a 5- or 6-membered heteroaromatic ring containing from 1 to 4 heteroatoms independently selected from N, O and S, wherein the heteroaromatic ring is optionally substituted with 1 or 2 C₁₋₆ alkyl groups;
   R^{M} and R^{N} are each independently C₁₋₆ alkyl or C₁₋₆ alkyl substituted with aryl, or alternatively RM and RN together with the N to which they are both attached form C₄₋₇ azacycloalkyl; and
      Z is halo, OH, OC(═O)—O—C₁₋₄ alkyl, OC(═O)—C(CH₃)₃, or OP(═O)(phenyl)₂.

The present invention also includes a process for preparing a compound of Formula XIII which comprises conducting Step H-1 and Step J as described above; and which further comprises conducting Step K.

Step K involves derivatizing (i.e., acylating, sulfonylating, or alkylating) the free amino group in Compound XII to form Compound XIII. The coupling reaction is suitably conducted in solvent at a temperature in the range of from about 40 to about 200° C., and is typically conducted at a temperature in the range of from about 50 to about 160° C. Solvents suitable for use in Step K include those selected from the group consisting of halogenated alkanes, halogenated cycloalkanes, ethers, and nitriles. Further description of these solvent classes is set forth above in the discussion of solvents suitable for use in Step F1, Step F1-2, Step H, Step H-1 and other steps. These earlier descriptions are applicable here, and are herein incorporated.

The reagents of formula Q-Z are either available commercially or can be prepared by methods known in the art. The reagent Q-Z can be employed in Step K in any proportion which will result in the formation of at least some of Compound XIII. Typically, however, Q-Z is employed in a stoichiometric or excess amount (i.e., an amount greater than about 1 equivalent per equivalent of Compound XII) in order to optimize the conversion of Compound XII. Q-Z is typically employed in an amount of at least about 1.05 equivalents per equivalent of Compound X, and is more typically employed in an amount in a range of from about 1.1 to about 10 equivalents per equivalent of Compound X. The reaction of Step K can be suitably conducted by adding Q-Z to a solution or suspension of Compound XII in the selected solvent or by adding Compound XII (either as a solid or in solution) to a solution or suspension of Q-Z, and then heating the mixture to reaction temperature and maintaining at reaction temperature until the reaction is complete or the desired degree of conversion of the reactants is achieved. Isolation of Compound XIII can be accomplished using conventional procedures.

Embodiments of the process for preparing Compound XIII include the process as described above and further comprising one or more of the pre-steps described above for preparing Compound X or XI. Thus, embodiments of the process include the process comprising Steps H, I, J and K; and (1) further comprising Steps F1, F2 and optional Step G, or (2) further comprising Steps E, F1, F2 and optional Step G, or (3) further comprising Steps D, E, F1, F2 and optional Step G, or (4) further comprising Steps C, D, E, F1, F2 and optional Step G, or (5) further comprising Steps A, B, C, D, E, F1, F2 and optional Step G. Other embodiments of the process include the process comprising Steps H-1, J and K; and (1) further comprising Steps F1-1, F1-2, F2-1 and optional Step G-1, or (2) further comprising Steps E, F1-1, F1-2, F2-1 and optional Step G-1, or (3) further comprising Steps D, E, F1-1, F1-2, F2-1 and optional Step G-1, or (4) further comprising Steps C, D, E, F1-1, F1-2, F2-1 and optional Step G-1, or (5) further comprising Steps A, B, C, D, E, F1-1, F1-2, F2-1 and optional Step G-1. This process may also include optical resolution steps as described above.

The present invention also includes a process for preparing a compound of Formula XIV: 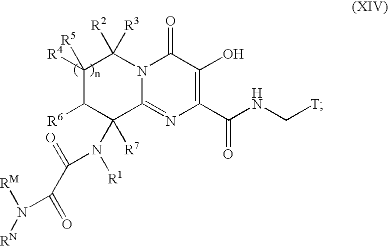
which comprises conducting Step H, Step I and Step J and optional Step I^{a} or Step J^{a} as described above; and which further comprises:

(L) either (i) reacting the compound of Formula XII with (i) (R^{M}R^{N})N—C(═O)—C(═O)—OC(═O)—O—C₁₋₆ alkyl, or (ii) reacting the compound of Formula XII with R^{F}O—C(═O)—C(═O)-Z and then with (R^{M}R^{N})NH, to obtain Compound XIV; wherein R^{M} and R^{N} are each independently C₁₋₆ alkyl or C₁₋₆ alkyl substituted with aryl, or alternatively R^{M} and R^{N} together with the N to which both are attached form C₄₋₇ azacycloalkyl; R^{F} is C₁₋₆ alkyl; and

Z is halo or OH.

The present invention also includes a process for preparing a compound of Formula XIV, which comprises conducting Step H-1 and Step J and optional Step I^{a} or Step J^{a} as described above; and which further comprises conducting Step L.

With respect to reaction (i) of Step L, the reaction temperature, choice of solvents, relative amount of reagent, method of conducting the reaction, etc. are essentially the same as set forth above for Step K, except that Q-Z of Step K is replaced by the reagent (R^{M}R^{N})N—C(═O)—C(═O)—OC(═O)—O—C₁₋₆ alkyl in (i). Similarly the reaction conditions, etc. for reacting R^{F}O—C(═O)—C(═O)-Z in reaction (ii) of Step L parallel those for reacting Q-Z in Step K. The subsequent reaction in (ii) with the amine of formula (R^{M}R^{N})NH is typically conducted by adding the amine to the reaction mixture containing acylated XII, bringing the mixture to the desired reaction temperature and aging the mixture at the reaction temperature until the amidation is complete.

Embodiments of the process for preparing Compound XIV include the process as described above and further comprising one or more of the pre-steps described above for preparing Compound X or XI. Thus, embodiments of the process include the process comprising Steps H, I, J and L and optional Step I^{a} or Step J^{a}; and (1) further comprising Steps F1, F2 and optional Step G, or (2) further comprising Steps E, F1, F2 and optional Step G, or (3) further comprising Steps D, E, F1, F2 and optional Step G, or (4) further comprising Steps C, D, E, F1, F2 and optional Step G, or (5) further comprising Steps A, B, C, D, E, F1, F2 and optional Step G. Other embodiments of the process include the process comprising Steps H-1, J and L and optional Step I^{a} or Step J^{a}; and (1) further comprising Steps F1-1, F1-2, F2-1 and optional Step G-i, or (2) further comprising Steps E, F1-1, F1-2, F2-1 and optional Step G-1, or (3) further comprising Steps D, E, F1-1, F1-2, F2-1 and optional Step G-1, or (4) further comprising Steps C, D, E, F1-1, F1-2, F2-1 and optional Step G-1, or (5) further comprising Steps A, B, C, D, E, F1-1, F1-2, F2-1 and optional Step G-1.

The present invention also includes a process for preparing a compound of Formula XX or Formula XI: 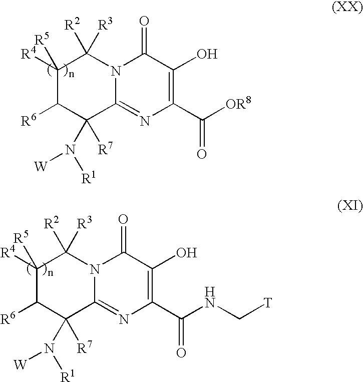
which comprises:

(HZ) treating a compound of Pormula VII or Formula VIII-1: 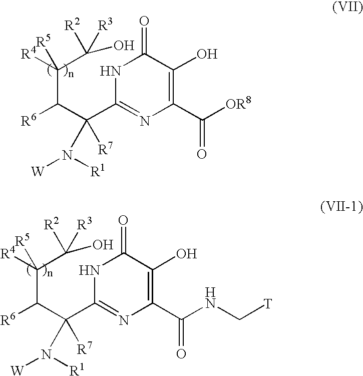
with a trihydrocarbylphosphine reagent in the presence of an azodicarboxylate of Formula R^{Y}O₂C—N═N—CO₂R^{Z} to form the compound of Formula XX or XI, respectively; wherein R^{Y} and R^{Z} are each independently C₁₋₆ alkyl; and W, R¹, R², R³, each R⁴, each R⁵, R⁶, R⁷, R⁸, aryl, T, and n are as originally defined above. It is understood that any one or more of the variables W, R¹, R², R³, R⁴, R⁵, R⁶, R⁷, R⁸, aryl, T, and n can alternatively be as defmed in any embodiment (or aspect thereof) set forth above (see, e.g., the embodiments set forth in the description under Step H and Step H-1), and that each unique set of variable definitions resulting therefrom represents an embodiment of the process for preparing Compound XX or XI.

Another embodiment of the process for preparing compound XX or XI is the process as just defined or as defined in any of the embodiments included in the preceding paragraph, wherein R^{Y} and R^{Z} are each independently C₁₋₄ alkyl; and all other variables are as originally defined or as defined in any preceding embodiments. In an aspect of this embodiment, R^{Y} and R^{Z} are the same C₁₋₄ alkyl group. Representative examples of azidocarboxylates suitable for use in Step HZ include diethylazidodicarboxylate (DEAD) and diisopropylazidodicarboxylate (DIAD).

Another embodiment of the process for preparing compound XX or XI is the process as originally defined just above or as defmed in any of the preceding embodiments, wherein the trihydrocarbylphosphine reagent is a reagent of formula P(R^{X})₃ wherein each R^{X} is independently aryl or C₁₋₆ alkyl. Representative examples of phosphine reagents suitable for use in Step HZ include triphenylphosphine, trimethylphosphine, triethylphosphine, and triisopropylphosphine.

The treatment in Step HZ can be conducted at any temperature at which the formation of Compound XX or XI can be detected. The temperature is suitably in a range of from about −10 to about 40° C., and is typically in a range of from about zero to about 30° C.

The trihydrocarbylphosphine reagent can be employed in Step HZ in any proportion with respect to Compound VII or VII-1 which will result in the formation of at least some of Compound XX or XI, respectively, but it is typically employed in an amount that can optimize conversion to the desired compound. The phosphine reagent is suitably be employed in an amount of at least about 0.5 equivalent per equivalent of Compound VII or VII-1, is typically employed in an amount of at least about 1 equivalent (e.g., in a range of from about 1 to about 1.5 equivalents) per equivalent of Compound VII or VII-1.

The azidocarboxylate is typically employed in an equimolar amount with respect to the phosphine reagent (i.e., about a 1:1 molar ratio of azidocarboxylate to phosphine reagent).

Step HZ can be conducted in solvent. Suitable solvents include those described above as suitable solvents for Step F1 or Step F1-2.

Step HZ can be conducted by mixing (typically dissolving) the trihydrocarbylphosphine reagent and the azidodicarboxylate together in an appropriate solvent, then adding Compound VII or VII-1, then bringing the resulting mixture (typically a solution) to reaction temperature and maintaining the mixture at reaction temperature (optionally with agitation such as stirring) until the reaction is complete or the desired degree of conversion is achieved. The reaction time can vary widely depending upon, inter alia, the reaction temperature and the selected reactants, but the reaction time for complete conversion is typically in a range of from about 1 to about 12 hours. Compound XX or XI can subsequently be isolated using conventional techniques.

The present invention also includes the process for preparing Compound XX or XI which comprises Step HZ for obtaining Compound XX or XI from Compound VII or VII-1, respectively, as described above; and which further comprises:

(i) Step E as described above for obtaining Compound VII from a mixture of Compounds VIa and VIb or from Compound VIc;

(ii) Step E and also Step D as described above for obtaining VIa and Vlb or VIc from an amidine V;

(iii) Steps E and D and also Step C as described above for obtaining amidine V from protected aminonitrile IV; or

(iv) Steps E, D, and C, and also Steps A and B as described above for obtaining the protected aminonitrile IV from cyclic ether I.

It is understood that any embodiment or aspect of any one of these steps can be employed with any embodiment or aspect of any one or more of the other steps (with the understanding of course that the variables appearing in more than one step—e.g., certain variables defining reactants and products in the steps—have consistent definitions).

The present invention also includes a process for preparing a compound of Formula XII which comprises conducting Step HZ as described above; and which further comprises:

(i) when the product of Step HZ is Compound XX,

(IZ) reacting an amine of formula T-CH₂NH₂ with the compound of Formula XX to obtain a carboxamide of Formula XI; and

(JZ) treating the carboxarnide XI with an amine deprotecting agent to remove group W and obtain the compound of Formula XII; and

(ii) when the product of Step HZ is Compound XI, then Step (JZ); wherein Steps IZ and JZ correspond to Steps I and J as previously described.

The present invention also includes a compound of Formula VIIb or VIIb-1: 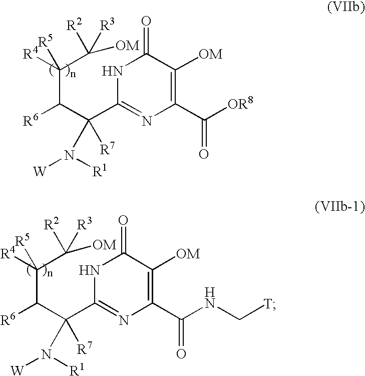
wherein each M is H or a hydroxy activating group; and all other variables are as originally defined above or as defined in any of the preceding embodiments (see, e.g., the embodiments defined in the description of Step H or H-1).

An embodiment is a compound of Formula VIIb or VIIb-1, wherein each M is H or each M is: (1) SO₂R^{I}, (2) P(O)(R^{J})₂, or (3) P(O)(OR^{K})₂; wherein R^{I} is (i) C₁₋₆ alkyl, (ii) C₁₋₆ haloalkyl, (iii) C₁₋₆ alkyl substituted with aryl, (iv) aryl, or (v) camphoryl; each R^{J} is independently (i) C₁₋₆ alkyl, (ii) C₁₋₆ haloalkyl, (iii) C₁₋₆ alkyl substituted with aryl, or (iv) aryl; and each R^{K} is independently (i) C₁₋₆ alkyl or (ii) C₁₋₆ alkyl substituted with aryl; and wherein any aryl defined in R^{I}, R^{J}, and R^{K} is optionally substituted with from 1 to 5 substituents each of which is independently halogen, —C₁₋₄ alkyl, —O—C₁₋₄ alkyl, CF₃, OCF₃, CN, or nitro;

W is: (1) —CH₂-phenyl, where the phenyl is optionally substituted with from 1 to 3 substituents each of which is independently halo, —NO₂, —C₁₋₄ alkyl, or —O—C₁₋₄ alkyl, (2) —C(═O)—C₁₋₄ alkyl, (3) —C(═O)—C₁₋₄ haloalkyl, (4) —C(═O)—CH₂-phenyl, where the phenyl is optionally substituted with from 1 to 3 substituents each of which is independently halo, —NO₂, —C₁₋₄ alkyl, or —O—C₁₋₄ alkyl, (5) —C(═O)—O—C₁₋₄ alkyl, (6) —C(═O)—O—CH₂—CH═CH₂, and (7) —C(═O)—O—CH₂-phenyl, where the phenyl is optionally substituted with from 1 to 3 substituents each of which is independently halo, —NO₂, —C₁₋₄ alkyl, or —O—C₁₋₄ alkyl;

R¹ is C₁₋₆ alkyl or C₁₋₆ alkyl substituted with aryl wherein the aryl is optionally substituted with from 1 to 3 substituents each of which is independently C₁₋₄ alkyl, O—C₁₋₄ alkyl, CF₃, OCF₃, halo, CN, or NO₂;

R², R³, each R⁴, each R⁵, R⁶, and R⁷ are all H; and

T is 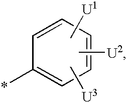
wherein U¹, U² and U³ are each independently H, halo, C₁₋₆ alkyl or C₁₋₆ fluoroalkyl.

In an aspect of the preceding embodiment, the compound of Formula VIIb is Compound 7 or Compound 8:

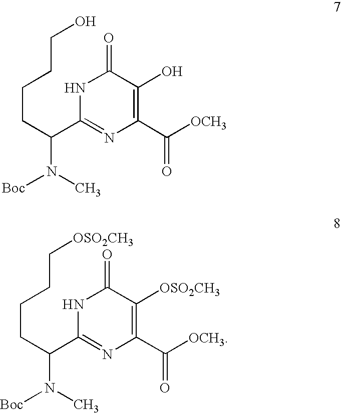

In another aspect of the preceding embodiment, the compound of Formula VIIb-1 is:

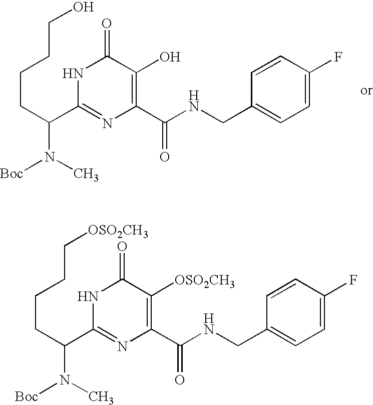

The present invention also includes a compound of Formula VId: 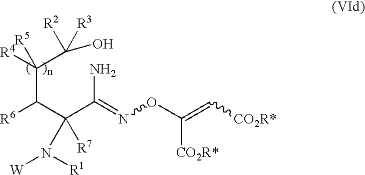
wherein each R* is independently a C₁₋₆ alkyl group; and all other variables are as originally defined above or as defined in any of the preceding embodiments (see, e.g., the embodiments defined in the description of Step H or H-1).

An embodiment is a compound of Formula VId, wherein each R* is the same C₁₋₄ alkyl group; W is (1) —CH₂-phenyl, where the phenyl is optionally substituted with from 1 to 3 substituents each of which is independently halo, —NO₂, —C₁₋₄ alkyl, or —O—C₁₋₄ alkyl, (2) —C(═O)—C₁₋₄ alkyl, (3) —C(═O)—C₁₋₄ haloalkyl, (4) —C(═O)—CH₂-phenyl, where the phenyl is optionally substituted with from 1 to 3 substituents each of which is independently halo, —NO₂, —C₁₋₄ alkyl, or —O—C₁₋₄ alky, (5) —C(═O)—O—C₁₋₄ alkyl, (6) —C(═O)—O—CH₂—CH═CH₂, and (7) —C(═O)—O—CH₂-phenyl, where the phenyl is optionally substituted with from 1 to 3 substituents each of which is independently halo, —NO₂, —C₁₋₄ alkyl, or —O—C₁₋₄ alkyl;

R¹ is C₁₋₆ alkyl or C₁₋₆ alkyl substituted with aryl wherein the aryl is optionally substituted with from 1 to 3 substituents each of which is independently C₁₋₄ alkyl, O—C₁₋₄ alkyl, CF₃, OCF₃, halo, CN, or NO₂; and

R², R³, each R⁴, each R⁵, R⁶, and R⁷ are all H.

In an aspect of the preceding embodiment, the compound of Formula VId is Compound 6:

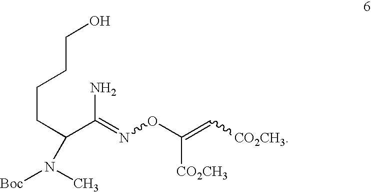

The present invention also includes a compound of Formula V: 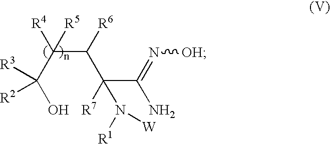
wherein all of the variables are as originally defined above or as defined in any of the preceding embodiments (see, e.g., the embodiments defined in the description of Step H). In one aspect, the compound of Formula V is Compound 5:

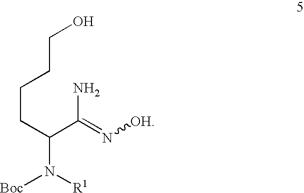

The present invention also includes a compound of Formula III or a compound of Formula IV: 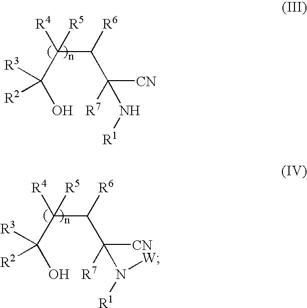
wherein all of the variables are as originally defined above or as defined in any of the preceding embodiments (see, e.g., the embodiments defined in the description of Step H). In one aspect, the compound is Compound 3 or Compound 4:

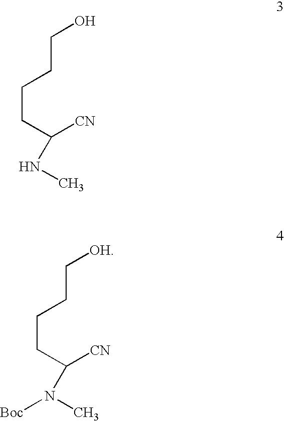

The present invention also includes a process for preparing a compound of Formula X*: 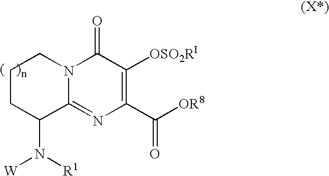
which comprises:

(hh) contacting a compound of Formula VIII*: 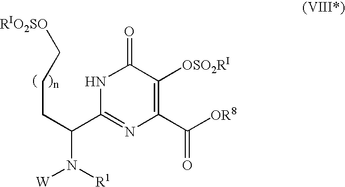
with a strong base to obtain Compound X*; wherein W, R^{I}, R¹, R⁸ and n are each as originally defined above or as defined in any of the preceding embodiments. The reaction conditions, bases, solvents, relative proportions of reactants and reagents, procedures, etc. described above as suitable with respect to 10 Step H are suitable and applicable here to Step hh, and represent embodiments and/or aspects of this process for preparing Compound X*. Another embodiment of this process for preparing Compound X* is a process for preparing Compound 9: 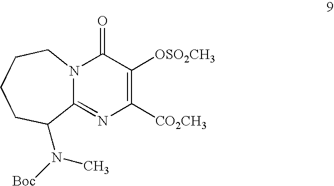
which comprises:

(hh) contacting Compound 8: 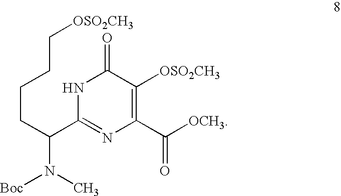
with a strong base to obtain Compound 9.

The present invention also includes a process for preparing a compound of Formula XI*: 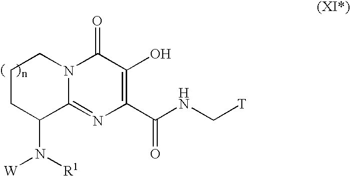
which comprises:

(hh-1) contacting a compound of Formula VIII-1*, VIII-2* or VIII-3*: 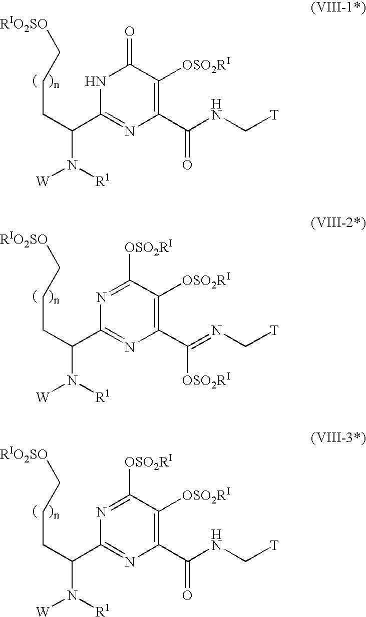
with a strong base to obtain Compound XI*; wherein W, R^{I}, R¹, R⁸, T and n are each as originally defined above or as defined in any of the preceding embodiments. The reaction conditions, bases, solvents, relative proportions of reactants and reagents, procedures, etc. described above as suitable with respect to Step H-1 are suitable and applicable here to Step hh-1, and represent embodiments and/or aspects of this process for preparing Compound XI*. Another embodiment of this process for preparing Compound XI* is a process for preparing Compound 10: 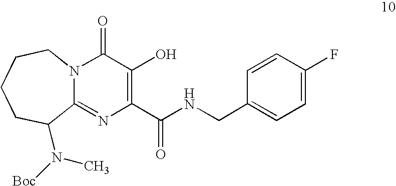
which comprises:

(hh-1) contacting Compound 8-1 and/or Compound 8-2 and Compound 8-3: 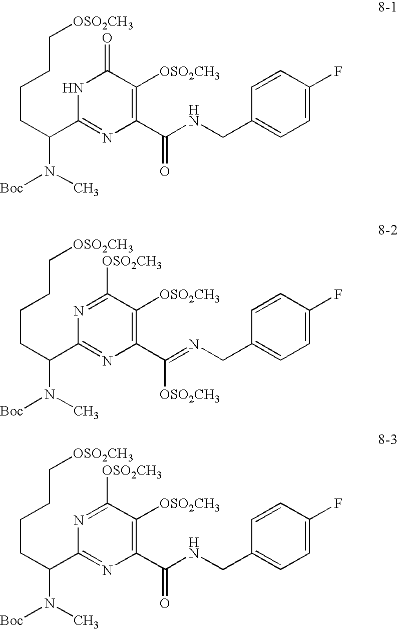
with a strong base to obtain Compound 10.

The present invention also includes a process for preparing a compound of Formula X* which comprises Step hh as described above; and which further comprises:

(ff1) treating a compound of Formula VII*: 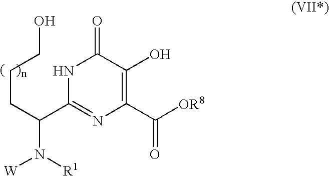
with R^{I}SO₂X, wherein X is halogen, in the presence of a base to form a product which is (i) the compound of Formula VIII*, (ii) a compound of Formula VIIIa*: 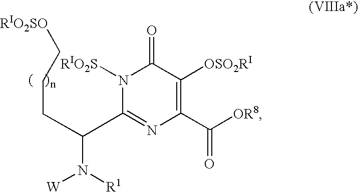
or (iii) a mixture of Compound VIII* and Compound VIIIa*;

(ff2) then:

(1) when the product is (i) Compound VIII*, proceeding directly to Step hh;

(2) when the product is (ii) Compound VIIIa*, contacting the product with (a) a primary or secondary amine or (b) an alcohol, water, or an alcohol-water mixture in the presence of a base, to form Compound VIII*; or

(3) when the product is (iii) a mixture of Compounds VIII* and VIII*, optionally contacting the product with (a) a primary or secondary amine or (b) an alcohol, water, or an alcohol-water mixture in the presence of a base, to form additional Compound VIII*.

The reaction conditions, bases, solvents, relative proportions of reactants and reagents, procedures, etc. described above as suitable with respect to Steps F1 and F2 are suitable and applicable here to Step ff1 and ff2 respectively, and represent embodiments and/or aspects of this process for preparing Compound X*. Another embodiment of this process for preparing Compound X* is a process for preparing Compound 9, which comprises Step hh as described above; and which further comprises:

(ff1) treating Compound 7: 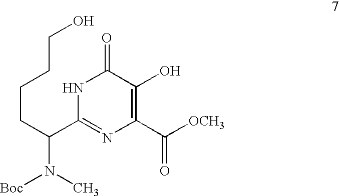
with CH₃SO₂X, wherein X is halogen, in the presence of a base to form a product which is (i) Compound 8, (ii) Compound 8a: 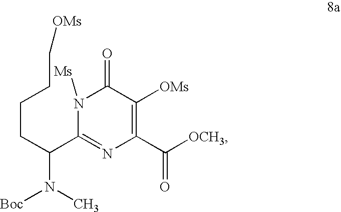
or (iii) a mixture of Compound 8 and Compound 8a;

(ff2) then:

(1) when the product is (i) Compound 8, proceeding directly to Step hh;

(2) when the product is (ii) Compound 8a, contacting the product with (a) a primary or secondary amine or (b) an alcohol, water, or an alcohol-water mixture in the presence of a base, to form Compound 8; or

(3) when the product is (iii) a mixture of Compounds 8 and 8a, optionally contacting the product with (a) a primary or secondary amine or (b) an alcohol, water, or an alcohol-water mixture in the presence of a base, to form additional Compound 8.

The present invention also includes a process for preparing a compound of Formula XI* which comprises Step hh-1 as described above; and which further comprises:

(ff1-1) reacting a compound of Formula VIII* with T-CH₂NH₂ to obtain a compound of Formula VII-1*:

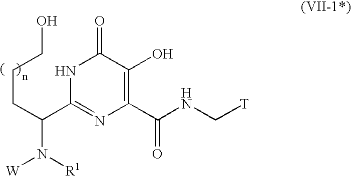

(ff1-2) treating a compound of Formula VII-1* with R^{I}SO₂X, wherein X is halogen, in the presence of a base to form a product which is (i) a compound of Formula VIII-1*, (ii) a compound of Formula VIII-2*, (iii) a compound of Formula VIII-3*, (iv) a compound of Formula VIII-1a*, or (v) a mixture of two to four components selected from the group consisting of Compounds VIII-1*, VIII-2*, VIII-3* and VIII-1a*;

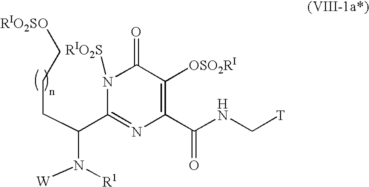

(ff2-1) then:

(1) when the product is (i) Compound VIII-1*, (ii) Compound VIII-2*, (iii) Compound VIII-3*, or a mixture thereof, proceeding directly to Step hh-1;

(2) when the product is (iv) Compound VIII-1a*, contacting the product with (a) a primary or secondary amine or (b) an alcohol, water, or an alcohol-water mixture in the presence of a base, to form Compound VIII-1*; or

(3) when the product is the mixture (v) containing Compound VIII-1a*, optionally contacting the product with (a) a primary or secondary amine or (b) an alcohol, water, or an alcohol-water mixture in the presence of a base, to form additional Compound VIII-1*.

The reaction conditions, bases, solvents, relative proportions of reactants and reagents, procedures, etc. described above as suitable with respect to Steps F1-1, F1-2 and F2-1 are suitable and applicable here to Step ff1-1, ff1-2 and ff2-1 respectively, and represent embodiments and/or aspects of this process for preparing Compound XI*. Another embodiment of this process for preparing Compound X* is a process for preparing Compound 10, which comprises Step hh-1 as described above; and which further comprises:

(ff1-1) reacting Compound 7 with 4-fluorobenzylamine to obtain Compound 7-1:

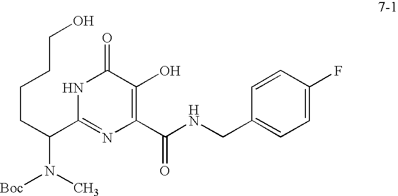

(ff1) treating Compound 7-1 with CH₃SO₂X, wherein X is halogen, in the presence of a base to form a product which is (i) Compound 8-1, (ii) Compound 8-2, (iii) Compound 8-3, (iv) Compound 8-1a, or (v) a mixture of two to four components selected from the group consisting of Compounds 8-1, 8-2, 8-3 and 8-1a;

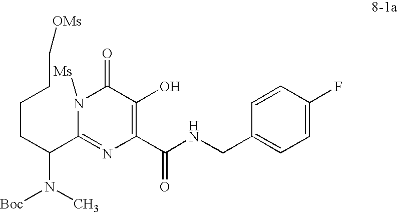

(ff2-1) then:

(1) when the product is (i) Compound 8-1, (ii) Compound 8-2, (iii) Compound 8-3 or a mixture thereof, proceeding directly to Step hh-1;

(2) when the product is (iv) Compound 8-1a, contacting the product with (a) a primary or secondary amine or (b) an alcohol, water, or an alcohol-water mixture in the presence of a base, to form Compound 8-1; or

(3) when the product is the mixture (v) containing Compound 8-1a, optionally contacting the product with (a) a primary or secondary amine or (b) an alcohol, water, or an alcohol-water mixture in the presence of a base, to form additional Compound 8-1.

The present invention also includes a process for preparing a compound of Formula X* which comprises Steps ff1, ff2, and hh as described above; and which further comprises:

(ee) heating (i) a mixture of compounds of Formula VIa* and VIb* or (ii) a compound of Formula VIc*: 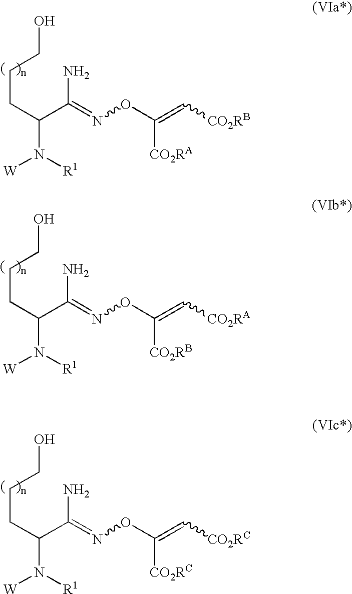
to obtain Compound VII*. The present invention also includes a process for preparing a compound of Formula XI* which comprises Steps ff1-1, ff1-2, ff2-1, and hh-1 as described above; and which further comprises Step ee as described above. The reaction conditions, bases, solvents, relative proportions of reactants and reagents, procedures, etc. described above as suitable with respect to Step E are suitable and applicable here to Step ee, and represent embodiments and/or aspects of this process for preparing Compound X*.

Another embodiment of the process for preparing Compound X* is a process for preparing Compound 9, which comprises Step ff1, ff2, and hh as described above; and which further comprises:

(ee) heating Compound 6: 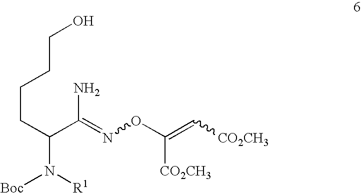
to obtain Compound 7. Another embodiment of the process for preparing Compound XI* is a process for preparing Compound 9, which comprises Step ff1-1, ff1-2, ff2-1, and hh-1 as described above; and which further comprises Step ee as described immediately above.

An aspect of the preceding embodiment for preparing Compound 9 is the process which comprises Steps ee, ff1, ff2, and hh as just described; and which further comprises:

(dd) reacting Compound 5: 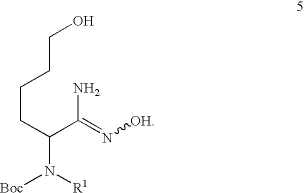
with dimethyl acetylene dicarboxylate to obtain Compound 6; and

which optionally further comprises:

(cc) reacting hydroxylamine or an acid salt thereof with Compound 4: 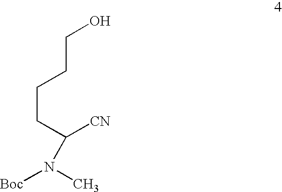
to obtain Compound 5; and

which optionally further comprises:

(aa) treating cyclic ether 1: 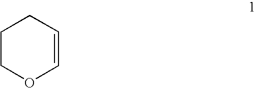
with an aqueous solution of a protonic acid to form an aqueous product mixture comprising Compound 2: 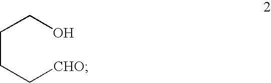
neutralizing the aqueous product mixture and then contacting the neutralized product mixture with methylamine, or an acid salt thereof, and an alkali metal cyanide to obtain Compound 3:

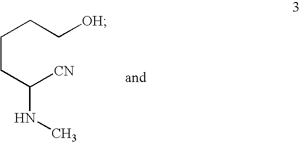

(bb) treating Compound 3 with (Boc)₂O or a Boc-halide to obtain Compound 4.

An aspect of the preceding embodiment for preparing Compound 9 is the process which comprises Steps ee, ff1-1, ff1-2, ff2-1, and hh-1 as just described; and which further comprises Step dd, optionally further comprises Step cc, and optionally further comprises Steps aa and bb.

The reaction conditions, bases, solvents, relative proportions of reactants and reagents, procedures, etc. described above as suitable with respect to Steps A, B, C, and D are suitable and applicable here to Steps aa, bb, cc, and dd respectively, and represent embodiments and/or aspects of this process for preparing Compound 2. In analogy with Step A, it is of course understood that Step aa of the process of the invention includes the case where the aqueous product mixture comprises Compound 2 alone or in a mixture with Compound 2a:

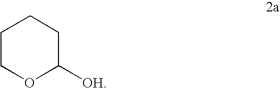

The present invention also includes a process for preparing Compound 11: 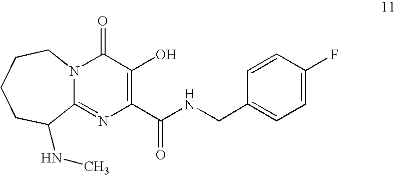
which comprises conducting Step hh as described above, and which further comprises:

(ii) reacting 4-fluorobenzylamine with Compound 2 to obtain Compound 10:

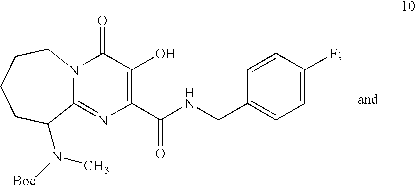

(jj) treating Compound 10 with a Boc cleaving agent to obtain Compound 11. The present invention also includes a process for preparing Compound 11, which comprises conducting Step hh-1 as described above, and which further comprises conducting Step jj. The reaction conditions, bases, solvents, relative proportions of reactants and reagents, procedures, etc. described above as suitable with respect to Steps I and J are suitable and applicable here to Steps ii and jj respectively, and represent embodiments and/or aspects of this process for preparing Compound 11.

Embodiments of the process for preparing Compound 11 include the process as described above and further comprising one or more of the pre-steps described above for preparing Compound 2.

The present invention also includes a process for preparing Compound 14: 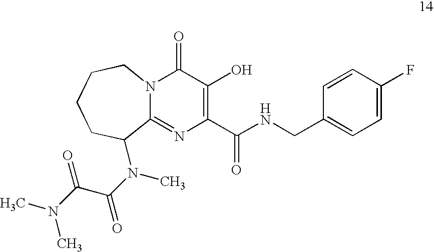
which comprises conducting (i) Step hh, Step ii, and Step jj, or (ii) Step hh-1 and Step jj as described above to obtain Compound 11; and which further comprises:

(11) either (i) reacting Compound 11 with (i) (CH₃)₂N—C(═O)—C(═O)—OC(═O)—O—C₁₋₄ alkyl, or (ii) reacting Compound 11 with C₁₋₄ alkyl-O—C(═O)—C(═O)-halide and then with (CH₃)₂NH, to obtain Compound 14.

Embodiments of the process for preparing Compound 14 include the process as described above and further comprising one or more of the pre-steps described above for preparing Compound 2.

Other embodiments of the present invention include any and all of the processes as originally defined and described above and any embodiments or aspects thereof as heretofore defined, further comprising isolating (which may be alternatively referred to as recovering) the compound of interest (including but not limited to any of the compounds of Formula In to XIV or any of the compounds 4, 5, 6, 7, 7-1, 8, 8-1, 8-2, 8-3, 8-1a, 9, 10, 11 or 14) from the reaction medium.

The progress of any reaction step set forth herein can be followed by monitoring the disappearance of a reactant (e.g., Compound VIII in Step H or Compound VIII-1 and/or Compound VIII-2 and/or Compound VIII-3 in Step H-1) and/or the appearance of the desired product (e.g., Compound X in Step H or Compound XI in Step H-1) using such analytical techniques as TLC, HPLC, IR, NMR or GC.

As is clear from the foregoing description, compounds embraced by Formula X or XI and precursors thereof are useful as intermediates in the preparation of Compounds XII, XIII and XIV, which are HIV integrase inhibitors useful, inter alia, in treating HIV infection. More particularly, carboxamide compounds representative of the compounds embraced by Formulas XII, XIII and XIV (e.g., Compound 14) have exhibited activity in an assay described in WO 02/30930 for inhibition of strand transfer in HIV integrase. Representative compounds have also exhibited activity in an assay (disclosed in Vacca et al., Proc. Natl. Acad. Sci. USA 1994, 91: 4096) for inhibition of acute HIV infection of T-lymphoid cells.

The term “hydrocarbyl” as used herein refers to a group (e.g., a C₁₋₂₀ hydrocarbyl group) consisting of carbon and hydrogen atoms and having a carbon atom directly attached to the rest of the molecule. Examples of hydrocarbyl groups include aliyl, alkenyl, alicyclic, saturated bicyclic, alkyl substituted alicyclic, aromatic, and alkyl substituted aromatic. The hydrocarbyl group is optionally substituted with one or more non-hydrocarbon substituents (e.g., oxo, halo, nitro, cyano, and alkoxy) and also optionally has one or more of its carbon atoms replaced with a heteroatom (e.g., N, O, or S) provided that the substituted hydrocarbyl group is not chemically reactive under the reaction/treatment conditions employed (e.g., in Step F1, the groups do not interfere or compete with the conversion of the OH groups in Compound VII to O-L groups) and do not interfere with subsequent reaction steps (e.g., Steps F2, optional G, and H).

The term “alkyl” refers to any linear or branched chain alkyl group having a number of carbon atoms in the specified range. Thus, for example, “C₁₋₆ alkyl” (or “C₁-C₆ alkyl”) refers to all of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms has been replaced with a halogen (i.e., F, Cl, Br and/or I). Thus, for example, “C₁₋₆ haloalkyl” (or “C₁-C₆ haloalkyl”) refers to a C₁ to C₆ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.).

The term “-alkylene-” refers to any linear or branched chain alkylene (or alternatively “alkanediyl”) having a number of carbon atoms in the specified range. Thus, for example, “—C₁₋₄ alkylene-” refers to the C₁ to C₄ linear or branched alkylenes. A class of alkylenes of particular interest with respect to the invention is —(CH₂)₁₋₄—, and sub-classes of particular interest include —CH₂)₁₋₄—, —CH₂)₁₋₃—, —(CH₂)₁₋₂—, and —CH₂—. Also of interest is the alkylene CH(CH₃)—.

The term “cycloalkyl” refers to any cyclic ring of an alkane having a number of carbon atoms in the specified range. Thus, for example, “C₃₋₈ cycloalkyl” (Or “C₃-C₈ cycloalkyl”) refers to cyclopropyl, cyclobutyl, cyclopentyl, cyclohexyl, cycloheptyl, and cyclooctyl.

The term “C₄₋₇ azacycloalkyl” (or “C₄-C7 azacycloalkyl”) means a saturated cyclic ring consisting of one nitrogen and from four to seven carbon atoms (i.e., pyrrolidinyl, piperidinyl, azepanyl, or octahydroazocinyl).

Unless expressly stated to the contrary, all ranges cited herein (i.e., process ranges such as a temperature range and ranges defined in the compounds set forth herein) are inclusive; i.e., the range includes the values for the upper and lower limits of the range as well as all values in between. Thus, for example, a heterocyclic ring described as containing from “1 to 4 heteroatoms” means the ring can contain 1, 2, 3 or 4 heteroatoms. It is also to be understood that any range (e.g., a temperature range) cited herein includes within its scope all of the sub-ranges within that range.

When any variable (e.g., R⁴ and R⁵) occurs more than one time in any constituent or in Formula I or Formula II or in any other formula depicting and describing compounds employed or included in the invention, its definition on each occurrence is independent of its definition at every other occurrence. Also, combinations of substituents and/or variables are permissible only if such combinations result in stable compounds.

The term “substituted” (e.g., as in “the aryl is optionally substituted with from 1 to 5 substituents . . . ”) includes mono- and poly-substitution by a named substituent to the extent such single and multiple substitution (including multiple substitution at the same site) is chemically allowed. Unless expressly stated to the contrary, substitution by a named substituent is permitted on any atom in a ring provided such ring substitution is chemically allowed and results in a stable compound.

Any heterocyclic ring substituent defined herein (e.g., HetA and HetB) can be attached to the rest of the compound via either a ring carbon atom or a ring heteroatom, provided such attachment is chemically allowed and results in a stable compound.

The term “solvent” in reference to any of the solvents employed in a reaction or treatment step set forth herein (e.g., solvent H in Step H) refers to any organic substance which under the reaction conditions employed in the step of interest is in the liquid phase, is chemically inert, and will dissolve, suspend, and/or disperse the reactants and any reagents so as to bring the reactants and reagents into contact and to permit the reaction to proceed.

The term “aging” and variants thereof (e.g., “aged”) mean allowing the reactants in a given reaction or treatment step to stay in contact for a time and under conditions effective for achieving the desired degree of conversion. The terms “aging” and variants thereof (e.g., “aged” are used herein interchangeably with the expression “maintaining at reaction temperature until the desired degree of conversion is achieved” and variants thereof (e.g., “maintained . . . ”) The term “catalytic amount” refers herein to any amount that allows the reaction of interest (e.g., acid treatment in Step A) to proceed under less extreme conditions (e.g., at a lower reaction temperature) and/or in a shorter reaction time compared to the reaction conditions and/or reaction time in the absence of the catalyst. A catalytic amount of a reagent can suitably be a substoichiometric amount of the reagent relative to the reactant substrate, such as an amount in a range of from about 0.001 to less than 1 mole (e.g., from about 0.005 to about 0.5 mole) per mole of the substrate.

The “squiggly” line in a structure (i.e., “ [symbol] ”) refers to a bond that attaches a group to a double bond and further denotes that that group is either in a cis configuration or a trans configuration with a group attached to the other end of the double bond. For example the “ [symbol] ” bond that attaches a CO₂R^{C} group to a carbon-carbon double bond in Compound VIc denotes that the CO₂R^{C} group is either in the cis configuration or the trans configuration with the CO₂R^{C} attached to the other end of the double bond. It is to be understood that a structural formula of a compound containing “ [symbol] ” bonds encompasses all isomeric forms of the compounds, singly and in mixtures.

An asterisk (“*”) in front of an open bond in the structural formula of a group marks the point of attachment of the group to the rest of the molecule.

10-camphorsulfonyl is 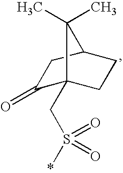
wherein the asterisk (*) indicates the point of attachment.

The term “% enantiomeric excess” (abbreviated “ee”) means the % major enantiomer less the % minor enantiomer. Thus, a 70% enantiomeric excess corresponds to formation of 85% of one enantiomer and 15% of the other.

Abbreviations used in the instant specification include the following:
Ac=acetyl
Alloc or ALLOC=allyloxycarbonyl
Bn=benzyl
Bz=benzoyl
Boc or BOC=t-butyloxycarbonyl
t-Bu=tertiary butyl
Cbz or CBZ=carbobenzoxy (alternatively, benzyloxycarbonyl)
DABCO=1,4-diazabicyclo[2.2.2]octane
DBN=1,5-diazabicyclo[4.3.0]non-5-ene
DBU=1,8-diazabicyclo[5.4.0]undec-7-ene
DEAD=diethylazodicarboxylate
DIAD=diisopropylazodicarboxylate
DIPEA=N,N′-diisopropylethylamine
DMAC=N,N-dimethylacetamnide
DMAD=dimethylacetylenedicarboxylate
DMF=N,N-dimethylformamide
EtOAc=ethyl acetate
EtOH=ethanol
h=hour(s)
IPA=isopropyl alcohol
IPAc=isopropyl acetate
KF=Karl Fisher titration for water
Me=methyl
Ms=mesyl (methanesulfonyl)
M-TBE=methyl tert-butyl ether
NMM=N-methylmorpholine
NMR=nuclear magnetic resonance
TEA=triethylamine
THF=tetrahydrofuran

The following example serves only to illustrate the invention and its practice. The example is not to be construed as limitations on the scope or spirit of the invention.

### EXAMPLE 1

### Step 1: Preparation of ω-Hydroxy N-Methyl aminonitrile 3

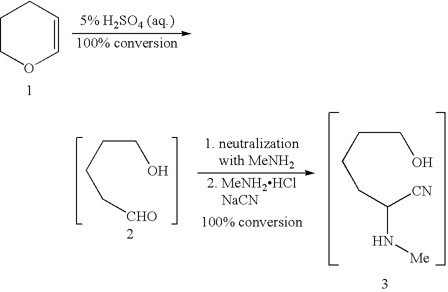

To a 5% H₂SO₄ aqueous solution (60 mL) was added 3,4-dihydro-2H-pyran (DHP; 21.1 g, 22.93 mL) at 20-35° C. The resulting solution was aged at 20-35° C. for 1 h. The reaction mixture was cooled to 0-5° C., and neutralized to pH=6-7 by 40% aqueous methylamine (5.3 mL). Methylamine hydrochloride (84.4 g) and sodium cyanide (12.25 g) were added respectively to the reaction mixture. The resulting solution was aged at room temperature for 36 h. The reaction mixture was extracted by IPAc (6×150 mL). The combined organic layers were concentrated to a total volume about 150 mL (assay yield about 91%) and was used in the next step. ¹H NMR (CDCl₃, 400 MHz) δ:3.81 (m, 1 H), 3.45 (m, 2H), 2.47 (s, 3H), 1.90-1.40 (m, 6H).

### Step 2: Preparation of co-Hydroxy N-Methyl N-Boc-aminonitrile 4

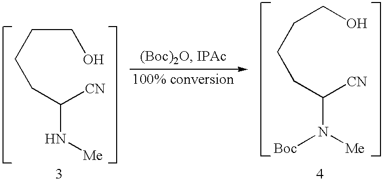

To a solution of co-hydroxy N-methyl aminonitrile 3 (0.2106 moles, 29.95 g) in IPAc (from Step 1) was added (Boc)₂O (48.3 g) at room temperature. The resulting solution was aged at 30-35° C. for 2 h (100% conversion by ¹H NMR). The reaction mixture was cooled to 0-5° C. and 5% NH₂OH/10% NH₄Cl (35 mL) was added. The resulting mixture was aged at 10-20° C. for 3 h. After a phase cut, the aqueous layer was extracted with IPAc (80 mL), the combined organic layers were washed with brine (50 mL), and then concentrated and solvent-switched to IPA (total volume 230 mL), which was used for next step. ¹H NMR (CDCl₃, 400 MHz) δ:5.18 (m, 1H), 3.64 (q, J=5.7 Hz, 2H), 2.88 (s, 3H), 1.88-1.75 (m, 3H). 1.65-1.61 (m, 2H), 1.49-1.46 (m, 1H), 1.18 (s, 9H).

### Step 3: Preparation of Hydroxyamidine 5

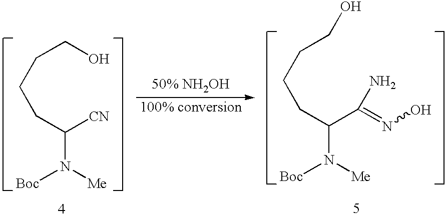

To a solution of N-Boc-aminonitrile 4 (0.2106 moles, 51.03 g) in IPA (total volume 230 mL) was added 50% hydroxylamine (16.2 mL) at ambient temperature. The resulting solution was aged at 60° C. for 3 h. The reaction mixture was then concentrated and solvent-switched to methanol solution (total volume 230 mL), which was used in the next step. ¹H NMR (CDCl₃, 400 MHz) δ:7.53 (br s, 1H), 4.84 (br s, 2H), 4.64 (t, J=7.1 Hz, 1H), 3.71-3.62 (m, 2 H), 2.72 (s, 3H), 1H), 1.76 (1.55 (m, 3H), 1.49 (s, 9H), 1.42-1.23 (m, 2H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time: amidoxime-6.152 minutes and 6.256 minutes (two isomers)

### Step 4: Preparation of O-Alkene Amidoxime 6

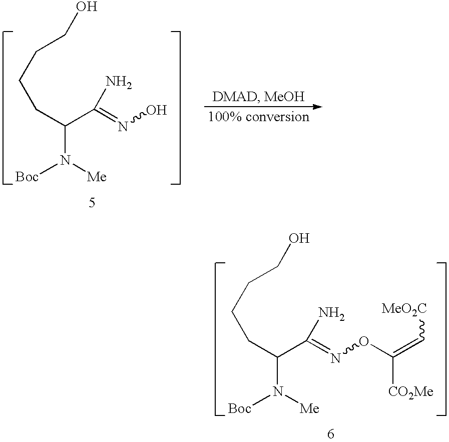

To a solution of hydroxyamidine 5 (about 0.2106 mole, 57.93 g) in methanol (total volume 230 mL) was added dimethyl acetylenedicarboxylate (27.10 mL) at room temperature. The resulting solution was aged at room temperature for 16 h. The reaction mixture was concentrated and solvent-switched to cumene at 40-60° C. (total volume 430 mL). The solution was used in the next step. ¹H NMR (CDCl₃, 400 MHz) δo: 5.82 (s, 0.28H), 5.73 (s, 0.72H), 5.44 (br s, 1.77H), 5.25 (br s, 0.56H), 4.61 (m, 1H), 3.89 (s, 0.84H), 3.84 (s, 2.16H), 3.72 (s, 2.16 H), 3.68 (s, 0.84H), 3.65-3.58 (m, 2H), 2.73 (s, 0.84H), 2.71 (s, 2.16H), 1.90-1.52 (m, 4H), 1.47 (s, 9H), 1.43-1.30 (m, 2H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time: amidoxime 6-12.051 mlinutes, 12.315 minutes, ratio ca 3.6:1.

### Step 5: Preparation of Pyrimidine 7

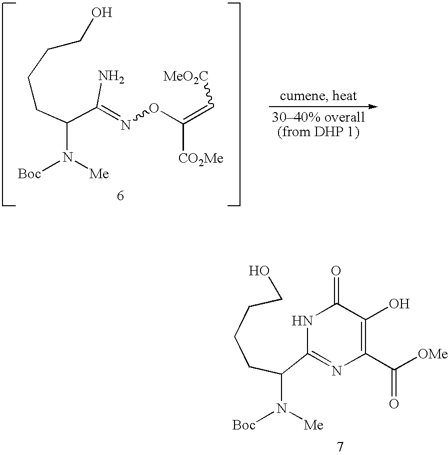

A solution of O-alkene amidoxime 6 (about 0.2106 moles, 87.91 g) in cumene (total volume 430 mL) was heated at 120° C. (inside temperature) for 12 h. The reaction mixture was then cooled to about 60° C., concentrated to a total volume 250 mL, then diluted with EtOAc (250 mL), and cooled to 25-35° C. 5% Sodium bicarbonate (330 mL, about 1 equiv.) was then slowly added, and the resulting solution was aged at 25-35° C. for 0.5 h. After a phase cut, the organic layer was extracted with 5% sodium bicarbonate (180 mL) again. The combined aqueous extracts were acidified by 5 N HCl to pH=2-3, and extracted by EtOAc (3×250 mL). The combined organic layers were washed with brine (150 mL). The organic solution was concentrated and solvent-switched to THP (about 30-40% yield overall, KF about 100-150 ppm). ¹H NMR (CDCl₃, 400 MHz) δ:10.66 (br s, 2H), 4.77 (m, 1H), 4.01 (s, 3H), 3.72-3.67 (m, 2H), 2.77 (s, 3H), 2.20-1.55 (m, 5H), 1.48 (s, 9H), 1.43-1.35 (m, 1H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time: pyrimidine 7-9.905 minutes.

Step 6: Preparation of Bismesyl-Pyrimidine 8

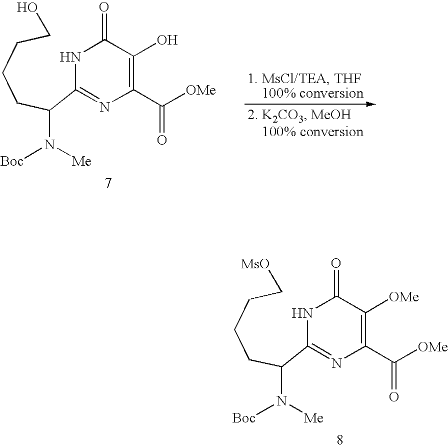

To a solution of pyrimidine 7 (43.5 g, about 80% pure, 0.09029 moles) in THF (275 m L) was slowly added TEA (37.8 mL) and MsCl (21.0 mL) at the same time at 0-5° C. over 1 h. The resulting solution was aged at the same temperature for 4 h. The solid was filtered off, washed with THF (3×100 mL). The combined filtrations were concentrated and solvent-switched to methanol (total volume 200 mL). To the trimesyl-pyrimidine in methanol solution was added potassium carbonate (12.5 g, 0.09029 moles) at 10-20° C. The resulting solution was aged at the same temperature for 6-10 h (monitored by HPLC). The reaction mixture was neutralized to pH=6-7 by 5 N HCl, and concentrated to a total volume about 100 mL. 16% brine (100 mL) was added, and the resulting solution was extracted by EtOAc (3×100 mL). The combined organic layers were washed with brine (50 mL), concentrated and solvent-switched to DMF. The by-product (MeSO₃Me), which was generated in 1 equiv from the selectively hydrolysis of the trimesyl-pyrimidine, was removed by azeotrope with DMF at 60-65° C. (monitored by ¹H NMR until<10 mole %). The concentration of bismesyl-pyrimidine 8 in DMF was about 0.3 M (total volume 300 mL). ¹H NMR (CDCl₃, 400 MHz) δ:11.00 (br s, 1H), 4.78 (d, J=7.8 Hz, 1H), 4.24-4.15 (m, 2H), 3.95 (s, 3H), 3.50 (s, 3H), 2.99 (s, 3H), 2.81 (s, 3H), 2.12-2.11 (m, 1H), 1.90-1.76 (m, 2H), 1.46 (s, 9H), 1.43-1.35 (m, 2H).

HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold 20 for 5 min, 10:90 to 90:10 (A)/(B) over 10 seconds; Flow Rate: 1 mL/min. Retention time: trimesyl-pyrimidine-14.140 minutes; bismesyl-pyrimidine-12.760 minutes.

Step 7: Preparation of Seven-Membered Ring-Pyrimidine Mesylate 9

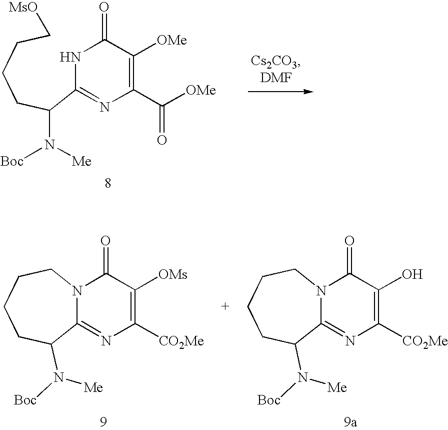

To a solution of bismesyl-pyrimidine 8 (0.09029 moles, 48.90 g) in DMF (total volume 300 mL) was added cesium carbonate (35.30 g) at room temperature. The resulting slurry was aged at 55° C. for 2-3 h (76% conversion by HPLC). After being neutralized to pH=7, the reaction mixture was diluted with 250 mL of water, extracted with IPAc (2×250 mL). The combined organic layers were washed with brine (2×200 mL). The organic layer was concentrated to give crude product. Half of the crude product was purified by passing a short column (silica gel, hexane: EtOAc 2:1) to afford desired product 9 (6.00 g, 98 A % pure), and 9a (2.3 g, 40 A % pure). The overall yield from DHP to cyclized product is about 13% after correction. ¹H NMR (CDCl₃, 400 MHz) For compound 9: δ:5.34 (m, 1H), 5.22 (m, 1H), 3.93 (s, 3H), 3.51 (s, 3H), 3.47 (m, 1H), 2.97 (s, 3H), 2.20-2.05 (m, 3H), 1.90-1.65 (m, 2H), 1.44 (s, 9H), 1.24 (m, 1H). For compound 9a: 11.86 (br s, 1H), 7.90-7.55 (br s, 1H), 7.31 (dd, J=8.5, 5.4 Hz, 2H), 7.06 (t, J=8.5 Hz, 2H), 5.404.90 (m, 2H), 4.534.40 (m, 2H), 3.45-3.23 (m, 1H), 2.23-2.05 (m, 3H), 1.86-1.76 (m, 1H), 1.74-1.64 (m, 1H), 1.47-1.37 (m, 1H), 1.30 (s, 9H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time: the seven-membered ring-pyrimidine mesylate 9: 13.969 minutes; the seven-membered ring-pyrimnidine 9a: 13.141 minutes.

Alternative procedure using LiH was also employed: To a solution of bismesyl-pyrimidine 8 (65 mg) in dioxane (1 mL) was added LiH powder at room temperature. The resulting mixture was aged at 65° C. for 4 h. The reaction mixture was then cooled to room temperature and 1 N HCl was added to quenched the excess LiH. The solution was extracted with EtOAc (2×5 mL). The combined organic layer was washed with brine, and then concentrated. The residue was purified by flash chromatography (silica gel, hexane:EtOAc=2:1) to afford seven-membered ring-pyrimidine mesylate 9 (45.6 mg, 85%). ¹H NMR (CDCl₃, 400 MH) δ:5.34 (m, 1H), 5.22 (m, 1H), 3.93 (s, 3H), 3.51 (s, 3H), 3.47 (m, 1H), 2.97 (s, 3H), 2.20-2.05 (m, 3H), 1.90-1.65 (m, 2H), 1.44 (s, 9H), 1.24 (m, 1H).

### Step 8: Preparation of Seven-Membered Ring-Pyrimidine Amide 10

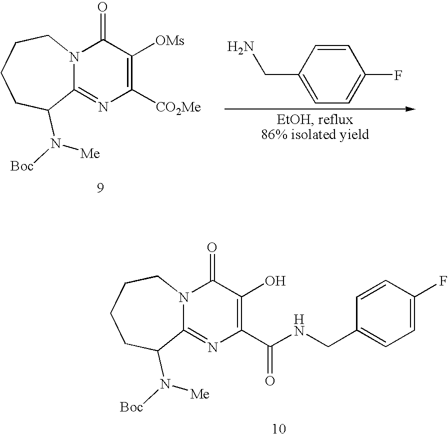

To a solution of seven-membered ring-pyrimidine mesylate 9 (6 g, 0.01347 moles) in EtOH (80 mL) was added 4-fluorobenzylamine (5.060 g, 0.04041 moles). The resulting solution was reflux for 8 h. (100% conversion by HPLC). The reaction mixture was concentrated to about 20 mL total volume, and 80 mL of EtOAc was added. To the resulting solution was added 20% brine (15 mL), 4 N HCl (15 mL), and water 10 mL). After a phase cut, the aqueous layer was back-extracted with EtOAc (25 mL). The combined organic layers were washed with 4 N HCl: 20% brine (1:1, 3×15 mL), brine (15 mL). The organic solution was concentrated to a total volume about 30 mL. Hexane (70 mL) was slowly added to the solution over 1 h. The resulting slurry was aged at 0-5° C. for 1 h. The crystalline solid was filtered off, washed with hexane:EtOAc (4:1, 50 mL), dried under vacuum with nitrogen sweep to afford seven-membered ring-pyrimidine amide 10 (5.30 g, 86%, HPLC>97 A %). ¹H NMR (CDCl₃, 400 MHz) δ:11.85 (br s, 1H), 7.84 (br s, 0.5H), 7.68 (br s, 0.5H), 7.31 (m, 2H), 7.04 (m, 2H), 5.40-4.90 (m, 2H), 4.53 (m, 2H), 3.38 (m, 1H), 2.87 (s, 3H), 2.20-2.15 (m, 3H), 1.90-1.40 (m, 3H), 1.37 (s, 9H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time: the seven-membered ring-pyrinmidine 10-15.467 minutes.

### Step 9: Preparation of Seven-Membered Ring-Pyrimidine Amide Hydrochloride Salt 11

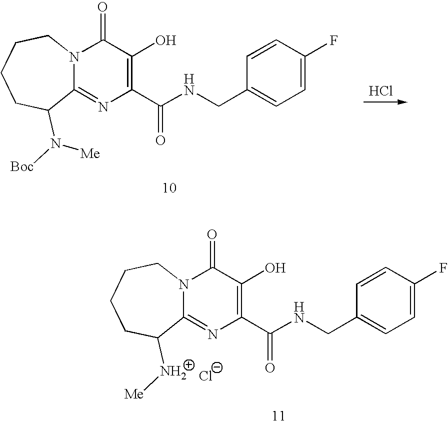

To a solution of ethyl acetate (3.5 mL) was bubbled HCl gas (0.5389 g, 0.01478 moles), at −30 to −20° C. N-Boc-seven-membered ring pyrimidine amide 10 (crystalline solid, 0.8500 g, 0.001846 moles) was charged to the HCl-EtOAc solution at −30 to −20° C. The resulting solution was slowly warmed to room temperature over 2.5 h, and aged at room temperature for 0.5 h (100% conversion by HPLC). The reaction mixture was diluted by EtOAc (7 mL). The resulting slurry was aged at 0-5° C. for 1 h. The crystalline solid was filtered off, washed with EtOAc, hexane, dried under vacuum with nitrogen sweep to afford desired product 11 (98% isolated yield, >97 A % pure). ¹H NMR (CDCl₃, 400 MHz) δ:12.35 (s, 1H), 9.96 (t, J=6.3 Hz, 1H), 9.51 (br s, 1H), 9.19 (br s, 1H), 7.42 (dd, J=8.5, 5.6 hz, 2H), 7.19 (t, J=8.5 Hz, 2H), 4.92 (dd, J=14.5, 5.1 Hz, 1H), 4.71 (m, 1H), 4.57-4.45 (m, 2H), 3.52 (t, J=14.5 Hz), 2.65 (t, J=5.0 Hz, 3H), 2.30 (br d, J=12.6 Hz, 1H), 1.99-1.92 (m, 1H), 1.90-1.75 (m, 2H), 1.68-1.60 (m, 1H), 1.41-1.33 (m, 1H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time: the seven-membered ring-pyrimidine hydrochloride salt 11-8.118 minutes.

### Step 10: Preparation of Racemic N-(2-{[(4-fluorobenzyl)amino]carbonyl}-3-hydroxY4-oxo-4,6,7,8,9,10-hexahydropyrnidor[1,2-α]azepin-10-yl)-N,N′,N′-trimethylethanediamide 14

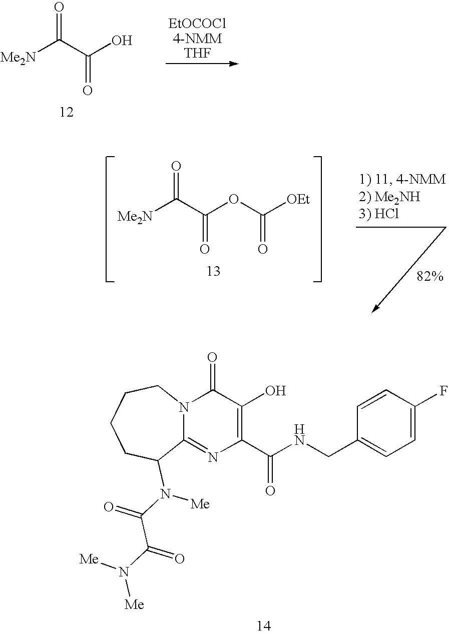

To a solution of acid 12 (96% pure, 122 mg, 1.000 mmole) in THF (3 mL) was added ethyl chloroformate (92 μl, 0.104 g, 0.960 mmoles) at 0-5° C. Then, 4-NMM (106 μl, 0.0971 g, 0.960 mmole) was slowly added to the reaction mixture at 0-5° C. The reaction mixture was aged at the same temperature for 2 h. The pyrimidine hydrochloride salt 11 (79.4 mg, 0.200 mmole) was added as a solid to the mixed-anhydride solution at 0-5° C., and aged at the same temperature for 5 h, and then at 5-10° C. for another 2 h (100% conversion by HPLC). Dimethylamine aqueous (40%, 158 μl, 0.141 g, 1.250 mmole) was added to the reaction mixture, and the mixture aged at 10-15° C. for 1 h, wherein the reaction was monitored by HLPC to assure complete conversion. The reaction mixture was acidified by 2 N HCl to adjust to pH=34 at 5-15° C. EtOAc (6 mL) and brine (2 mL) were added, respectively. After phase cut, the organic layer was washed with 1 N HCl (2 mL), brine (2×2 mL). The organic layer was concentrated to a total volume of 1 mL. Hexane (5 mL) was slowly added over 0.5 h. The resulting slurry was aged at 0-5° C. for 1 h. The crystalline solid was filtered off, washed with hexane/EtOAc (5: 1), MTBE, dried under vacuum with nitrogen sweep to give the title compound 14 (75.6 mg, 82%). ¹H NMR (CDCl₃, 400 MHz) δ:12.13 (s, 1H), 9.41 (br s, 1H), 7.38 (dd, J=8.5, 5.4 Hz, 2H), 7.00 (t, J=8.5 Hz, 2H), 5.40 (brs, 1H), 5.29 (dd, J=14.5, 6.0 Hz, 1H), 4.60 (dd, J=14.5, 6.6 Hz, 1H), 4.52 (dd, J=14.5, 6.3 Hz, 1H), 3.35 (dd, J=14.5, 11.6 Hz, 1H), 3.04 (s, 3H), 3.01 (s, 3H), 2.98 (s, 3H), 2.23-2.12 (m, 3H), 1.95-1.81 (m, 2H), 1.58-1.49 (m, 1H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time: the title compound 14-12.191 minutes.

### EXAMPLE 2

### Step 1: Preparation of ω-Hydroxy N-Methyl aminonitrile 3

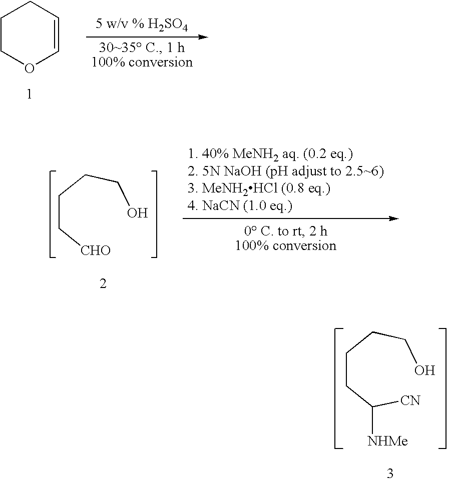

To a 5 w/v % H₂SO₄ aqueous solution (14.3 L) was added dropwise 3,4-dihydro-2H-pyran (5.000 kg) for 30 min at 30-35° C. The resulting solution was aged for 30 min at the same temperature. To the reaction mixture was added 40% aqueous methylamine (0.2 eq., 1.04 L) at 0-5° C., and the pH was adjusted to pH=3˜7 with SN aqueous NaOH (ca. 0.59 L). Methylamine hydrochloride (0.8 eq., 3.210 kg) was added to the reaction mixture and cooled to ⁰° C. In another vessel, sodium cyanide (1.0 eq., 2.913 kg) was dissolved in water (6.797 kg) to give aqueous NaCN (30 wt %) solution and cooled to 0° C. The reaction mixture was charged into aqueous NaCN solution for 1.5 hr (exothermic) at 0° C. The resulting solution was aged at rt for 2 h, and then the conversion was checked by ¹H NMR analysis (reaction mixture 0.1 mL+D₂O 0.5 mL: conversion 100%, 83-86% assay yield; sodium salicylate was used as internal standard)). The aqueous reaction mixture was washed with heptane (20 L) to remove side-products. The water layer was extracted by IPAc (4×35.8 L). The combined IPAc solution was concentrated to a total volume of about 50 L, which will be used for next step. ¹H NMR (CDCl₃, 400 MHz) δ:3.81 (m, 1H), 3.45 (m, 2H), 2.47 (s, 3H), 1.90-1.40 (m, 6H).

### Step 2: Preparation of ω-Hydroxy N-Methyl N-Boc-aminonitrile 4

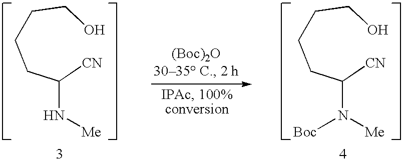

To a solution of cohydroxy N-methyl aminonitrile 3 (50.52 moles, 7.185 kg, based on 85% yield from 1 in IPAc (50 L, from last step) was added dropwise IPAc (5 L) solution of (Boc)₂O (1.05 eq., 53.05 moles, 15.58 kg) at 30-35° C. for 30 min. The resulting solution was aged at the same temperature for 1.5 h (conversion 100% by ¹H NMR). To the reaction mixture was added 4.5% NH₄OH/10% NH₄C_{l (}8.5 L; prepared by mixing 12.5 g of 28% aqueous NH₄OH, 7 g NH₄Cl, and 50.5 g water) at 20-25° C. The resulting mixture was aged at the same temperature over night. After a phase cut, the aqueous layer was extracted by IPAc (12 L). The combined organic layer was washed with 1N aqueous NaOH (3×20 L) at 0-5° C., 10% aqueous w/w NH₄Cl (12 L) and 20% w/w brine (12 L) at the same temperature. The yield of 4 was assayed by HPLC (10.70 kg, 74% from DHP 1. ¹H NMR (CDCl₃, 400 MHz) δ:5.18 (m, 1H), 3.64 (q, J=5.7 Hz, 2H), 2.88 (s, 3H), 1.65-1.61 (m, 2H), 1.49-1.46 (m, 1H), 1.18 (s, 9H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time for the Boc-amiine 4: 11.490 min.

### Step 3: Preparation of Hydroxyamidine 5

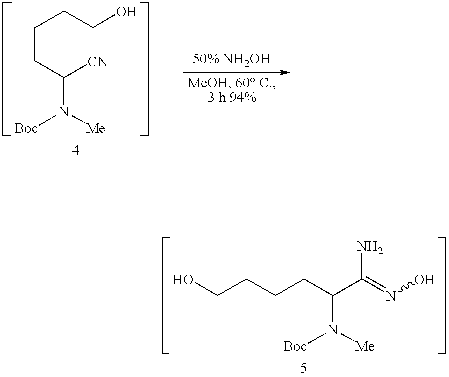

IPAc solution of N-Boc-N-methylaminonitrile 4 (10.70 kg assay, 44.16 mol) was concentrated and solvent-switched to methanol under reduced pressure at 20-35° C. Solvent composition was checked on GC to confirm IPAc is less than 1 v/v %. At this point, the total volume of the methanol solution was about 32 L. MeOH solution of 4 was warmed to 60° C., and 50% NH₂OH aqueous solution (2.84 L, 46.37 mol, 1.00 eq) was added at 60° C. for 3.0 hr for avoiding accumulation of NH₂OH. The amount of NH₂OH was carefully adjusted to exactly 1.00 eq (excess amount of NH₂OH would cause trouble in the following steps). The resulting solution was aged at a 60° C. for 3 h. The reaction was monitored by HPLC (conversion>98%, residual NH₂OH<1% (the sample was treated with DMAD and the amount of NH₂OH was assayed as DMAD adduct)). The yield of hydroxyamidine 5 was assayed by HPLC (11.43 kg, 94% from 4). The concentration was adjusted to about 0.20 kg of A/kg solution). ¹H NMR (CDCl₃, 400 MHz) δ:7.53 (br s, 1H), 4.84 (br s, 2H), 4.64 (t, J=7.1 Hz, 1H), 3.71-3.62 (m, 2H), 2.72 (s, 3H), 2.00 (br s, 1H), 1.92-1.82 (m, 1H), 1.76 (1.55 (m, 3H), 1.49 (s, 9H), 1.42-1.23 (m, 2H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time for the hydroxyamidine 5:6.152 min. and 6.256 min. (two isomer).

### Step 4: Preparation of DMAD Adduct 6

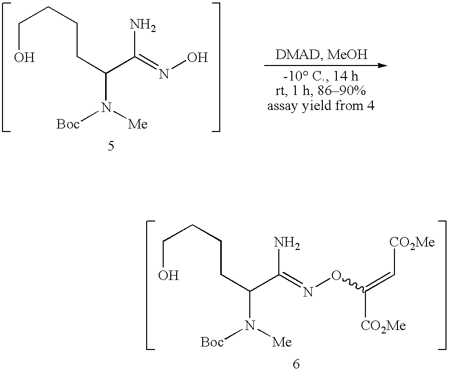

To a solution of hydroxyamidine 5 (23.86 kg) in methanol solution was added DMAD (1.05 eq., 11.19 L, 12.94 kg, 91.00 moles) at −15° C. to −5° C. The resulting solution was aged at the same temperature for 14 h, and then allowed to warm to room temperature (conversion>98 A % by HPLC). The reaction mixture was solvent switched to xylenes at 25-40° C. until methanol<5 mole % compared to DMAD adduct 6 (total volume 346 L). The assay yield is 86-90% from N-Boc-N-methylaminonitrile 4. The resulting solution was divided in half for next step (two batches). ¹H NMR (CDCl₃, 400 MHz) δ:5.82 (s, 0.28H), 5.73 (s, 0.72H), 5.44 (br s, 1.77H), 5.25 (br s, 0.56H), 4.61 (m, 1H), 3.89 (s, 0.84H), 3.84 (s, 2.16H), 3.72 (s, 2.16H), 3.68 (s, 0.84H), 3.65-3.58 (m, 2H), 2.73 (s, 0.84H), 2.71 (s, 2.16H), 1.90-1.52 (m, 4H), 1.47 (s, 9H), 1.43-1.30 (m, 2H). HPLC conditions: Column: Zorbax, Rx C8 250×4.6 mm; Temperature: 30° C.; Detection at 210 nm; Mobile Phase: 0.1% aq H₃PO₄ (A)tMeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time for the DMAD adduct 6: 12.051 min., 12.315 min., ratio ca 4.2:1.

### Step 5: Preparation of Pyrimidone 7

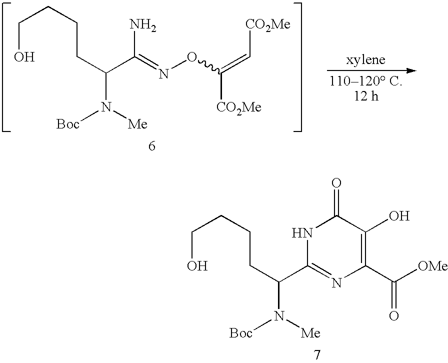

A solution of crude DMAD adduct 6 (calcd for 37.13 mol, 15.50 kg) in xylenes (total volume 173 L) was heated at 110-120° C. until consumption of desired DMAD adduct 6 (retention time 12.051 min, and undesired DMAD adduct 6 retention time 12.315 min). Typically, the reaction reached>98 A % conversion in 12-18 h. After the reaction was completed, the mixture was cooled to 50° C., and EtOAc (22.3 L) was added to the mixture. The resulting reaction mixture was extracted with 5% w/V NaHCO₃ aqueous (0.595M, 46.8 L, 0.75 eq) at 37° C. and (46.8 L, 0.75 eq) at room temperature. At this point, desired product 7 lost in organic layer was less than 2 wt %. To the combined aqueous solution was added EtOAc (59.4 L). To the resulting two-phase solution was slowly added 6 N HCl aqueous solution (9.8 L, 1.59 equiv.) to adjust the pH to 2.5-3.5. NaCl (9.28 kg) was added to the rmixture and the mixture was stirred at rt until NaCl dissolved (about 0.5 h). After a phase cut, the aqueous layer was extracted with EtOAc (16.6 L). At this point, desired product 7 lost in aqueous layer was less than 3 wt %. The combined organic layer was washed with sat. brine (11.2 L). The assay yield was 46% (7.72 kg of pyrimidone 2) overall from N-Boc-N-methylaminonitrile 4. The organic solution was concentrated and azeotroped with EtOAc until the KF was less than 600 ppm at a total volume of 28 L solution. The solution was inline filtered to remove some solid (NaCl). The resulting solution was concentrated and solvent switched to DMAc (total volume about 58 L), which was used in next step reaction. At this point, the remaining EtOAc in the DMAc solution and KF of the DMAc solution were less than 5 mole % compared to pyrimidone 7, and less than 230 ppm, respectively. ¹H NMR (CDCl₃, 400 MHz) δ:10.66 (br s, 21H), 4.77 (m, 1H), 4.01 (s, 3H), 3.72-3.67 (m, 2H), 2.77 (s, 3H), 2.20-1.55 (m, 5H), 1.48 (s, 9H), 1.43-1.35 (m, 1H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time for the pyrimidone 7:9.905 min.

### Step 6: Preparation of Bicyclic Pyrimidone 10

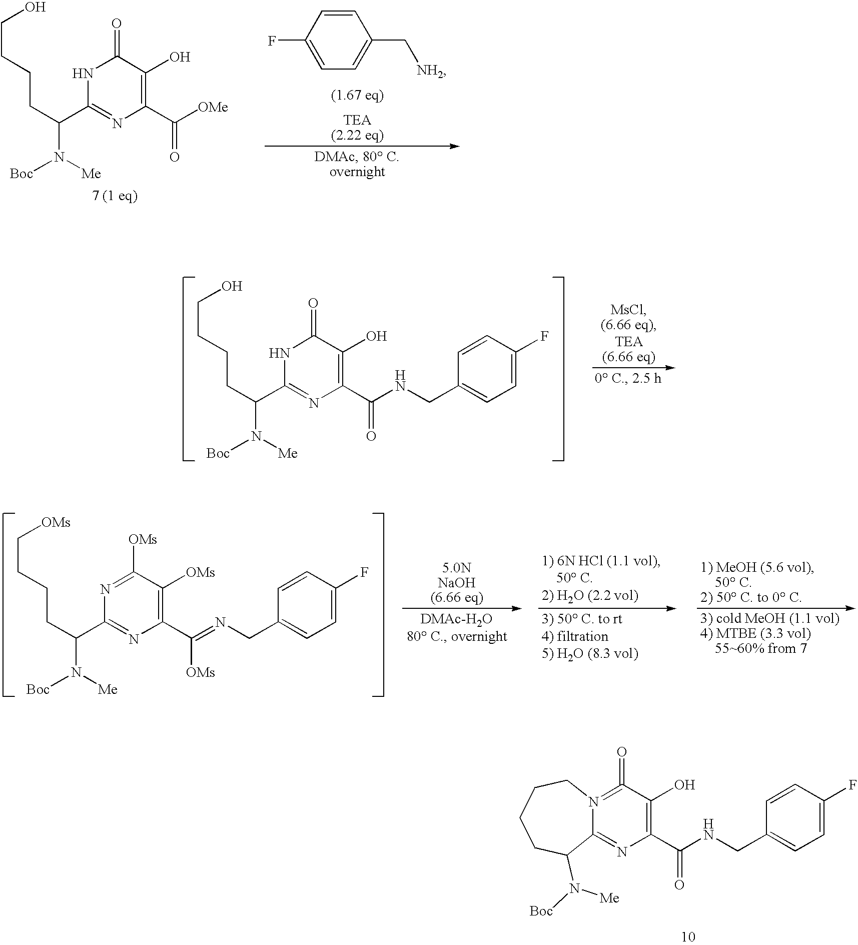

To a degassed solution of pyrimidone 7 in DMAc solution (5.04 kg of 7, 13.09 mol; total volume 37.9 L) was added Et₃N (2.94 kg, 29.05 mole, 2.22 eq.) and 4-fluorobenzylamine (2.73 kg 21.79 mol, 1.67 eq.) at rt, respectively. The resulting mixture was aged at 78-82° C. overnight. The reaction mixture was cooled to 0-2° C. To the solution was added Et₃N (8.82 kg, 87.15 mole, 6.66 eq.) in one portion at the same temperature. MsCl (9.98 kg, 87.15 mol, 6.66 eq.) was added dropwise below 10° C. (highly exothermic for this reaction). The resulting slurry was aged for 1 h at 0-2° C. Then, 5N aqueous NaOH (20.57 kg, 87.15 mol, 6.66 eq.) was added dropwise below 20° C. The mixture was warmed to 78-82° C., and aged for 24 h at 78-82° C., and then cooled to 50° C. 6N aqueous HCl (5.88 kg, 1.11 vol) was added dropwise over 1 hat 50° C. (pH was adjusted to 2.0-2.5). The crystalline product 10 was generated at pH about 5. The slurry was aged for 1 h at 50° C. H₂O (11.76 kg, 2.22 vol) was added dropwise over 1 h at the same temperature. The resulting slurry was stirred for 1 h at 50° C., cooled to 25° C. over 1-2 h, aged overnight (11 h) at 25° C. At this point, bicyclic pyrimidone 10 remaining in the supernatant was less than 1.3 wt %. The crude product 10 was collected by filtration, washed with cold (16° C.) H₂O (20.17 kg), rinsed with cold (16° C.) H₂O (20.17 kg), and dried under reduced pressure at 50° C. for 8 h. The blown crude product 10 was corrected in 7.50 kg with>90 A % purity.

The crude product 10 (7.50 kg) was then dissolved in methanol (25.2 kg) at 50° C. The resulting solution was aged for 1 h at the same temperature, and slowly cooled down to 20° C. over 2 h, and then aged for overnight (15 h) at 20° C. The resulting slurry was cooled down to 0° C. over 1-2 h, and aged for 1.5 h at the same temperature. At this point, bicyclic pyrimidone 10 remaining in the supernatant was less than 6.1 wt % by HPLC assay. The product was collected by filtration, washed with cold (0-5° C.) MeOH (5.40 kg) and MTBE (6.80 kg), rinsed with MTBE (3.30 kg), and dried under reduced pressure at 50° C. overnight. Thus, bicyclic pyrimidone 10 was corrected as a white crystalline solid (4.04 kg, 66% isolated yield from 7, >98.5 A % purity). ¹H NMR (CDCl₃, 400 MHz) δ:11.85 (br s, 1H), 7.84 (br s, 0.5H), 7.68 (br s, 0.5H), 7.31 (m, 2H), 7.04 (m, 2H), 5.40-4.90 (m, 2H), 4.53 (m, 2H), 3.38 (m, 1H), 2.87 (s, 3H), 2.20-2.15 (m, 3H), 1.90-1.40 (m, 3H), 1.37 (s, 9H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time for the bicyclic pyrimidone 10, 15.467 min.

### Step 7: Preparation De-Boc Amine Hydrochloride Salt 11

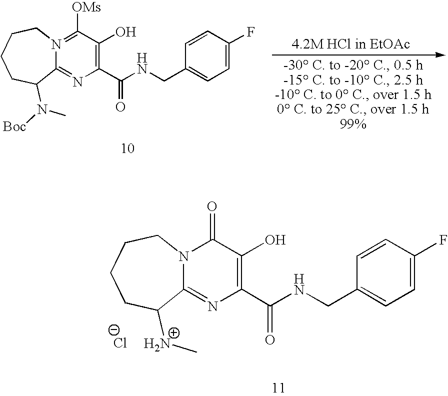

To a 100 L round bottom flask, equipped with an overhead stirrer, thermocouple, water-cooled condenser, and nitrogen inlet, was charged ethyl acetate (17.3 L). To the solution of ethyl acetate was bubbled HCl gas (3.269 Kg), at −30 to −20° C. Bicyclic pyrimidine 10 (crystalline solid, 4.129 kg, 8.976 mol) was slowly charged to the HCl-EtOAc solution at −30 to −20° C. The resulting solution was aged at −30 to −20° C. for 0.5 h, at −15 to −10° C. for 2 h, at −10 to 0° C. for 1.5 h, and slowly warmed to 25° C. over 1.5 h, then aged at 25° C. for 4 h (100% conversion by HPLC). To the reaction mixture was slowly added EtOAc (28.8 L) over 1 h at 25° C. The resulting slurry was aged at 25° C. for 4 h. The crystalline solid was filtered off, washed with EtOAc (8.3 L), heptane (8.3 L), dried under vacuum with nitrogen sweep to afford desired product 11 (3.584 kg, 99% isolated yield, 99.3 A % pure, 97.9 wt %). ¹H NMR (CDCl₃, 400 MHz) δ:12.35 (s, 1H), 9.96 (t, J=6.3 Hz, 1H), 9.51 (br s, 1H 7.42 (dd, J=8.5, 5.6 hz, 2H), 7.19 (t, J=8.5 Hz, 2H), 4.92 (dd, J=14.5, 5.1 Hz, 1H), 4.71 (m, 1H), 4.57-4.45 (m, 2H), 3.52 (t, J=14.5 Hz), 2.65 (t, J=5.0 Hz, 3H), 2.30 (br d, J=12.6 Hz, 1H), 1.99-1.92 (m, 1H), 1.90-1.75 (m, 2H), 1.68-1.60 (m, 1H), 1.41-1.33 (m, 1H). HPLC conditions: Column: Zorbax, Rx C8 250×4.6 mm; Temperature: 30° C.; Detection at 210 mn; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; flow rate: 1 mL/min. Retention time for the amine hydrochloride salt 11: 8.118 min.

### Step 8: Preparation of Free Amine 11a

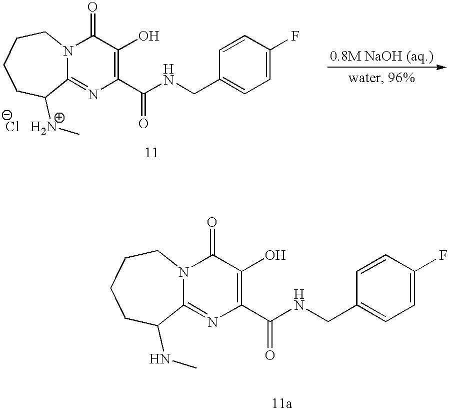

The amine HCl salt 11 (3.58 kg, 8.82 mol) was slurried in water (GMP, 26.25 L) in a 100 L three-neck round bottom flask equipped with nitrogen inlet, reflux condenser, thermocouple and overhead mechanical stirring. Sodium hydroxide (5.0 N, 1.76 L) was diluted with 8.75 L GMP water. The sodium hydroxide solution was added dropwise to the HCl salt slurry with an addition funnel over 2 h. The mixture was aged at room temperature overnight with vigorous stirring. After 24 h the supernatant is sampled and chloride analysis was undertaken to ensure complete conversion to the racemic free amine. The crystalline solid was filtered off, washed with 1×3.5 L of GMP water (slurry wash) followed by 2×3.5 L GMP water washes (displacement washes). The cake was then washed with 2×3.5 L of 1:1/MTBE: n-heptane and dried under vacuum with a nitrogen sweep to give free amine 11a (3.06 kg, 96%). ¹H NMR (CDCl₃, 400 MHz) δ:7.94 (br s, 1H), 7.33 (dd, J=8.4, 5.6 Hz, 2H), 7.06 (t, J=8.4 Hz, 2H), 5.03 (dd, J=14.1, 6.2 Hz, 1H), 4.77-4.54 (m, 2H), 3.89 (bt, J=10.2 Hz, 1H), 3.73 (d, J=10.2 Hz, 1H), 2.44 (s, 3H), 2.08-1.55 (m, 6H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time for the free amine 11a: 8.118 min.

### Step 9: Preparation of Chiral Amine-(L)-DTTA Salt 11b

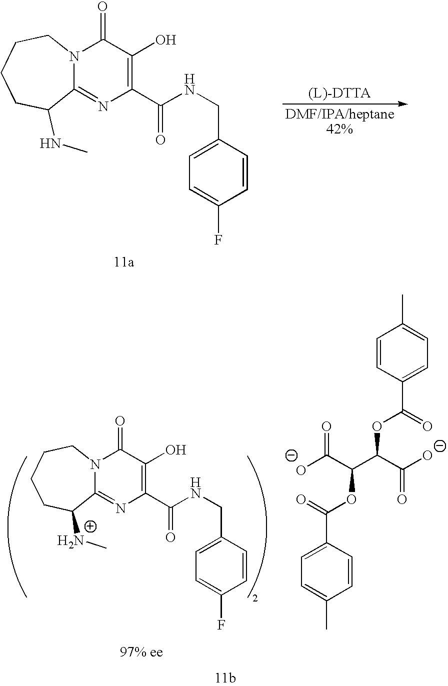

The racemic free amine 11a (97.9 wt %, 3.06 kg, 8.32 mol) was slurried in DMF (14 L) in a 100 L three-neck round bottom flask equipped with nitrogen inlet, reflux condenser, thermocouple and overhead mechanical stirring and heated to 50° C. Di-p-toluoyl-L-tartaric acid (98.9 wt %, 3.25 kg, 8.32 mol) was dissolved in DMF (7.0 L) and added to the amine slurry over 10 rnin with an addition fuimel. The reaction mixture was a slurry throughout the salt formation. The reaction mixture was seeded then cooled to 20° C. over 1 h. Isopropyl alcohol (14 L), then n-heptane (14 L) was added. The final solvent ratio is 3:2:2/DMF: Isopropanol: n-heptane. The slurry was aged at 20° C. for 2 h. The crystalline solid was filtered. The cake was washed with 2×7.5 L of 1:1/isopropanol: n-heptane, and dried at 40° C. under vacuum with a nitrogen sweep to afford chiral amine (L)-DTTA salt 11b (3.87 kg, 42% isolated yield, 97% ee). [α]_{D} −46.3° (c, 1.0, DMSO); ¹H NMR (400 MHz, CD₃OD) δ 7.96 (m, 2H), 7.37 (m, 2H), 7.23 (m, 2H), 7.02 (m, 2H), 5.87 (s, 1H), 5.09 (dd, J=14.4, 5.6 Hz, 1H), 4.55 (s, 2H), 4.48 (dd, J=10.8, 1.2 Hz, 1H), 3.46 (dd, J=14.4, 11.6 Hz, 1H), 2.76 (s, 3H), 2.26 (broad d, J=13.3 Hz, 1H), 2.08-1.84 (overlapped m, 3H), 1.69 (m, 1H), 1.37 (m, 1H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds, Flow Rate: 1 mL/min. Retention time for the amine: 8.118 min.; for (L)-DTTA: 12.637 min. Chiral HPLC: Column: Chiralpak AD, 250×4.6 mm; socratic 85:15Heptane: IPA with 0.2% TFA; Flow: 1.0 mL/min; Sample volume: 10 uL; Detector: UV @ 220 nm; Column Temperature: 30° C. Relative Retention Times: Undesired chiral amine: 0.79; (L)-DTTA: 0.91; Desired chiral amine: 1.00.

### Step 10: Preparation of Chiral Free Amine 11c

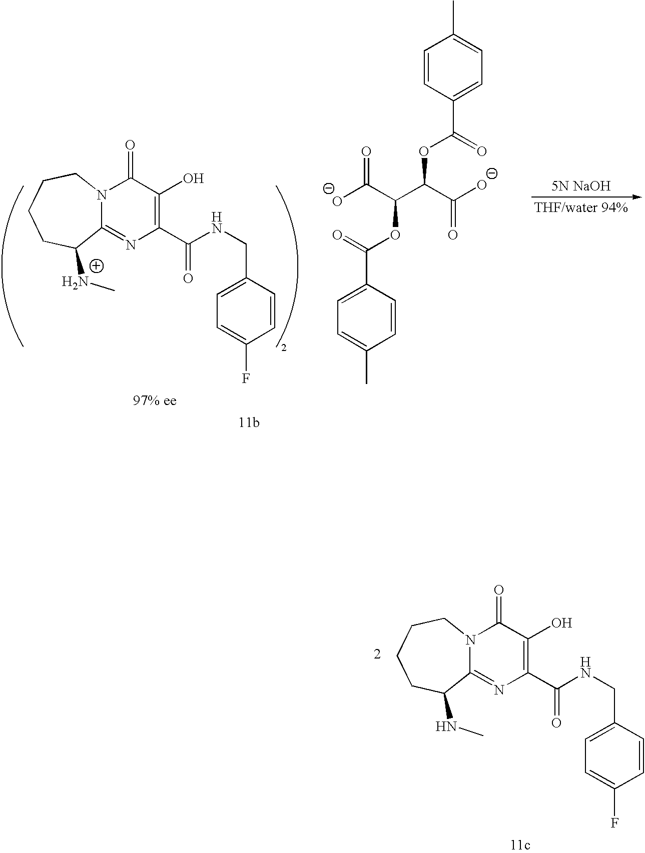

To a 100 L flask equipped with an overhead stirrer, thermocouple, nitrogen inlet and dropping funnel was charged THF (22 L) and GMP water (6.3 L). The di-p-toluoyl-L-tartrate salt 11b (4.2 kg, 65 wt % amine, 7.57 mol (amine), 1 eq.) was charged followed by THF rinse (3 L) to give a thick slurry. Aqueous sodium hydroxide (4.91 M, 1.54 L) was added all at once to the slurry. The addition of NaOH was exothermic and the thick slurry briefly became a thin slurry/solution prior to the crystallization of the free amine. After a 15 rnin age, GMP water (52.5 L) was added via the addition funnel. The water addition was exothermic and the batch temperature increased to ca. 28° C. The batch was aged for 2.5-3 h and cooled to 2-4° C. with ice-water to reduce the supernatant concentration to <2 mg/mL. The white solid was isolated by filtration and slurry washed twice with 8 L portions of GMP water. Two 8 L displacement washes with 1:1/MTBE:heptane were performed. The wet cake was dried in the filter pot under vacuum with nitrogen sweep to give chiral free amine 11c (2.57 kg, 94% yield after correction, 94 wt %, 97% ee). [α]_{D} −29.2° (c 1.1, DMSO).

### Step 11: Preparation of 14a

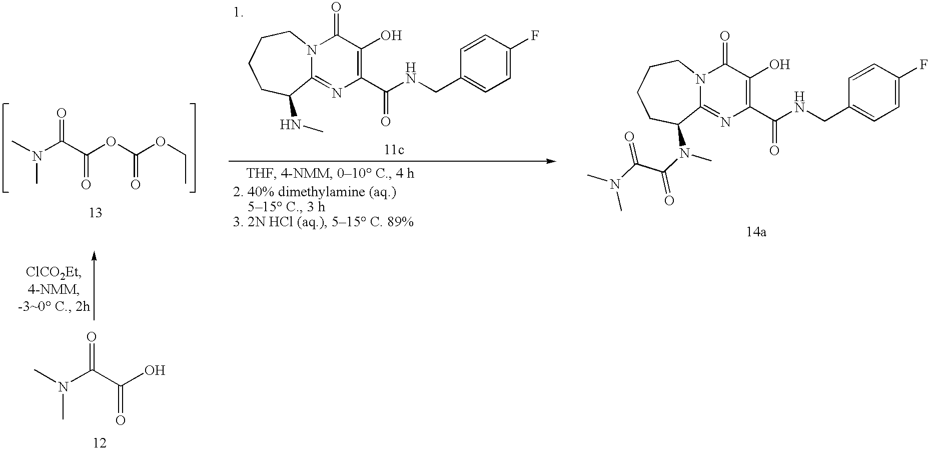

(1) Azeotropic drying of free amine: To a 100 L RBF equipped with an overhead stirrer, thermocouple, nitrogen inlet and batch concentrator was charged with THF (13 L) and free amine hydrate 11c (1.275 kg, 94 wt %). The slurry of free amine 11c was dried azeotropically with continuous distillation at about 60° C. under minimum vacuum with nitrogen sweep. Continuous distillation with about 15 vol of THF was typically resulted in KF=100 ppm. At this point, the total volume was about 12 L. The resulting solution was kept at room temperature under nitrogen.

(2) Mixed anhydride formation: To an another 50 L RBF, which was equipped with an overhead stirrer, thermocouple, nitrogen inlet and dropping funnel was charged with THF (18 L) and side chain acid 12 (0.663 kg). The resulting solution was cooled to 0° C. and ethyl chlorofonnate (0.478 L) was added. To the reaction mixture was dropwise added 4-NMM (0.586 L) at −3° C. to 0° C. over a period of 0.5 h, and aged for 2 h at the same temperature. The resulting slurry of mixed-anhydride 13 in THF (−5° C.) was transferred to the pre-cooled (−5˜8° C.) slurry of free amine IIc in THP. The reaction mixture was aged at 0˜5° C. for 1 h. At this point, an additional 4-NMM (0.550 L, 1.5 equiv) was charged and aged for 1.5 at 0-10° C. (typical conversion>95 A %, otherwise, more mixed-anhydride needed to be charged). Then, N,Ndimethylarnine aqueous solution (40% aq., 1.48 L) was added at 5-10° C., and aged for 2 h at 10-23° C. (holding point, or aged for 16 h). The reaction mixture was acidified by addition of 2 N HCl aqueous solution to adjust the pH to 3-4 at 5-15° C. The resulting reaction mixtures were transferred to 100 L extractor and added degassed brine (6 L). After a phase cut, the aqueous layer was back-extracted with 15 vol of EtOAc. The combined organic layer was further washed with brine (10 vol) and batch-concentrated at 20° C. at −23″ Hg (10 vol of additional EtOAc was used for the azeotrope). The final volume of EtOAc was adjusted to 12 L for the crystallization.

To the EtOAc solution was slowly added heptane (36 L) at room temperature. The resulting slurry was cooled to −3 to 2° C. over 0.5 h, and aged for 1 h. The crystalline solid was filtered, rinsed with cold (0° C.) EtOAc/heptane (1:3, 6 L), and dried under reduced pressure with nitrogen sweep for 5 h to give crude product 14a (1.40 kg, 92%).

(3) Recrystallization: The crude 14a (1.40 kg) and methanol (28 L) were charged in 50 L RBF, and heated to 45-50° C. Then, the resulting homogenous solution (35-40° C.) was transferred to another 72 L RBF via in-line filter. The methanol solution was cooled to 23° C. over 0.5 h and aged for 1 h at 23° C. The methanol slurry was batch-concentrated to a total volume (12 L). During distillation, the internal temp of the pot was at a range of 15-20° C. for the particle size. Then, degassed water (12 L) was added via in-line filter. A rapid addition of water was preferable at temperature ranges of 23-28° C. The resulting slurre was aged for 1 h at room temperature, then 2 h at −8˜5° C. The crystalline solid was filtered over filter pot, slurry-washed and rinsed with MeOH—H₂O (1:1.3, 3 L each). The wet cake was dried under vacuum with nitrogen sweep to give 14a as a non-hygroscopic crystalline solid (1.27 kg, 83% over yield, 99.8 A % purity, 99.8 wt % purity, >99.5% ee). [α]_{D} −86.3° (c 1.8, DMSO); ¹H NMR (CDCl₃, 400 MHz) δ:12.13 (s, 1H), 9.41 (br s, 1H), 7.38 (dd, J=8.5, 5.4 Hz, 2H), 7.00 (t, J=8.5 Hz, 2H), 5.40 (br s, 1H), 5.29 (dd, J=14.5, 6.0 Hz, 1H), 4.60 (dd, J=14.5, 6.6 Hz, 1H), 4.52 (dd, J=14.5, 6.3 Hz, 1H), 3.35 (dd, J=14.5, 11.6 Hz, 1H), 3.04 (s, 3H), 3.01 (s, 3H), 2.23-2.12 (m, 3H), 1.95-1.81 (m, 2H), 1.58-1.49 (m, 1H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time for 14a: 12.191 min.

### EXAMPLE 3

### Step 1: Preparation of O-Mesylated Bicyclic Pyrimidone 15

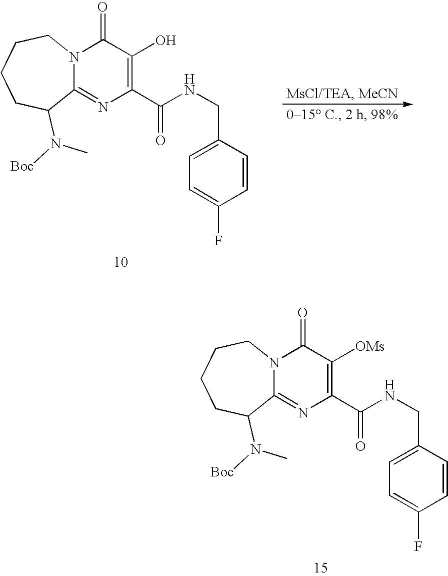

To a solution of bicyclic pyrimidone 10 (36.84) in acetonitrile (200 mL) was added TEA (12.3 mL) at rt. The resulting slurry was cooled to 0-5° C. To the slurry was slowly added methanesulfonyl chloride (6.5 mL) at 0-15° C. The resulting slurry was aged at 5-15° C. for 2 h (the reaction was monitored by HPLC). To the reaction mixture was slowly added water (450 mL). The resulting slurry was aged at 0° C. for 2 h. The crystalline solid was filtered off, washed with water (200 mL), haptane (100 mL), dried under vacuum with nitrogen sweep to afford desired O-Mesylated Bicyclic Pyrimidone 15 (42.09 g, 98%, >99 A % purity). ¹H NMR (CD₃CN, 400 MHz) δ:7.91 (br s, 0.3H, rotamer), 7.64 (br s, 0.7H, rotamer), 7.30 (br t, J=8.5 Hz, 2H), 7.04 (t, J=8.5 Hz, 2H), 5.40-5.15 (m, 1.7H), 5.03 (m, 0.3H), 4.65-4.46 (m, 2H), 3.55 (s, 3H), 3.50-3.33 (m, 1H), 2.84 (s, 3H), 2.23-2.05 (m, 3H), 1.85 (m, 1H), 1.73 (m, 1H), 1.43 (m, 1H), 1.30 (s, 9H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time for the O-Mesylated Bicyclic Pyrimidone 15: 14.769 min.

### Step 2: Preparation of O-Mesylated Bicyclic Pyrimidone Amine Hydrochloride Salt 16

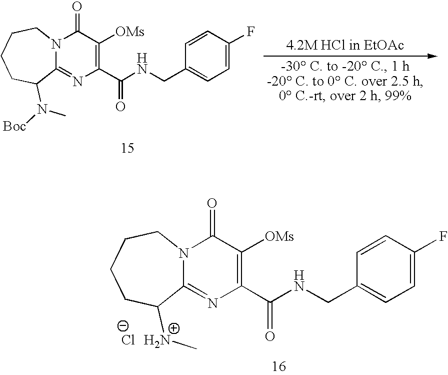

Vigorous stirring was requested for this step. To a 1 L round bottom flask was charged ethyl acetate (160 mL). To the solution of ethyl acetate was bubbled HCl gas (33.44 g, 10 eq.), at −30 to −20° C. O-Mesylated bicyclic pyrimidone 15 (crystalline solid, 49.34 g, 1 eq.) was slowly charged to the HCl-EtOAc solution at −30 to −20° C. The resulting solution was aged at −30 to −20° C. for 1 h, and slowly warmed to 0° C. over 2.5 h, then aged from 0° C. to rt over 2 h (100% conversion by HPLC). To the reaction mixture was diluted with EtOAc (188 mL), and slowly added heptane (376 mL) over 1 h. The resulting slurry was aged at rt for 1-2 h. The crystalline solid was filtered off, washed with heptane (100 mL), dried under vacuum with nitrogen sweep to afford desired product 16 (43.2 g, 99% isolated yield, >99 A % purity). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time for compound 16: 8.015 min.

### Step 3: Preparation of O-mesylated Free Amine 17

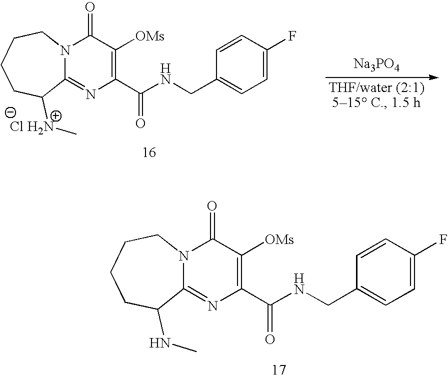

Vigorous stirring was required for this step. To a solution of amine-HCl salt 16 (37.74 g, 98.3% pure) in TBF/water (80 mL/40 mL) was slowly added Na₃PO₄ (14.09 g) in water (200 mL) at 5-15° C. The resulting slurry was aged at 5-15° C. for 0.5 h. To the slurry was added water (160 mL). The slurry was aged at 5° C. for 1 h. The crystalline solid was filtered off, washed with water (400 mL), heptane (100 mL) and dried under vacuum with nitrogen sweep to give desired free amine 17 (29.85 g, 87% yield, >99.5 A % purity). ¹H NMR (CD₃CN, 400 MHz) δ:8.41 (br s, 1H), 7.38 (dd, J=8.6, 2H), 7.09 (t, J=8.6 Hz, 2H), 4.92 (dd, J=14.2,4.8 Hz, 1H), 4.57-4.47 (m, 2H) Hz, 1H), 3.83 (d, J=9.5 Hz, 1H), 3.44 (s, 3H), 2.36 (s, 3H), 2.20-2.12 (m, 3H), 1.65-1.50 (m, 2H). HPLC conditions: Column: Zorbax, Rx C8 250×4.6 mm; Temperature: 30° C.; Detection at 210 nm; Mobile Phase: 0.1% aq H₃PO₄ (A)/MeCN (B); Gradient: 90:10 (A)/(B) to 10:90 over 15 min, 10:90 hold for 5 min, 10:90 to 90:10 (A)/(B) over 10 seconds; Flow Rate: 1 mL/min. Retention time for compound 17: 8.015 min.

### Step 4: Classical Resolution of O-Mesylated Free Amine 17

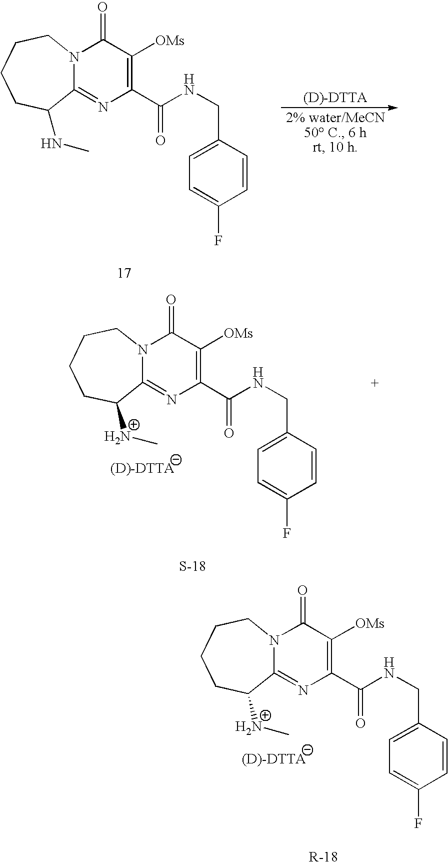

To a solution of (D)-DTTA (8.81 g) in 2% water/acetonitrile (80 mL) was slowly added free amine 17 (10.00 g) in-2% water/acetonitrile (40 mL) solution at 50° C. The resulting slurry was aged at 45-50° C. for 6 h, and at rt for 10 h. The crystalline solid was filtered off, washed with acetonitrile, dried under vacuum with nitrogen sweep to afford desired product S-18 (9.57 g, 90.1% ee, >99 A % purity, 51% yield). [α]_{D} −6.1 (c 1.7, DMSO); ¹H NMR (DMSO-d₆, 400 MHz) δ:9.14 (t, J=6.2 Hz, 1H), 7.81 (d, J=8.1 Hz,4H), 7.34 (dd, J=8.5, 5.8 Hz, 2H), 7.29 (d,J=8.1 Hz, 4H), 7.14 (dd, J=8.5, 5.8 Hz, 2H), 5.65 (s, 2H), 4.86 (dd, J=13.7, 5.4 Hz, 1H), 4.57 (br d, J=12.2 Hz, 1H), 4.44 (d, J=6.2 Hz, 2H), 3.69 (br t, J=12.2 Hz, 1H), 3.51 (s, 3H), 2.56 (s, 3H), 2.36 (s, 6H), 2.14 (m, 1H), 1.88 (m, 1H), 1.66 (m, 2H), 1.50 (m, 1H), 1.37 (m, 1H). Chiral SFC conditions: Column: OD-H; Temperature: 35° C.; Detection at 215 nm; Mobile Phase: 16% (25 mM i-BuNH₂ in MeOI/CO₂); Flow Rate: 1.5 mL/min; Pressure: 200 bar. Retention time for free amine S-17: 9.067 rnin.; for free amine R-17: 6.063 min; for (D)-DTTA: 3.284 min.

### Step 5: Neutralization of (R)-O-Mesylated Amine (D)-DTTA Salt R-18

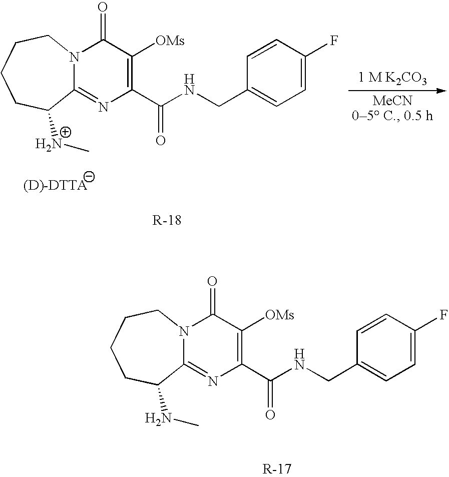

To a solution of amine-(D)-DTTA salt R-18 (˜0.01141 moles) in MeCN (35 mL) was slowly added 1 M of potassium carbonate (28.5 mL) at 0-5° C. The resulting solution was aged at 0-5° C. for 10 min. To the solution was added IPAc (50 mL), and stirred for 10 min. After a phase cut, the aqueous was back extracted with IPAc (30 mL). The combined organic layer was washed with brine (2×20 mL). The solution was concentrated and solvent-switched to acetonitrile (total volume 38 mL).

### Step 6: Racemization of O-Mesylated Free Amine R-17

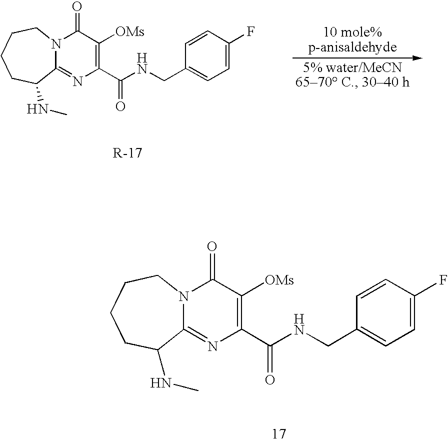

To a solution of free amine R-17 (0.01141 mol) in acetonitrile (38 mL) was added water (2 mL), and p-anisaldehyde (0.16 g). The resulting solution was degassed and heated at 65-70° C. for 30-40 h (0-3% ee monitored by chiral SFC). The resulting solution was used for classical resolution.

### Step 7: Classical Resolution of the First Recycle of O-Mesylated Free Amine 17

To a solution of the first recycle amine 17 from Step 6 above (about 0.01141 moles) in 5% water/acetonitrile was added 5.00 g of fresh free amine 17. The resulting solution was slowly added to a (D)-DTTA (8.81 g) in 2% water/acetonitrile (80 mL) at 50° C. The resulting slurry was aged at 45-50° C. for 6 h, and at rt for 10 h. The crystalline solid was filtered off, washed with acetonitrile, dried under vacuum with nitrogen sweep to afford desired product S-18 (8.82 g, 95.2% ee, 47% yield). The undesired product R-18 was taken through Steps 5-6 and the resulting second recycle amine 17 was used for classical resolution.

### Step 8: Classical Resolution of the Second Recycle of O-Mesylated Free Amine 17

To a solution of the second recycle amine 17 from Step 7 (about 0.01141 moles) in 5% water/acetonitrile was added 5.00 g of fresh free amine 17. The resulting solution was slowly added to a (D)-DTTA (8.81 g) in 2% water/acetonitrile (80 mL) at 50° C. The resulting slurry was aged at 45-50° C. for 6 h, and at rt for 10 h. The crystalline solid was filtered off, washed with acetonitrile, dried under vacuum with nitrogen sweep to afford desired product S-18 (7.94 g, 96.6% ee, 42% yield).

Thus, a total of 26.3 g (31.9 nmmol) of S-18 (average 93.8% ee, 70% overall yield) from 20.0 g (45.6 mmol) racemic amine 17 after two recycles.

### Step 9: Neutralization of Desired (S)—O-Mesylated Amine (D)-DTTA salt S-17

This process procedure is the same as above description. 27.20 g of combined (S)—O-mesylated amine (D)-DTTA salt S-18 gave 11.97 g of chiral free amine S-17 (83% yield, 94% ee, 98 A % purity). [α]_{D} −52.0° (c, 1.7, DMSO).

While the foregoing specification teaches the principles of the present invention, with an example provided for the purpose of illustration, the practice of the invention encompasses all of the usual variations, adaptations and/or modifications that come within the scope of the following claims.

1. A process for preparing a compound of Formula X or Formula XI: 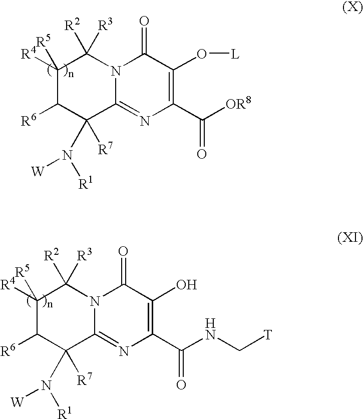
which comprises:
(H) contacting a compound of Formula VIII: 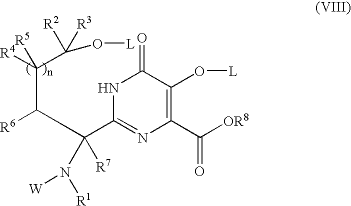
or a compound of Formula IX: 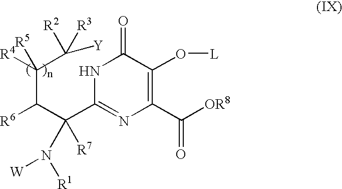
with a strong base to obtain Compound X; or
(H-1) contacting a compound of Formula VIII-1: 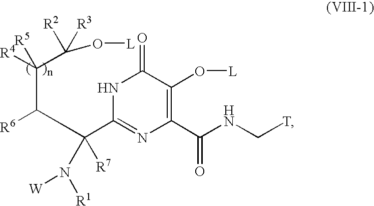
a compound of Formula VIII-2: 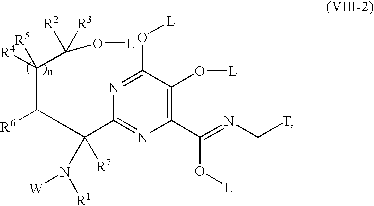
a compound of Formula VIII-3: 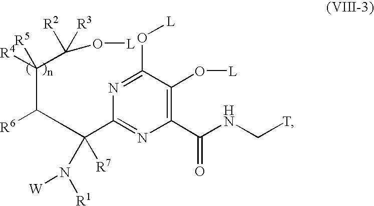
or a compound of Formula IX-1: 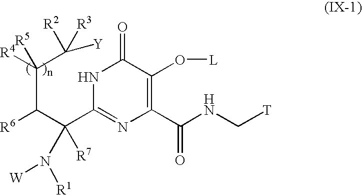
with a strong base to obtain Compound XI; wherein:
W is an amine protective group;
L is a hydroxy activating group;
Y is halo;
R¹ is:
(1) H,
(2) C₁₋₆ alkyl,
(3) C₁₋₆ alkyl substituted with O—C₁₋₆ alkyl, C₃₋₈ cycloalkyl, or aryl, wherein the cycloalkyl is optionally substituted with from 1 to 3 C₁₋₆ alkyl groups and the aryl is optionally substituted with from 1 to 5 substituents each of which is independently C₁₋₆ alkyl, O—C₁₋₆ alkyl, CF₃, OCF₃, halo, CN, or NO₂, or
(4) aryl which is optionally substituted with from 1 to 5 substituents each of which is independently C₁₋₆ alkyl, O—C₁₋₆ alkyl, CF₃, OCF₃, halo, CN, or NO₂;
R², R³, each R⁴, each R⁵, R⁶, and R⁷ are independently:
(1) H,
(2) C₁₋₆ alkyl, or
(3) C₁₋₆ alkyl substituted with O—C₁₋₆ alkyl, C₃₋₈ cycloalkyl, or aryl, wherein the cycloalkyl is optionally substituted with from 1 to 3 C₁₋₆ alkyl groups and the aryl is optionally substituted with from 1 to 5 substituents each of which is independently C₁₋₆ alkyl, O—C₁₋₆ alkyl, CF₃, OCF₃, halo, CN, or NO₂;
R⁸ is (i) a mixture of R^{A} and R^{B}, wherein R^{A} and R^{B} are different C₁₋₆ alkyl groups, or is (ii) R^{C}, wherein R^{C} is a C₁₋₆ alkyl;
each aryl is independently phenyl or naphthyl;
n is an integer equal to zero, 1, 2 or 3;
T is 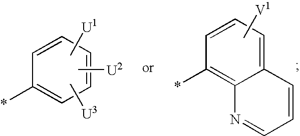
U¹, U² and U³ are each independently selected from the group consisting of H, halo, C₁₋₆ alkyl, O—C1-6 alkyl, C₁₋₆ fluoroalkyl, SO₂-C₁₋₆ alkyl, C(═O)—NH(—C₁₋₆ alkyl), C(═O)—N(—C₁₋₆ alkyl)₂, and HetA;
V¹ is H, halo, C₁₋₆ alkyl, or C₁₋₆ fluoroalkyl; and
each HetA is independently a 5- or 6-membered heteroaromatic ring containing from 1 to 4 heteroatoms independently selected from N, O and S, wherein the heteroaromatic ring is optionally substituted with 1 or 2 C₁₋₆ alkyl groups.

2. The process according to claim 1, wherein L is:
(1) SO₂R^{I},
(2) P(O)(R^{J})₂, or
(3) P(O)(—OR^{K})₂;
wherein
R^{I} is (i) C₁₋₆ alkyl, (ii) C₁₋₆ haloalkyl, (iii) C₁₋₆ alkyl substituted with aryl, (iv) aryl, or (v) camphoryl;
each R^{J} is independently (i) C₁₋₆ alkyl, (ii) C₁₋₆ haloalkyl, (iii) C₁₋₆ alkyl substituted with aryl, or (iv) aryl; and
each R^{K} is independently (i) C₁₋₆ alkyl or (ii) C₁₋₆ alkyl substituted with aryl; and
wherein any aryl defined in R^{I}, R^{J}, and R^{K} is optionally substituted with from 1 to 5 substituents each of which is independently halogen, —C₁₋₄ alkyl, —O—C₁₋₄ alkyl, CF₃, OCF₃, CN, or nitro.

3. The process according to claim 1, wherein W is an amine protective group selected from the group consisting of:
(1) C₁₋₆ alkyl substituted with aryl, where the aryl is optionally substituted with from 1 to 5 substituents each of which is independently halo, NO₂, —C₁₋₄ alkyl, or —O—C₁₋₄ alkyl,
(2) C(═O)—C₁₋₄ alkyl,
(3) C(═O)—C₁₋₄ haloalkyl,
(4) C(═O)—C₁₋₄ alkylene-aryl, where the aryl is optionally substituted with from 1 to 5 substituents each of which is independently halo, —NO₂, —C₁₋₄ alkyl, or —O—C₁₋₄ alkyl,
(5) C(═O)—O—C₁₋₄ alkyl,
(6) C(═O)—O—(CH₂)₀₋₁—CH═CH₂, and
(7) C(═O)—O—C₁₋₄ alkylene-aryl, where the aryl is optionally substituted with from 1 to 5 substituents each of which is independently halo, —NO₂, —C₁₋₄ alkyl, or —O—C₁₋₄ alkyl.

4. The process according to claim 1, wherein R², R³, each R⁴, each R⁵, R⁶, and R⁷ are all H.

5. The process according to claim 1, wherein the strong base in Step H or Step H-1 is selected from the group consisting of the alkali metals, alkali metal and alkaline earth metal halides, Group 2b transition metal halides, alkali metal salts and alkaline earth metal salts of di-C₁-C₆ alkylamines and C₄-C₈ cyclic secondary amines, alkali metal salts and alkaline earth metal salts of bis(tri-C₁₋₄ alkylsilyl)amines, alkali metal and alkaline earth metal hydrides, C₁₋₆ alkyllithiums, aryllithiums, mono- and di-(C₁₋₆ alkyl)aryllithiums, C₁₋₆ alkylmagnesium halides, arylmagnesium halides, alkali metal amides, C₁₋₆ alkoxides of alkali and alkaline earth metals, alkali metal carbonates and bicarbonates, alkali metal phosphates, and alkali metal and alkaline earth metal hydroxides.

6. The process according to claim 1, which further comprises:
(F1) treating a compound of Formula VII: 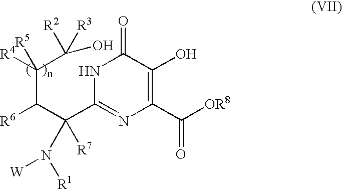
with a hydroxy activating agent to form a product which is (i) the compound of Formula VIII, (ii) a compound of Formula VIIIa: 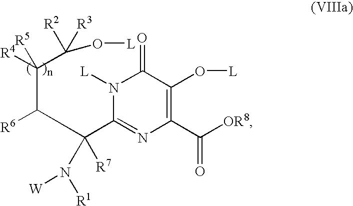
or (iii) a mixture of Compound VIII and Compound VIIIa;
(F2) then:
(1) when the product is (i) Compound VIII, proceeding directly to Step G or to Step H;
(2) when the product is (ii) Compound VIIIa, contacting the product with (a) a primary or secondary amine or (b) an alcohol, water, or an alcohol-water mixture in the presence of a base, to form Compound VIII; and
(3) when the product is (iii) a mixture of Compounds VIII and VIIIa, optionally contacting the product with (a) a primary or secondary amine or (b) an alcohol, water, or an alcohol-water mixture in the presence of a base, to form additional Compound VIII; and
(G) optionally reacting Compound VIII from Step F2 with a halide salt to form the compound of Formula IX; or
(F1-1) reacting a compound of Formula VIII with an amine of formula T-CH₂NH₂ to obtain a compound of Formula VIII-1: 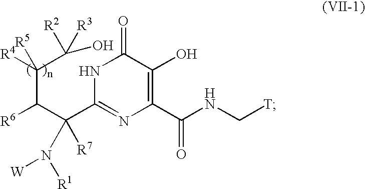
(F1-2) treating a compound of Formula VIII-1 with a hydroxy activating agent to form a product which is (i) a compound of Formula VIII-1, (ii) a compound of Formula VIII-2, (iii) a compound of Formula VIII-3, (iv) a compound of Formula VIII-1a, or (v) a mixture of two to four components selected from the group consisting of Compound VIII-1, Compound VIII-2, Compound VIII-3 and Compound VIII-1a; 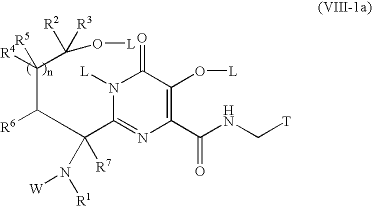
(F2-1) then:
(1) when the product is (i) a compound of Formula VIII-1, (ii) a compound of Formula VIII-2, (iii) a compound of Formula VIII-3, or a mixture thereof, proceeding directly to Step G-1 or to Step H-1;
(2) when the product is (iv) Compound VIII-1a, contacting the product with (a) a primary or secondary amine or (b) an alcohol, water, or an alcohol-water mixture in the presence of a base, to form Compound VIII-1; and
(3) when the product is the mixture (v) containing VIII-1a, optionally contacting the product with (a) a primary or secondary amine or (b) an alcohol, water, or an alcohol-water mixture in the presence of a base, to form additional Compound VIII-1; and
(G-1) optionally reacting Compound VIII-1 from Step F2-1 with a halide salt to form a compound of Formula IX-1.

7. The process according to claim 6, wherein the activating agent in Step F1 or Step F1-2 is an agent of formula L-X; wherein L is R^{I}SO₂, (R^{J})₂P(O), or (R^{K}O)₂P(O) and X is halogen; wherein R^{I} is (i) C₁₋₆ alkyl, (ii) C₁₋₆ haloalkyl, (iii) C₁₋₆ alkyl substituted with aryl, (iv) aryl, or (v) camphoryl; each R^{J} is independently (i) C₁₋₆ alkyl, (ii) C₁₋₆ haloalkyl, (iii) C₁₋₆ alkyl substituted with aryl, or (iv) aryl; and each R^{K} is independently (i) C₁₋₆ alkyl or (ii) C₁₋₆ alkyl substituted with aryl; and wherein any aryl defined in R^{I}, R^{J}, and R^{K} is optionally substituted with from 1 to 5 substituents each of which is independently halogen, —C₁₋₄ alkyl, —O—C₁₋₄ alkyl, CF₃, OCF₃, CN, or nitro.

8. A process according to claim 1 which further comprises Steps I, J, and optionally J^{a}:
(I) reacting an amine of formula T-CH₂N₂ with the compound of Formula X obtained from Step H to obtain a compound of Formula XI; and then
(J) treating the compound of Formula XI obtained from Step I or from Step H-1 with an amine deprotecting agent to remove group W and obtain a compound of Formula XII: 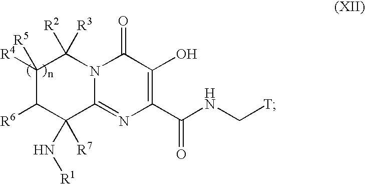
and then, when the compound of Formula XII is racemic, optionally:
(J^{a}) converting the compound of Formula XII to an enantiomerically-enriched form wherein the amount of (S)-Compound XII is greater than the amount of (R)-Compound XII.
or which further comprises Steps I and I^{a}:
(I) reacting an amine of formula T-CH₂NH₂ with the compound of Formula X obtained from Step H to obtain a compound of Formula XI; and then
(I^{a}) (i) reacting the compound of Formula XI obtained from Step I or Step H-1with a hydroxy activating agent to form a racemic compound of Formula XIa: 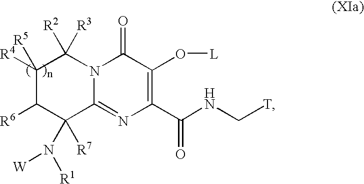
(ii) treating the compound of Formula XI with an amine deprotecting agent to remove group W and obtain a compound of Formula XIIa: 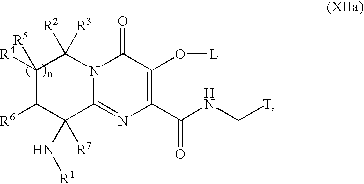
(iii) converting the racemic compound of Formula XIIa to an enantiomerically-enriched form wherein the amount of (S)-Compound XIIa is greater than the amount of (R)-Compound XIIa, and
(iv) removing the L group from the enantiomerically-enriched form of Compound XIIa to obtain an enantiomerically enriched form of a compound of Formula XII.

9. A process according to claim 8 which further comprises:
(L) either (i) reacting the compound of Formula XII obtained from Step J with (i) (R^{M}R^{N})N—C(═O)—C(═O)—OC(═O)—O—C₁₋₆ alkyl, or (ii) reacting the compound of Formula XII with R^{F}O—C(═O)—C(═O)-Z and then with (R^{M}R^{N})NH, to obtain a compound of Formula XIV: 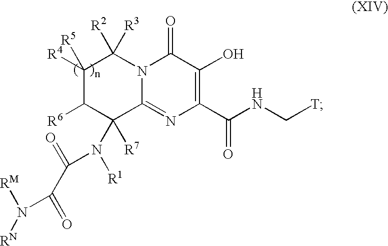
or
(La) either (i) reacting the enatiomerically enriched form of the compound of Formula XII obtained from Step I^{a} or J^{a}with (i) (R^{M}R^{N})N—C(═O)—C(═O)—OC(═O)—OC₁₋₆ alkyl, or (ii) reacting the compound of Formula XII with R^{F}O—C(═O)—C(═O)-Z and then with (R^{M}R^{N})NH, to obtain an enantiomerically enriched form of Compound XIV;
wherein R^{M} and R^{N} are each independently C₁₋₆ alkyl or C₁₋₆ alkyl substituted with aryl, or alternatively R^{M} and R^{N} together with the N to which both are attached form C₄₋₇ azacycloalkyl;
R^{F} is C₁₋₆ alkyl; and
Z is halo or OH.

10. A process for preparing a compound of Formula XX or Formula XI: 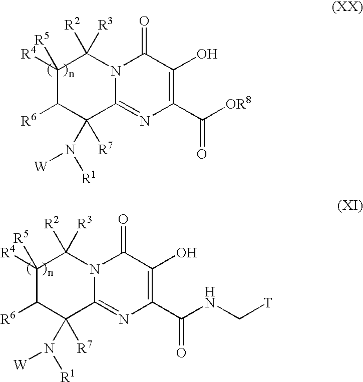
which comprises:
(HZ) treating a compound of Formula VII or Formula VII-1: 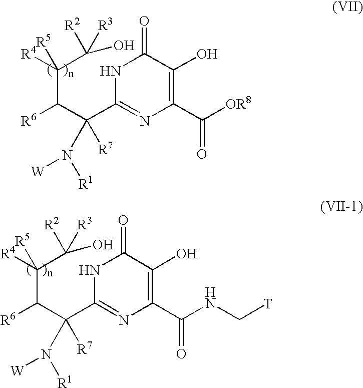
with a trihydrocarbylphosphine reagent in the presence of an azodicarboxylate of Formula R^{Y}O₂C—N═N—CO₂R^{Z} to form the compound of Formula XX or XI, respectively; wherein:
W is an amine protective group;
R¹ is:
(1) H,
(2) C₁₋₆ alkyl,
(3) C₁₋₆ alkyl substituted with O—C₁₋₆ alkyl, C₃₋₈ cycloalkyl, or aryl, wherein the cycloalkyl is optionally substituted with from 1 to 3 C₁₋₆ alkyl groups and the aryl is optionally substituted with from 1 to 5 substituents each of which is independently C₁₋₆ alkyl, O—C₁₋₆ alkyl, CF₃, OCF₃, halo, CN, or NO₂, or
(4) aryl which is optionally substituted with from 1 to 5 substituents each of which is independently C₁₋₆ alkyl, O—C₁₋₆ alkyl, CF₃, OCF₃, halo, CN, or NO₂; R², R³, each R⁴, each R⁵, R⁶, and R⁷ are independently:
(1) H,
(2) C₁₋₆ alkyl, or
(3) C₁₋₆ alkyl substituted with O—C₁₋₆ alkyl, C₃₋₈ cycloalkyl, or aryl, wherein the cycloalkyl is optionally substituted with from 1 to 3 C₁₋₆ alkyl groups 1 and the aryl is optionally substituted with from 1 to 5 substituents each of which is independently C₁₋₆ alkyl, O—C₁₋₆ alkyl, CF₃, OCF₃, halo, CN, or NO₂;
R⁸ is (i) a mixture of R^{A} and R^{B}, wherein R^{A} and R^{B} are different C₁₋₆ alkyl groups, or is (ii) R^{C}, wherein R^{C} is a C₁₋₆ alkyl;
R^{Y} and R^{Z} are each independently C₁₋₆ alkyl;
each aryl is independently phenyl or naphthyl;
n is an integer equal to zero, 1, 2 or 3;
T is 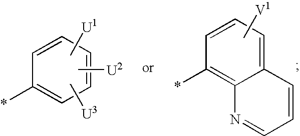
U¹, U² and U³ are each independently selected from the group consisting of H, halo, C₁₋₆ alkyl, O—C₁₋₆ alkyl, C₁₋₆ fluoroalkyl, SO₂-C₁₋₆ alkyl, C(═O)—NH(—C₁₋₆ alkyl), C(═O)—N(—C₁₋₆ alkyl)₂, and HetA;
V¹ is H, halo, C₁₋₆ alkyl, or C₁₋₆ fluoroalkyl; and each HetA is independently a 5- or 6-membered heteroaromatic ring containing from 1 to 4 heteroatoms independently selected from N, O and S, wherein the heteroaromatic ring is optionally substituted with 1 or 2 C₁₋₆ alkyl groups.

11. A compound of Formula VIIIb or VIIb-1: 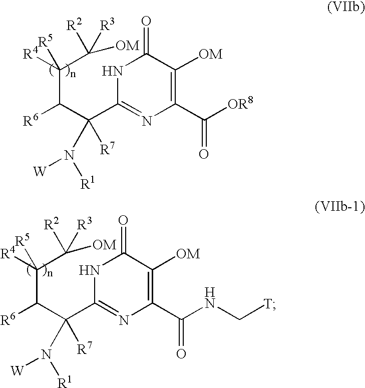
wherein:
each M is H or a hydroxy activating group;
W is an arnine protective group;
R¹ is:
(1) H,
(2) C₁₋₆ alkyl,
(3) C₁₋₆ alkyl substituted with O—C₁₋₆ alkyl, C₃₋₈ cycloalkyl, or aryl, wherein the cycloalkyl is optionally substituted with from 1 to 3 C₁₋₆ alkyl groups and the aryl is optionally substituted with from 1 to 5 substituents each of which is independently C₁₋₆ alkyl, O—C₁₋₆ alkyl, CF₃, OCF₃, halo, CN, or NO₂, or
(4) aryl which is optionally substituted with from 1 to 5 substituents each of which is independently C₁₋₆ alkyl, O—C₁₋₆ alkyl, CF₃, OCF₃, halo, CN, or NO₂; R², R³, each R⁴, each R⁵, R⁶, and R⁷ are independently:
(1) H,
(2) C₁₋₆ alkyl, or
(3) C₁₋₆ alkyl substituted with O—C₁₋₆ alkyl, C₃₋₈ cycloalkyl, or aryl, wherein the cycloalkyl is optionally substituted with from 1 to 3 C₁₋₆ alkyl groups and the aryl is optionally substituted with from 1 to 5 substituents each of which is independently C₁₋₆ alkyl, O—C₁₋₆ alkyl, CF₃, OCF₃, halo, CN, or NO₂;
R⁸ is (i) a mixture of R^{A}and R^{B}, wherein R^{A} and R^{B} are different C₁₋₆ alkyl groups, or is (ii) R^{C}, wherein R^{C} is a C₁₋₆ alkyl;
each aryl is independently phenyl or naphthyl;
n is an integer equal to zero, 1, 2 or 3;
T is 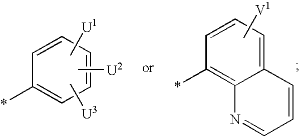
U¹, U² and U³ are each independently selected from the group consisting of H, halo, C₁₋₆ alkyl, O—C₁₋₆ alkyl, C₁₋₆ fluoroalkyl, SO₂-C₁₋₆ alkyl, C(═O)—NH(—C₁₋₆ alkyl), C(═O)—N(—C₁₋₆ alkyl)₂, and HetA;
V^{I} is H, halo, C₁₋₆ alkyl, or C₁₋₆ fluoroalkyl; and
each HetA is independently a 5- or 6-membered heteroaromatic ring containing from 1 to 4 heteroatoms independently selected from N, O and S, wherein the heteroaromatic ring is optionally substituted with 1 or 2 C₁₋₆ alkyl groups.

12. A compound selected from:

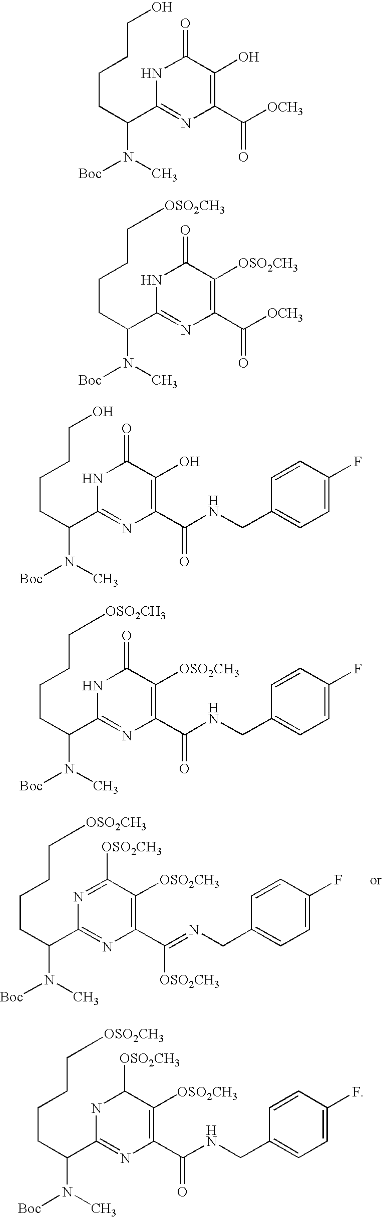

13. A compound of Formula VId: 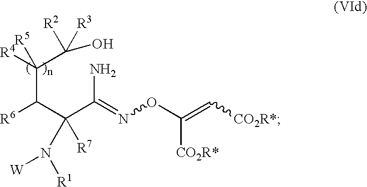
wherein W is an amine protective group;
each R* is independently a C₁₋₆ alkyl group;
R¹ is:
(1) H,
(2) C₁₋₆ alkyl,
(3) C₁₋₆ alkyl substituted with O—C₁₋₆ alkyl, C₃₋₈ cycloalkyl, or aryl, wherein the cycloalkyl is optionally substituted with from 1 to 3 C₁₋₆ alkyl groups and the aryl is optionally substituted with from 1 to 5 substituents each of which is independently C₁₋₆ alkyl, O—C₁₋₆ alkyl, CF₃, OCF₃, halo, CN, or NO₂, or
(4) aryl which is optionally substituted with from 1 to 5 substituents each of which is independently C₁₋₆ alkyl, O-C₁₋₆ alkyl, CF₃, OCF₃, halo, CN, or NO₂; R², R³, each R⁴, each R⁵, R⁶, and R⁷ are independently:
(1) H,
(2) C₁₋₆ alkyl, or
(3) C₁₋₆ alkyl substituted with O-C₁₋₆ alkyl, C₃₋₈ cycloalkyl, or aryl, wherein the cycloalkyl is optionally substituted with from 1 to 3 C₁₋₆ alkyl groups and the aryl is optionally substituted with from 1 to 5 substituents each of which is independently C₁₋₆ alkyl, O—C₁₋₆ alkyl, CF₃, OCF₃, halo, CN, or NO₂;
each aryl is independently phenyl or naphthyl; and
n is an integer equal to zero, 1, 2 or 3.

14. A compound of Formula V: 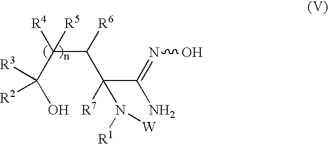
wherein W is an amine protective group;
R¹ is:
(1) H,
(2) C₁₋₆ alkyl,
(3) C₁₋₆ alkyl substituted with O—C₁₋₆ alkyl, C₃₋₈ cycloalkyl, or aryl, wherein the cycloalkyl is optionally substituted with from 1 to 3 C₁₋₆ alkyl groups and the aryl is optionally substituted with from 1 to 5 substituents each of which is independently C₁₋₆ alkyl, O—C₁₋₆ alkyl, CF₃, OCF₃, halo, CN, or NO₂, or
(4) aryl which is optionally substituted with from 1 to 5 substituents each of which is independently C₁₋₆ alkyl, O—C₁₋₆ alkyl, CF₃, OCF₃, halo, CN, or NO₂;
R², R³, each R⁴, each R⁵, R⁶, and R⁷ are independently:
(1) H,
(2) C₁₋₆ alkyl, or
(3) C₁₋₆ alkyl substituted with O—C₁₋₆ alkyl, C₃₋₈ cycloalkyl, or aryl, wherein the cycloalkyl is optionally substituted with from 1 to 3 C₁₋₆ alkyl groups and the aryl is optionally substituted with from 1 to 5 substituents each of which is independently C₁₋₆ alkyl, O—C₁₋₆ alkyl, CF₃, OCF₃, halo, CN, or NO₂;
each aryl is independently phenyl or naphthyl; and
n is an integer equal to zero, 1, 2 or 3.

15. A compound which is a compound of Formula III or a compound of Formula IV: 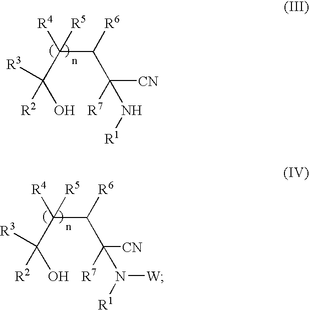
wherein W is an amine protective group;
R¹ is:
(1) H,
(2) C₁₋₆ alkyl,
(3) C₁₋₆ alkyl substituted with O—C₁₋₆ alkyl, C₃₋₈ cycloalkyl, or aryl, wherein the cycloalkyl is optionally substituted with from 1 to 3 C₁₋₆ alkyl groups and the aryl is optionally substituted with from 1 to 5 substituents each of which is independently C₁₋₆ alkyl, O—C₁₋₆ alkyl, CF₃, OCF₃, halo, CN, or NO₂, or
(4) aryl which is optionally substituted with from 1 to 5 substituents each of which is independently C₁₋₆ alkyl, O—C₁₋₆ alkyl, CF₃, OCF₃, halo, CN, or NO₂;
R², R³, each R⁴, each R⁵, R⁶, and R⁷ are independently:
(1) H,
(2) C₁₋₆ alkyl, or
(3) C₁₋₆ alkyl substituted with O—C₁₋₆ alkyl, C₃₋₈ cycloalkyl, or aryl, wherein the cycloalkyl is optionally substituted with from 1 to 3 C₁₋₆ alkyl groups and the aryl is optionally substituted with from 1 to 5 substituents each of which is independently C₁₋₆ alkyl, O—C₁₋₆ alkyl, CF₃, OCF₃, halo, CN, or NO₂;
each aryl is independently phenyl or naphthyl; and
n is an integer equal to zero, 1, 2 or 3.
